(19) United States
(12) Patent Application Publication
(10) Pub. No.: US 2024/0398876 A1
(43) Pub. Date: Dec. 5, 2024

(54) BIOLOGICAL MARKER OF INTESTINAL DYSBIOSIS, USEFUL FOR PREDICTING THE RESPONSE OF A CANCER PATIENT TO AN ANTI-PDI DRUG

(71) Applicants: INSTITUT GUSTAVE ROUSSY, VILLEJUIF (FR); EVERIMMUNE, VILLEJUIF (FR)

(72) Inventors: Lisa DEROSA, PARIS (FR); Romain DAILLERE, ANGERS (FR); Laurence ZITVOGEL, PARIS (FR)

(21) Appl. No.: 18/261,598

(22) PCT Filed: Jan. 19, 2022
(86) PCT No.: PCT/EP2022/051155

§ 371 Date: Jul. 14, 2023

(30) Foreign Application Priority Data
Jan. 19, 2021 (EP) 21305064.4
Jan. 29, 2021 (EP) 21305130.3

(51) Int. Cl. A61K 35/741; A61K 35/00; A61K 35/24; C12Q 1/689

(52) U.S. Cl. CPC A61K 35/741; A61K 35/24; C12Q 1/689; A61K 2035/115; C12Q 2600/106; C12Q 2600/158

(57) ABSTRACT

The present invention relates to the field of anticancer treatment. In particular, the present invention concerns the role of the gut microbiota in the efficacy of immune checkpoints inhibitors (ICI)-based treatments and provides methods for determining if a patient is likely to benefit from an ICI-based treatment, more precisely, treatment comprising administration of an antibody directed against PD1 or PD-L1. The present invention provides a method for stratifying cancer patients according to their potential need of bacterial complementation before receiving an ICI-based treatment. The present invention also provides a simple method for determining whether an individual has an intestinal dysbiosis.

### FIELD OF THE INVENTION

The present invention relates to the field of anticancer treatment. In particular, the present invention concerns the role of the gut microbiota in the efficacy of immune checkpoints inhibitors (ICI)-based treatments and provides methods for determining if a patient is likely to benefit from an ICI-based treatment, more precisely, treatment comprising administration of an antibody directed against PD1 or PD-L1.

The present invention provides a method for stratifying cancer patients according to their potential need of bacterial complementation before receiving an ICI-based treatment.

The present invention also provides a simple method for determining whether an individual has an intestinal dysbiosis.

### BACKGROUND OF THE INVENTION

The development of immune checkpoint inhibitors (ICI) targeting the PD-1/PD-L1 interaction has transformed the therapeutic landscape of patients with advanced non-small cell lung cancer (NSCLC)(Herbst et al. 2016; Brahmer et al. 2015; Borghaei et al. 2015; Gandhi et al. 2018; Paz-Ares et al. 2018; Reck et al. 2016). Landmark trials performed on previously treated patients with advanced NSCLC demonstrated superior overall survival (OS) with PD-1/PD-L1 blockade compared to standard chemotherapy (Herbst et al. 2016; Brahmer et al. 2015; Borghaei et al. 2015). Following unprecedented OS results from phase Ill randomized trials on patients with previously untreated advanced NSCLC, ICI were approved in the first-line setting, either as monotherapy for patients with tumor PD-L1 expression 50% on tumor cells or in combination with platinum-doublet chemotherapy irrespectively of PD-L1 expression (Gandhi et al. 2018; Paz-Ares et al. 2018; Reck et al. 2016; Arielle Elkrief et al. 2020). However, only a minority (35%) of patients benefit from sustained response to ICI (Gadgeel et al. 2020). The majority of NSCLC patients develop primary or secondary resistance, or occasional acceleration of the disease called “hyper-progression” (Ferrara et al. 2018). Furthermore, current biomarkers of response to ICI are not satisfactory due to low sensitivity and specificity, and therefore, understanding mechanisms of resistance to ICI to identify robust biomarkers of resistance are urgently needed.

Primary resistance has been attributed to low tumor mutational burden and poor intrinsic antigenicity of tumor cells (Riaz et al. 2017; Rizvi et al. 2015), defective antigen presentation (Spranger, Bao, et Gajewski 2015), limited intratumoral infiltration related to T cell exhaustion (Smyth et al. 2016), and metabolic immunosuppressive pathways (Koyama et al. 2016; Young et al. 2016). High dimensional omics technologies are currently developed to decipher the main regulators of the “cancer immune set-point”—the threshold beyond which an effective immune response can occur in the tumor bearer (Chen et Mellman 2017).

Recent lines of evidence point to the biological significance of the composition of the gut microbiota in influencing peripheral immune tonus and effectiveness of ICI in patients with cancer. The human gut microbiome, composed of 10¹³ micro-organisms, modulates many host processes including metabolism, inflammation, peristalsis, elimination of pathogens and xenobiotics, maturation of immune functions to maintain tolerance to microbial and food antigens as well as the intestinal epithelial barrier fitness (Routy, Gopalakrishnan, et al. 2018). More recently, the gut microbiome has unexpectedly been shown to influence the effectiveness of ICI (Routy, Le Chatelier, et al. 2018). First, in pre-clinical models, experiments with germ-free or antibiotic (ATB)-treated mice unraveled that the antitumor activity of ICI requires the presence of gut microbial components (Vetizou et al. 2015). Similarly, ATB prior to ICI initiation drastically reduced the clinical benefit and survival in several cohorts of patients across various cancer types (melanoma, advanced NSCLC, renal cell carcinoma (RCC), and urothelial cancer)(Routy, Le Chatelier, et al. 2018; Gopalakrishnan et al. 2018; L. Derosa et al. 2018; Lisa Derosa et Zitvogel 2020). This observation was confirmed in prospective trials and large meta-analyses, suggesting that gut microbiota may be instrumental for the immunostimulatory mode of action of ICI (Lurienne et al. 2020).

Supporting this contention, we and others reported that primary responses to anti-PD-1/PD-L1 antibodies in patients with epithelial tumours and melanoma could, at least in part, be attributed to the composition of the gut microbiota (Routy, Le Chatelier, et al. 2018; Gopalakrishnan et al. 2018; Matson et al. 2018). A diverse microbiota and the presence of specific bacteria such as Akkermansia muciniphila, Ruminococcus or Bifidobacterium genera were associated with improved clinical response to ICI, correlating with increased systemic immune tonus (Routy, Le Chatelier, et al. 2018; Gopalakrishnan et al. 2018; Matson et al. 2018). Our previous work reported the metagenomics-based microbiome profiling of 100 patients diagnosed with refractory NSCLC or RCC treated with second or third line anti-PD-1 antibodies, concluding that the prevalence of A. muciniphila (Akk) was increased in patients presenting partial responses or stable disease, as compared with patients in progressive disease (61 versus 34% respectively, p=0.007)(Routy, Le Chatelier, et al. 2018). Akk was also overrepresented in patients with progression free-survival (PFS)>3 months, when analyzing a subgroup of 60 patients with NSCLC (Routy, Le Chatelier, et al. 2018). Another group performed 16S rRNA gene amplicon sequencing of 37 NSCLC patient feces, confirming that Akk was enriched in patients responding to ICI (Jin et al. 2019). In patients with prostate adenocarcinomas treated with abiraterone, intestinal Akk also correlated with therapeutic responses (Daisley et al. 2020). Akk has been associated in clinical specimens with low body mass index, fitness, general health and successful aging (as indicated by its presence in disease-free centenarians)(Santoro et al. 2018). In animal models, Akk supplementation reduces obesity (Zhou et al. 2020) and its co-morbidities, palliates neurodegenerative disorders (Blacher et al. 2019) and counteracts progeria (Bárcena et al. 2019). Therefore, Akk may be viewed as a potential master regulator of homeostasis in the metaorganism.

Altogether, these preliminary results in small cohorts from a heterogeneous geographical distribution raise the question as to whether the gut microbiome composition, and more specifically the presence of Akk at diagnosis, could represent a potential biomarker that predicts the response of patients with advanced NSCLC and other cancers to ICI. In order to validate this hypothesis, we performed a prospective multicentric study and analyzed the metagenomics-based microbiome profile of 311 patients with advanced NSCLC treated with anti-PD-1 antibodies.

### SUMMARY OF THE INVENTION

The inventors previously showed that the absence of Akkermansia muciniphila in a feces sample from a cancer patient is indicative of a resistance to PD1 blockade (WO2018115519).

In the experiments reported below, the inventors surprisingly identified a group of patients exhibiting high levels Akkermansia muciniphila which correlated with a poor outcome.

They thus considered a trichotomic stratification of patients into Akk⁻, Akk^{low} (<75^{th} percentile) and Akk^{high} (>75^{th} percentile) individuals, which generated a more accurate independent predictor of overall survival than the dichotomic (Akk⁻ versus Akk⁺) division. Using this 3-groups patient stratification, Akkermansia muciniphila level represented a more reliable biomarker of prognosis for patients receiving immunotherapy with PD-1 blockade, even overruling PD-L1 as a predictive biomarker of response to ICI in ≥2L NSCLC patients.

They then demonstrated that Akkermansia SGB9228 behave the same way as Akkermansia muciniphila, i.e., the presence of “normal levels” of A. muciniphila (A. muciniphila^{low}) or Akkermansia SGB9228 (A. SGB9228^{low}) in the gut, can be considered as a surrogate of host intestinal fitness. Conversely, in the absence of bacteria of the Akkermansia genus in the host gut microbiota, or in their presence at excessively high levels (especially excessively high levels of A. muciniphila and/or Akkermansia SGB9228), the host needs a bacterial compensation for responding to an ICI-therapy.

Hence, according to a first aspect, the present invention pertains to an in vitro theranostic method to determine if a cancer patient is likely to be a good responder to an immune checkpoint inhibitor (ICI)-based therapy, comprising measuring, in a sample from said patient, the relative abundance of bacteria of the Akkermansia genus, for example Akkermansia muciniphila and/or Akkermansia SGB9228, wherein the presence of said Akkermansia below a predetermined threshold is indicative that the patient is likely to be a good responder to the ICI-based therapy.

As illustrated in the experimental part below, the presence of normal levels of Akkermansia muciniphila (Akk^{low}) and/or of Akkermansia SGB9228 (Akk.SGB9228^{low}) in the gut can be considered as a surrogate of host intestinal fitness and immune cell invaded microenvironment, thereby identifying patients who will likely respond to an ICI treatment, whereas Akkermansia muciniphila and/or of Akkermansia SGB9228 indicates dismal prognosis at supraphysiological levels that may reflect intestinal wound healing induced by ATB or other noxious factors. Thus, both the absence and the overrepresentation of Akkermansia muciniphila and/or of Akkermansia SGB9228 in the gut microbiome are the hallmarks of situations in which the patient should receive a compensatory microbial treatment prior to beginning the ICI-based treatment, to improve his/her chance to respond to said ICI-based treatment.

The present invention thus also pertains to a method for determining if a cancer patient needs a bacterial compensation before administration of an ICI-based therapy, comprising measuring, in a sample from said patient, the relative abundance of Akkermansia, wherein:
(i) if Akkermansia is absent in the sample, the patient needs a bacterial compensation with at least Akkermansia before ICI administration;
(ii) if Akkermansia is present in the sample below a predetermined threshold, the patient does not need any bacterial compensation before ICI administration; and
(iii) if Akkermansia is present in the sample above a predetermined threshold, especially if this overrepresentation is consecutive to antibiotics exposure and/or associated with an overrespresentation of species belonging to the Gammaproteobacteria class and/or to the Desulfovibrionaceae family, the patient needs a bacterial compensation with a polymicrobial consortium (especially, a complex polymicrobial consortium, i.e., a consortium of at least 5, preferably at least 7 and for example more than 10 different microbial strains) and/or through fecal microbial transplant (FMT) from a healthy individual or from a cancer patient who successfully responded to the ICI-based therapy.

The experiments reported below also show that the intestinal residence of Akkermansia muciniphila is an indirect marker of richness of the gut ecosystem, as shown by the association of Akkermansia muciniphila at a relative abundance within the 75^{th} percentile with the alpha diversity (Shannon diversity index) of the stools. As a result, the level of Akkermansia muciniphila can be measured to quickly and easily identify gut dysbiosis, which is very useful for all microbiota-centered interventions.

Another aspect of the present invention is thus a method for determining if an individual has an intestinal microbiota dysbiosis, comprising measuring, in a sample from said individual, the relative abundance of Akkermansia, especially of Akkermansia muciniphila and/or Akkermansia SGB9228, wherein the presence of said Akkermansia below a predetermined threshold is indicative that there is no intestinal microbiota dysbiosis, and the absence of bacteria of the Akkermansia genus, especially of Akkermansia muciniphila and/or Akkermansia SGB9228, or their presence above the predetermined threshold, indicate intestinal microbiota dysbiosis.

The present invention also pertains to the use of polymicrobial consortia or FMT, especially with material from healthy individuals or from a cancer patient who successfully responded to the ICI-based therapy, for treating a cancer patient having an overrepresentation of the Akkermansia genus, especially of Akkermansia muciniphila and/or Akkermansia SGB9228 in his/her intestinal microbiota (prior to and in combination with the ICI-treatment).

Another aspect of the invention is a bacterial composition comprising bacteria of the Akkermansia genus, especially of Akkermansia SGB9228 and/or Akkermansia muciniphila, for treating a cancer patient having no Akkermansia muciniphila and no Akkermansia SGB9228 in his/her intestinal microbiota (prior to and in combination with the ICI-treatment). More specifically, therapeutic supplementation with a lyophilized encapsulated strain of Akkermansia SGB9228, such as Akksp2261, benefits the patients not exposed to ATB and devoid of endogenous Akkermansia muciniphila and of endogenous Akkermansia SGB9228, especially when it shifts the microbiome towards favorable health-related species (i.e Intestinimonas butyriciproducens, Odoribacter splanchnicus, Parasuterrella excrementihominis, Roseburia faecis).

### BRIEF DESCRIPTION OF THE DRAWINGS

FIG. 1. Stool A. muciniphila (Akk) is associated with ICI clinical benefit.

A-B Correlations between stool Akk and ORR in 1L+2L (n=338) and 1L immunotherapy NSCLC (n=86) patients. CR; complete response. PR; partial response, SD; stable disease, PD; progressive disease analyzed using Chi-square test. P-values are two-sided, with no adjustments made for multiple comparisons. C-D Kaplan-Meier curves and Cox regression analyses of overall survival (OS) of 1L+2L (n=338) and 2L (n=243) according to Akk status. Akk status was compared using the stratified log-rank test. P-values are one-sided with no adjustment. E. Difference of the intestinal prevalence of Akk between patients with OS<12 months versus >12 months in 1L immunotherapy (10) analyzed using Chi-square test. P-values are two-sided, with no adjustments made for multiple comparisons. F-H. RNA sequencing of tumor biopsies in a sub-group of 44 NSCLC patients (17 non-metastatic and 27 metastatic patients, Table 6). F. Principal Component Analysis (PCoA) of the differentially expressed genes according to intestinal prevalence of Akk, using the 395 immune-related gene selection of the Oncomine Immune Response Research Assay indicating significant differences using a Mann-Whitney p-value<0.10 through PERMANOVA test using Euclidian distance. G. Heatmap of the differentially expressed gene products after normalization (between Akk+vs Akk− patients) classified by category. H. Boxplot of selected gene expression values according to Akk groups: Akk+(n=22) and Akk− (n=22) patients (Table 6). Differences between groups were assessed with Mann-Whitney tests. C—X—C motif chemokine ligand 10 (CXCL10), C—X—C motif chemokine ligand 9 (CXCL9), Guanylate binding protein 1 (GBP1), Vascular cell adhesion protein 1 (VCAM1), C-C chemokine receptor type 5 (CCR5), Granzyme H (GZMH). The middle line of the box represents the median. The lower and upper hinges correspond to the first and third quartiles (the 25th and 75th percentiles). The upper whisker extends from the hinge to the largest value no further than 1.5*IQR from the hinge (where IQR is the inter-quartile range, or distance between the first and third quartiles). The lower whisker extends from the hinge to the smallest value at most 1.5*IQR of the hinge. Data beyond the end of the whiskers are called “outlying” points and are plotted individually.

FIG. 2. Akk relative abundance represents a prognostic marker of ICI.

A. Beta-diversity measured by Bray-Curtis Index represented by Principal Coordinates analysis (PCoA) between Akk⁻ versus Akk⁺ groups between OS< or ≥12 months within each subgroup. OS; overall survival. p-values were calculated using PERMANOVA with 999 permutations. B. Kernel density estimation aligning two variables, OS< or ≥12 and relative abundance of Akk in the entire cohort of 338 patients. The test used was the two-sided Fisher exact test on a 2×2 contingency table. No adjustments were required for this test. C. Distribution of the relative abundance of Akk in patients with detectable Akk according to 77^{th} percentile (left panel) and percentages of patients within each of the three groups of Akk relative abundance (right panel). Akk⁻: undetectable Akk, Akk^{low}: Akk relative abundance between 0.035-4.799%, Akk^{high}: >4.799% (77th percentile). The lower and upper hinges of boxplots correspond to the 25th and 75th percentiles, respectively. The midline is the median. The upper and lower whiskers extend from the hinges to the largest (or smallest) value no further than ×1.5 interquartile range from the hinge, defined as the distance between the 25th and 75th percentiles. D-F. Kaplan-Meier curve and Cox regression multivariate analysis of overall survival in 338 NSCLC patients according to Akk relative abundance segregated in 3 groups (Akk⁻, Akk^{low} and Akk^{high}) (D) and considering PD-L1 expression (F). Akk status was compared using the stratified log-rank test. P-values are one-sided with no adjustment. Cox logistic regression multivariate analysis of overall survival in 338 NSCLC patients according to Akk relative abundance segregated in 3 groups (Akk⁻, Akk^{low} and Akk^{high}) and all the other relevant clinical parameters (E). P-values were calculated using the Wald test including all covariates in the Cox Proportional Hazards Regression Model. Exact P-values are in Table 4. OS: overall survival. ECOG; eastern cooperative oncology group performance status. ATB: antibiotics. G. Distribution of patients according to Akk relative abundance segregated in 2 groups (Akk^{low} and Akk^{high}) and ATB use (noATB: no exposure to ATB, ATB: antibiotics exposure within 2 months prior to ICI initiation). H. Kaplan-Meier curve and Cox regression multivariate analysis of overall survival in 338 NSCLC patients according to Akk relative abundance segregated in 3 groups (Akk⁻, Akk^{low} and Akk^{high}) and ATB use (noATB n=269, top panel and ATB n=69, bottom panel). Akk status was compared using the stratified log-rank test. P-values are one-sided with no adjustment.

FIG. 3. Stratification of clinical outcome based on other components of the Akk-associated ecosystem.

A. Volcano plot (indicating Fold Change (FC) and p-values in Maaslin2 statistical analyses) to segregate taxonomic species (with a prevalence>2.5%) according to their relative abundance in baseline fecal specimen of 338 patients based on their association with Akk: species significantly associated with or excluded from Akk⁻ enriched ecosystems (Akk⁺, dark dots, Akk⁻, underlined). P-values were calculated testing the null hypothesis and using a two-sides test. B, D, F. Kaplan Meier overall survival curves in 338 NSCLC patients according to the trichotomic distribution of the relative abundance of beneficial or harmful bacteria (undetectable bacterium: ⁻, ^{low} versus ^{high}) retained in the LEfSe model, MaAsLin2 and the Volcano plot/ANOVA Table 7). The the trichotomic distribution was compared using the stratified log-rank test. P-values are one-sided with no adjustment. C, E, G. Influence of collateral bacteria associated with Akk (retained in Table 8) in the Akk-associated impact on ORR and OS in a dichotomic pattern (presence/absence) using Chi-square test (for ORR, left panels, P-values are two-sided, with no adjustments made for multiple comparisons). Cox regression multivariate analysis for Kaplan Meier curves (right panels, The the trichotomic distribution was compared using the stratified log-rank test. P-values are one-sided with no adjustment). CR; complete response. PR; partial response, SD; stable disease, PD; progressive disease.

FIG. 4. Consort diagram describing the stool collection in the whole NSCLC ONCOBIOTICS cohort.

Consortium diagram for patients enrollment in the ONCOBIOTICS study (n=493) according to stool availability, presence of Akkermansia muciniphila (Akk), and tumor PD-L1 expression levels. V1; baseline fecal sample. V2; before the second injection of the immune checkpoint inhibitor; MGS: Metagenomic Sequencing. Akk⁺: detection of Akk; Akk⁻: no detection of Akk by shotgun metagaenomics sequencing (MGS) analysis at diagnosis.

FIG. 5. MetaOMineR-based analysis of the association between stool A. muciniphila (Akk) and clinical benefit to ICI in patients.

A-B. Correlations between stool prevalence of Akk (MetaOMineR pipeline) and ORR (A) or OS (B) in 1+2L NSCLC patients (N=338, A-B) based on MGS identification of Akk in the MetaOMineR algorithm (INRAE). Chi-square test (A) and Cox regression analysis for median overall survival (OS) depicted in Kaplan Meier curves according to detectable or undetectable Akk (Akk⁺ or Akk) analyzed in 2 groups (B, left panel) or segregated in 3 groups (Akk⁻, Akk^{low} and Akk^{high}) (B, right panel). Chi-square test P-values are two-sided, with no adjustments made for multiple comparisons (A). The Akk status was compared using the stratified log-rank test. P-values are one-sided with no adjustment (B). C. Experimental setting of avatar mice. FMT of NSCLC patients (Table 6) segregated according to the presence or absence of Akk into MCA-205 tumor bearing C57BL/6 mice. Treatments are indicated by arrows (ATB, FMT, anti-PD-1 (ICI) mAbs, or isotype control mAbs (Iso)). D. MCA-205 tumor growth kinetics in each group of FMT according to the prevalence of Akk. in isotype Ctl versus anti-PD-1 mAbs treated mice. Data are presented as mean values+/−SEM of tumor sizes within 6 animals/group. Concatenation of at least n=8 experiments (using a different stool of NSCLC patient) containing 6 mice/group. Tumor sizes according to FMT Akk⁻ (D, left panel) versus Akk+(D, right panel) are depicted, each dot representing one mouse. Statistics were mixed-effect modeling with specific software ((https://kroemerlab.shinyapps.io/TumGrowth/) for longitudinal tumor growth analysis. P-value are indicated. E. Percentages of responding mice (tumor reduction of >25% compared with means of controls in the anti-PD-1 mAbs-treated group) and patients (ORR) in each category of stools used for FMT (derived from patients in Table 6). CR; complete response. PR; partial response, SD; stable disease, PD; progressive disease.

FIG. 6. Metagenomic species characterizing Akk⁺ stools and patient survival.

Shannon diversity index representing stool alpha diversity in Akk⁺ and Akk⁻ groups of fecal specimens (N=338) (A, upper panel). Beta-diversity measured by Bray-Curtis Index represented by Principal Coordinates analysis (PCoA) between Akk⁺ versus Akk⁻ groups in the whole cohort of 1+2L (A, lower panel). p-values were calculated using PERMANOVA with 999 permutations. The lower and the upper hinges of boxplots corresponds to the 25^{th} and 75^{th} percentiles, respectively. The midline is the median. The upper and lower whiskers extend from the hinges to the largest (or smallest) value no further than ×1.5 interquartile range from the hinge, defined as the distance between the 25^{th} and 75^{th} percentiles. P-values were calculated testing the null hypothesis and using a two-sided test. Exact p-value: 3.84573e-05. B-C. Differential abundance of metagenomic species measured by linear discriminant analysis of effect size (LEfSe) according to the presence of A. muciniphila (Akk) (B) and the OS at 12 months (C) within Akk+ group (C, left panel) and Akk− group (C, right panel). LDA; Linear discriminant analysis. OS: overall survival. P-values were calculated using a two-sided nonparametric factorial Kruskal-Wallis (KW) sum-rank test. #Multivariate analysis (ANCOM-BC/Maaslin2) with a false discovery rate (FDR) adjusted p-value<0.2.

FIG. 7. Compositional taxonomic differences in stools of NSCLC patients segregated according to Akk relative abundance.

A. Alpha diversity according to Akk relative abundance segregated in 3 groups Akk⁻: undetectable Akk, Akk^{low}: A. muciniphila relative abundance between 0.035-4.799% (<77^{th} percentile of positive samples), and Akk^{high}: 4.799% (>77^{th} percentile) (N=338). The lower and upper hinges of boxplots correspond to the 25th and 75th percentiles, respectively. The midline is the median. The upper and lower whiskers extend from the hinges to the largest (or smallest) value no further than ×1.5 interquartile range from the hinge, defined as the distance between the 25th and 75th percentiles. P-values were calculated using a two-sided nonparametric Wilcoxon sum-rank test. B-C. Beta-diversity using PCoA between Akk⁻ and Akk^{low}(B) and between Akk^{low} and Akk^{high} (C) p-values were calculated using PERMANOVA with 999 permutations. The PERMANOVA test compares groups of objects and tests the null hypothesis that the centroids and dispersion of the groups are equivalent. The P-value is calculated by comparing the actual F test to that gained from (in this case 999) random permutations of the objects between the groups. If p<0.05, the null hypothesis is disregarded and we conclude that the centroids and dispersion between the groups are not equivalent. D-E. Variable importance plot (VIP) discriminant analysis of taxonomic stool composition according to Akk relative abundance, between Akk⁻ versus Akk^{low} (D) and Akk^{low} versus Akk^{high} (E). Differences in bacterial prevalence and abundance in fold ratios are indicated in these VIP plots. VIP: Variable importance plot. * p<0.05,** p<0.01, *** p<0.001. P-values were calculated using a two-sided nonparametric Wilcoxon sum-rank test. #Multivariate analysis (ANCOM-BC/Maaslin2) with a false discovery rate (FDR) adjusted p-value<0.2

FIG. 8. Interaction between ATB and A. muciniphila on survival and microbiome composition.

A. Kaplan-Meier curve and Cox regression analysis of overall survival in the n=338 patients according to detectable versus undetectable Akk (Akk⁺ and Akk⁻) and ATB use (noATB: no exposure to ATB, ATB: antibiotics exposure within 2 months prior to ICI initiation). The Akk status and ATB use were compared using the stratified log-rank test. P-values are one-sided with no adjustment. B. Shannon diversity index representing stool alpha diversity in Akk⁺ and Akk⁻ groups of fecal specimen from patients exposed or not to ATB (N=338). The lower and upper hinges of boxplots correspond to the 25th and 75th percentiles, respectively. The midline is the median. The upper and lower whiskers extend from the hinges to the largest (or smallest) value no further than ×1.5 interquartile range from the hinge, defined as the distance between the 25th and 75th percentiles. P-values were calculated using a two-sided nonparametric Wilcoxon sum-rank test. C. Box Plots representing the relative abundance (mean+/−SEM) of Akk according to overall survival at 12 months and exposure or not to ATB in n=338 patients. The lower and upper hinges of boxplots correspond to the 25th and 75th percentiles, respectively. The midline is the median. The upper and lower whiskers extend from the hinges to the largest (or smallest) value no further than ×1.5 interquartile range from the hinge, defined as the distance between the 25th and 75th percentiles. The test used was Kruskal-Wallis, two-sided, 5% level of significance. No adjustments were made for multiple comparisons.

FIG. 9. Akkermansia p2261 modulated the murine microbiome composition, rescuing responsiveness to PD-1 blockade.

A. Experimental setting. After 3 days of ATB, FMT was performed in mice by oral gavage using patient stools classified according to Akk (Akk⁺ and Akk⁻). 14 days later, MCA-205 tumors were i.d inoculated, and mice were treated with anti-PD-1 or iso-control mAbs 4 times every 3 days concomitantly with oral supplementation of Akkermansia p2261 four times every 3 days. B-D. Mean MCA-205 tumor sizes+/−SEM are depicted at day 12 after 4 therapeutic injections of anti-PD-1 mAbs, in each FMT groups (Akk⁺ and Akk⁻) supplemented or not with Akkermansia p2261 as well as in animals reared in SPF conditions (FMT−). Concatenation of >25 experiments using n=53 mice in Iso group, n=51 in Iso FMT+ group, n=56 in anti-PD-1 and anti-PD-1 FMT+ groups. Each experiment comprising 6 mice/group and was performed at least 2 times for each FMT (Table 7) (B). Tumor sizes according to FMT Akk⁻ (C left, n=72/group; C right, n=49 in Iso group and n=48 in other groups) versus Akk⁺ (D top, n=6/group, D bottom, n=12 in Iso and anti-PD-1 groups, n=14 in anti-PD-1 with Akkermansia p2261) are depicted, each dot representing one mouse. Statistics were mixed-effect modeling with specific software ((https://kroemerlab.shinyapps.io/TumGrowth/) for longitudinal tumor growth analysis (D) and Mann-Whitney U-test (B-C) to compare two independent groups (after Kruskal-Wallis test was implemented using Dunn's test for multiple groups). ns=not significant. E. Clustermap of ratios of Akkp2261-related tumor reduction at day 12-15 following PD-1 mAbs in FMT normalized onto ratios obtained in SPF mice. The relative tumor size reduction follows a grey color code (the darker the greater; R, Responders)). 29 FMT were performed according to A. N=29-30 mice/group in total. Each experiment contained 6 mice/group and was performed 2-3 times for each tumor model (E, left panel). 16S rRNA sequencing of gene amplicons of stools harvested in recipient avatar tumor bearers at day 12 post-4 injections of anti-PD-1 Abs and 4 oral gavages with Akkermansia p2261 divided into light grey (R) and dark grey (NR) groups. VIP plot repartition of discriminant metagenomic species segregating groups of mice that responded to oral Akkermansia p2261 (R, light grey bars) or not (NR, dark grey bars). (E, right panel). Asterisks represent significant Mann-Whitney U test without FDR at 10%. * p<0.05, ** p<0.01, *** p<0.001. P-values were calculated using a two-sided nonparametric Wilcoxon sum-rank test. Adjustments for multiple comparisons were not made.

FIG. 10. Akk relative abundance represents a prognostic marker of ICI. Kaplan-Meier curve of overall survival in NSCLC patients according to Akk relative abundance segregated in 3 groups (Akk⁻, Akk^{low} and Akk^{high}) and two clades SGB9226 (as a proxy of A. muciniphila) and SGB9228.

FIG. 11. Effect of strains of Akkermansia on the therapeutic response to checkpoint blockade in SPF mice versus mice having received FMT from non-responder patients. Tumor size shown for ATB-treated mice receiving fecal microbial transplantation from one non-responder patient alone followed by inoculation of MCA-205 sarcomas and PD-1 blockade (n=6 per group). A representative experiment is shown. Compensation of dysbiosis was attempted by addition of Akkermansia strain 2261, 5801, 5126, 4531 or 3284. Anova statistics *p<0.05, **p<0.01, ***p<0.001.

FIG. 12: IL-12 production by bone marrow-derived dendritic cells (BM-DC) exposed to several commensals (MOI 1:20). Bone marrow-dendritic cells were obtained by in vitro culture in GM-CSF/IL-4 and were stimulated for 1 hr with live strains of Akkermansia (aligned in the x axis), which were subsequently neutralized by appropriate ATB, prior to harvesting supernatants at 24 hrs to monitor IL-12p70 concentrations by commercial ELISA. Anova statistics *p<0.05, **p<0.01, ***p<0.001.

FIG. 13: qPCR-based identification of Akkermansia mucinphila strains (5801, 5126 and 5145) or Akkermansia SGB9228 strains (4531 and 2261) using primers recognizing either both species of Akkermansia (SGB9226/9228, top left panel), Akkermansia muciniphila strains (Akkermansia SGB9226, top right panel), Akkermansia SGB9228 strains (bottom left panel), or primers specific for the p2261 strain (bottom right panel).

### DETAILED DESCRIPTION OF THE PREFERRED EMBODIMENTS

In the present text, the following general definitions are used:

### Gut Microbiota

The “gut microbiota” (formerly called gut flora or microflora) designates the population of microorganisms living in the intestine of any organism belonging to the animal kingdom (human, animal, insect, etc.). While each individual has a unique microbiota composition (60 to 80 bacterial species are shared by more than 50% of a sampled population on a total of 400-500 different bacterial species/individual), it always fulfils similar main physiological functions and has a direct impact on the individual's health:
it contributes to the digestion of certain foods that the stomach and small intestine are not able to digest (mainly non-digestible fibers);
it contributes to the production of some vitamins (B and K);
it protects against aggressions from other microorganisms, maintaining the integrity of the intestinal mucosa;
it plays an important role in the development of a proper immune system;
a healthy, diverse and balanced gut microbiota is key to ensuring proper intestinal functioning.

Taking into account the major role gut microbiota plays in the normal functioning of the body and the different functions it accomplishes, it is nowadays considered as an “organ”. However, it is an “acquired” organ, as babies are born sterile; that is, intestine colonisation starts right after birth and evolves afterwards.

The development of gut microbiota starts at birth. Sterile inside the uterus, the newborn's digestive tract is quickly colonized by microorganisms from the mother (vaginal, skin, breast, etc.), the environment in which the delivery takes place, the air, etc. From the third day, the composition of the intestinal microbiota is directly dependent on how the infant is fed: breastfed babies' gut microbiota, for example, is mainly dominated by Bifidobacteria, compared to babies nourished with infant formulas.

The composition of the gut microbiota evolves throughout the entire life, from birth to old age, and is the result of different environmental influences. Gut microbiota's balance can be affected during the ageing process and, consequently, the elderly have substantially different microbiota than younger adults.

While the general composition of the dominant intestinal microbiota is similar in most healthy people (4 main phyla, i.e., Firmicutes, Bacteroidetes, Actinobacteria and Proteobacteria), composition at a species level is highly personalised and largely determined by the individuals' genetic, environment and diet. The composition of gut microbiota may become accustomed to dietary components, either temporarily or permanently. Japanese people, for example, can digest seaweeds (part of their daily diet) thanks to specific enzymes that their microbiota has acquired from marine bacteria.

### Dysbiosis

Although it can adapt to change and has a high resilience capacity, a loss of balance in gut microbiota composition may arise in some specific situations. This is called “dysbiosis”, a disequilibrium between potentially “detrimental” and “beneficial” bacteria in the gut or any deviation to what is considered a “healthy” microbiota in terms of main bacterial groups composition and diversity. Dysbiosis may be linked to health problems such as functional bowel disorders, inflammatory bowel diseases, allergies, obesity and diabetes. It can also be the consequence of a treatment, such as a cytotoxic treatment or an antibiotic treatment.

### Immune Checkpoint Blockers

In the present text, an “immune checkpoint inhibitor”, or “ICI”, a “drug blocking an immune checkpoint”, or “immune checkpoint blocker” or “immune checkpoint blockade drug” designates any drug, molecule or composition which blocks an immune checkpoint. In particular, it encompasses anti-PD1 antibodies, anti-PD-L1 antibodies (such as Atezolizumab or Durvalumab), anti-CTLA-4 antibodies and anti-PD-L2 antibodies. More particularly, it can be an anti-PD1 monoclonal antibody such as Nivolumab or Pembrolizumab.

An “anti-PD1/PD-L1 Ab-based therapy” herein designates any drug that antagonizes PD1 or PD-L1. Although the currently used drugs antagonizing PD1 or PD-L1 are monoclonal antibodies, other molecules specifically binding to PD1, PD-L1 could be used for the development of future ICI such as, for example, antibody fragments or specifically designed aptamers. Of course, the phrase “anti-PD1/PD-L1 Ab-based therapy” encompasses any therapy with active molecules that antagonize PD1 or PD-L1.

### Cancer, Treatment, Etc.

As used herein, “cancer” means all types of cancers. In particular, the cancers can be solid or non solid cancers. Non limitative examples of cancers are carcinomas or adenocarcinomas such as breast, prostate, ovary, lung, pancreas or colon cancer, sarcomas, lymphomas, melanomas, leukemias, germ cell cancers and blastomas.

The immune system plays a dual role against cancer: it prevents tumor cell outgrowth and also sculpts the immunogenicity of the tumor cells. Drugs blocking an immune checkpoint can hence be used to treat virtually any type of cancer. Thus, the methods according to the invention are potentially useful for patients having a cancer selected amongst adrenal cortical cancer, anal cancer, bile duct cancer (e.g. periphilar cancer, distal bile duct cancer, intrahepatic bile duct cancer), bladder cancer, bone cancers (e.g. osteoblastoma, osteochrondroma, hemangioma, chondromyxoid fibroma, osteosarcoma, chondrosarcoma, fibrosarcoma, malignant fibrous histiocytoma, giant cell tumor of the bone, chordoma, lymphoma, multiple myeloma), brain and central nervous system cancers (e.g. meningioma, astocytoma, oligodendrogliomas, ependymoma, gliomas, medulloblastoma, ganglioglioma, Schwannoma, germinoma, craniopharyngioma), breast cancer (e.g. ductal carcinoma in situ, infiltrating ductal carcinoma, infiltrating lobular carcinoma, lobular carcinoma in situ, gynecomastia), Castleman disease (e.g. giant lymph node hyperplasia, angiofollicular lymph node hyperplasia), cervical cancer, colorectal cancer, endometrial cancers (e.g. endometrial adenocarcinoma, adenocanthoma, papillary serous adenocarcinoma, clear cell), esophagus cancer, gallbladder cancer (mucinous adenocarcinoma, small cell carcinoma), gastrointestinal carcinoid tumors (e.g. choriocarcinoma, chorioadenoma destruens), Hodgkin's disease, non-Hodgkin's lymphoma, Kaposi's sarcoma, kidney cancer (e.g. renal cell cancer), laryngeal and hypopharyngeal cancer, liver cancers (e.g. hemangioma, hepatic-adenoma, focal nodular hyperplasia, hepatocellular carcinoma), lung cancers (e.g. small cell lung cancer, non-small cell lung cancer), mesothelioma, plasmacytoma, nasal cavity and paranasal sinus cancer (e.g. esthesioneuroblastoma, midline granuloma), nasopharyngeal cancer, neuroblastoma, oral cavity and oropharyngeal cancer, ovarian cancer, pancreatic cancer, penile cancer, pituitary cancer, prostate cancer, retinoblastoma, rhabdomyosarcoma (e.g. embryonal rhabdomyosarcoma, alveolar rhabdomyosarcoma, pleomorphic rhabdomyosarcoma), salivary gland cancer, skin cancer (e.g. melanoma, nonmelanoma skin cancer), stomach cancer, testicular cancers (e.g. seminoma, nonseminoma germ cell cancer), thymus cancer, thyroid cancers (e.g. follicular carcinoma, anaplastic carcinoma, poorly differentiated carcinoma, medullary thyroid carcinoma, thyroid lymphoma), vaginal cancer, vulvar cancer, and uterine cancer (e.g. uterine leiomyosarcoma). More particularly, the method according to the invention can be used for predicting and optimizing a patient's response to a medicament targeting an immune checkpoint, wherein the patient has a cancer selected from the group consisting of metastatic melanoma, non-small cell lung carcinoma (NSCLC), small cell lung cancer (SCLC), mesothelioma, bladder cancer, renal cell carcinoma, head and neck cancers, oesophageal and gastric cancers, rectal cancers, hepatocarcinoma, sarcoma, Wilm's tumor, Hodgkin lymphoma, ALK-neuroblastoma, (hormone refractory) prostate cancers and GIST.

Other definitions will be specified below, when necessary.

According to a first aspect, the present invention pertains to an in vitro theranostic method of determining if a cancer patient is likely to be a good responder to an immune checkpoint inhibitor (ICI)-based therapy, comprising measuring, in a sample from said patient, the relative abundance of Akkermansia muciniphila and/or Akkermansia SGB9228, wherein the presence of Akkermansia muciniphila and/or Akkermansia SGB9228 below a predetermined threshold (“superior threshold”) is indicative that the patient is likely to be a good responder to the ICI-based therapy.

Akkermansia muciniphila and Akkermansia SGB9228 are the two most prevalent Akkermansia species and can be detected together using, for example, primers hybridizing to genes common to all Akkermansia. According to another embodiment, the present invention thus relates to an in vitro theranostic method of determining if a cancer patient is likely to be a good responder to an immune checkpoint inhibitor (ICI)-based therapy, comprising measuring, in a sample from said patient, the relative abundance of the Akkermansia genus, wherein the presence of bacteria of the Akkermansia genus below a predetermined threshold (“superior threshold”) is indicative that the patient is likely to be a good responder to the ICI-based therapy.

In WO2018115519, the inventors already showed that the absence of Akkermansia muciniphila in a feces sample from a cancer patient is indicative of a resistance to PD1 blockade. In the experiments reported below, they now demonstrated that the overrepresentation of Akkermansia muciniphila and/or Akkermansia SGB9228 in a feces sample, i.e., its presence above a superior threshold, is indicative of dismal prognosis despite ICI-treatment. As shown at least in FIG. 2D, the presence of Akkermansia muciniphila at supraphysiologic levels correlates with an overall survival significantly lower than the overall survival of patients whose feces is bereft of Akkermansia muciniphila, itself lower that the overall survival of patients exhibiting Akkermansia muciniphila at a level comprised between an inferior threshold (corresponding to the detection limit in the reported experiments) and a superior threshold, hereafter designated as the “predetermined threshold”.

An example of threshold that can be used as “predetermined threshold” in the frame of the invention is disclosed in the experimental part below. Of course, the skilled artisan can adapt or refine this threshold, depending on the technique used to measure the relative abundance of Akkermansia muciniphila and/or Akkermansia SGB9228 and/or of the Akkermansia genus (for example, metagenomics, quantitative PCR, hybridization on a microarray or pyrosequencing), the species of Akkermansia which is(are) detected, the specific pathology of the patient, the patient's food habits, the specific ICI used for the treatment and other possible factors. For example, the threshold to be considered when performing the above method can be predetermined by measuring the relative abundance of Akkermansia muciniphila and/or Akkermansia SGB9228, and/or of the Akkermansia genus in a representative cohort of individuals having the same cancer as the patient for whom a prognostic is sought, and choosing as threshold the value of the 75^{th} percentile. This threshold can be different for Akkermansia muciniphila and for Akkermansia SGB9228.

For illustrative purpose, the values of the relative abundances of Akkermansia muciniphila obtained in healthy volunteers (from available literature) and from three cohorts of metastatic patients diagnosed with melanoma or with kidney or lung cancers are shown below.

TABLE 1
relative abundances of Akkermansia muciniphila in different cohorts

| class | mean | median | min | max | quantile75 | N |
|---|---|---|---|---|---|---|
| healthy | 2.309326122 | 0.53485 | 6.00E−05 | 80.52095 | 2.2534075 | 3244 |
| kidney | 3.037814 | 0.69659 | 0.00061 | 21.35266 | 3.83326 | 45 |
| lung | 4.521102941 | 1.0551 | 0.00163 | 71.72794 | 6.0443025 | 204 |
| melanoma | 3.0422872 | 0.21848 | 0.00012 | 27.46063 | 3.240995 | 75 |

In the present text, the “presence of Akkermansia muciniphila below a predetermined threshold” thus means that Akkermansia muciniphila is present at a level between two thresholds: an inferior threshold (close to 0, typically <0.001, for example 0.0005) and a superior threshold (the “predetermined threshold” as described above). The same applies for Akkermansia SGB9228 and for the Akkermansia genus.

According to a particular embodiment of the present invention, the predetermined threshold corresponds to a relative abundance between 1 and 10%, for example between 3 and 6.5%.

According to another particular embodiment, the predetermined threshold is selected so that it is between the 75^{th} and the 77^{th} percentile. It can be selected by grid search algorithm. With the cohort used in the experimental part below, the selected cutoff (predetermined threshold) corresponds to 4.79 (77th percentile), measurement error 4.75+/−0.1. A predetermined threshold of 4.75+/−0.1 can thus be used as superior threshold when performing the method of the invention. Of course, as already mentioned, this threshold can be refined or adapted by the skilled person, by routine experiments.

According to another particular embodiment of the present invention, the ICI-based therapy is an anti-PD1/PD-L1/PD-L2 Ab-based therapy (such as, but not limited to Nivolumab, Pembrolizumab, Atezolizumab and Durvalumab) or an anti-CTLA4 Ab-based therapy (such as, but not limited to Ipulimumab), or a combination thereof.

As illustrated in the experimental part below, the present invention is particularly useful for patients suffering from non small cell lung cancer (NSCLC) or kidney cancer, or from any cancer amenable to PD1/PDL-1 or CTLA4 blockade.

More generally, the methods of the invention can be advantageously performed for cancer patients who suffer from any cancer amenable to immunotherapy, such as, but not limited to: melanoma; renal cell carcinoma (RCC); Non small-cell lung carcinoma (NSCLC); Head and Neck squamous cell carcinoma (HNSCC); Merkel cell carcinoma (MCC); bladder cancer; Hodgkin lymphoma; squamous cell carcinoma; breast cancer, especially triple-negative breast cancer; gastric cancer; small-cell lung carcinoma; primary mediastinal B-cell lymphoma; cervical cancer; hepatocellular carcinoma; esophageal cancer; cancers with MicroSatellite Instability; endometrial cancer and any cancer with a high tumor mutational burden (TMB-H cancers).

As illustrated in the experimental part below, the present invention is useful in situations where the ICI-based therapy is administered as first-line therapy or second-line therapy or beyond (3^{rd}, 4^{th} line).

According to a particular aspect of the present invention, the predetermined threshold is chosen such that the presence of Akkermansia muciniphila and/or Akkermansia SGB9228, and/or of the Akkermansia genus above this threshold is indicative of dismal prognosis despite ICI-based therapy.

According to another aspect, the present invention pertains to a method for in vitro determining if a cancer patient needs a bacterial compensation before administration of an ICI-based therapy, and to provide the physician with information related to the type of compensation that can improve the patient's likelihood to respond to the treatment.

According to a particular embodiment, this method comprises measuring, in a sample from said patient, the relative abundance of Akkermansia muciniphila and/or Akkermansia SGB9228, and provides the physician with the following guide:
(i) if Akkermansia muciniphila and/or Akkermansia SGB9228 is absent in the sample, the patient needs a bacterial compensation with at least Akkermansia muciniphila and/or Akkermansia SGB9228 before ICI administration;
(ii) if Akkermansia muciniphila and/or Akkermansia SGB9228 is present in the sample below a predetermined threshold, the patient does not need any bacterial compensation before ICI administration; and
(iii) if Akkermansia muciniphila and/or Akkermansia SGB9228 is present in the sample above a predetermined threshold, especially if this overrepresentation is consecutive to antibiotics exposure and/or associated with an overrespresentation of species belonging to the Gammaproteobacteria class and/or to the Desulfovibrionaceae family, the patient needs a bacterial compensation with a complex polymicrobial consortium and/or through fecal microbial transplant (FMT) from a healthy individual or from a cancer patient who successfully responded to the ICI-based therapy.

According to another particular embodiment, the method for in vitro determining if a cancer patient needs a bacterial compensation before administration of an ICI-based therapy comprises measuring, in a sample from said patient, the relative abundance of the Akkermansia genus, and provides the physician with the following guide:
(i) if the Akkermansia genus is absent in the sample, the patient needs a bacterial compensation with bacteria of the Akkermansia genus before ICI administration, for example with bacteria of the candidate species Akkermansia SGB9228, such as the strain deposited at the Collection de souches de l'Unité des Rickettsies (CSUR) under the reference CSUR P2261 or the strain deposited at the CSUR under the reference CSUR 4531;
(ii) if the Akkermansia genus is present in the sample below a predetermined threshold, the patient does not need any bacterial compensation before ICI administration; and
(iii) if the Akkermansia genus is present in the sample above a predetermined threshold, especially if this overrepresentation is consecutive to antibiotics exposure and/or associated with an overrespresentation of species belonging to the Gammaproteobacteria class and/or to the Desulfovibrionaceae family, the patient needs a bacterial compensation with a complex polymicrobial consortium and/or through fecal microbial transplant (FMT) from a healthy individual or from a cancer patient who successfully responded to the ICI-based therapy.

According to a particular embodiment of the above method, the predetermined threshold corresponds to a relative abundance of Akkermansia muciniphila and/or Akkermansia SGB9228 and/or the Akkermansia genus between 1 and 10%, for example between 3 and 6.5% or any other predetermined threshold as described above.

According to another particular embodiment of the above method, the ICI-based therapy is an anti-PD1/PD-L1/PD-L2 Ab-based therapy or an anti-CTLA4 Ab-based therapy or a combination thereof (as described above).

The method according to the present invention is particularly useful for in vitro determining if a cancer patient suffering from non small cell lung cancer (NSCLC), especially from non-squamous NSCLC needs a bacterial compensation before administration of an ICI-based therapy, as well as for in vitro determining if a cancer patient suffering from kidney cancer or from any cancer amenable to PD1/PD-L1/PD-L2 and/or CTLA4 blockade needs a bacterial compensation before administration of an ICI-based therapy.

According to a particular aspect of the above method, the method is performed before an ICI-based therapy administered as first-line therapy, to assess whether the patient needs a bacterial compensation for improving his/her chances of responding to this therapy.

According to another particular aspect of the above method, the method is performed before an ICI-based therapy administered as or second-line therapy or beyond (3^{rd}, 4^{th} line).

The inventors surprisingly demonstrated that the intestinal residence of bacteria of the Akkermansia genus, such as Akkermansia muciniphila and Akkermansia SGB9228, is an indirect marker of richness of the gut ecosystem, as shown by the association of Akkermansia muciniphila at a relative abundance within the 75^{th} percentile with the alpha diversity (Shannon diversity index) of the stools, so that the level of Akkermansia muciniphila and/or Akkermansia SGB9228 and/or the Akkermansia genus can be measured to quickly and easily identify gut dysbiosis.

Another aspect of the present invention, particularly useful for all microbiota-centered interventions, is thus a method for determining if an individual has an intestinal microbiota dysbiosis, comprising measuring, in a sample from said patient, the relative abundance of Akkermansia muciniphila and/or Akkermansia SGB9228 and/or the Akkermansia genus, wherein the presence of Akkermansia muciniphila and/or Akkermansia SGB9228 and/or the Akkermansia genus below a predetermined threshold is indicative that there is no intestinal microbiota dysbiosis.

According to a particular embodiment of the above method, the predetermined threshold corresponds to a relative abundance between 1 and 10%, for example between 3 and 6.5% or any other predetermined threshold as described above.

In the methods according to any of the preceding aspects, the sample from said patient or individual can be a feces sample or a sample from the colon or ileal luminal content of said patient or individual, or a mucosal biopsy from said patient or individual.

According to another of its aspects, the present invention relates to the use of a fecal microbial composition in the treatment of a cancer patient having an overrepresentation of Akkermansia muciniphila and/or Akkermansia SGB9228 and/or the Akkermansia genus in his/her intestinal microbiota, especially to restore a healthy microbiota before administering an ICI-based therapy, to improve the patient's chances of responding to the treatment. Indeed, as illustrated in the experimental part below, the inventors have demonstrated that although the presence of Akkermansia muciniphila and/or Akkermansia SGB9228 predicts favorable clinical outcome when present at levels compatible with homeostasis, an overrepresentation of Akkermansia muciniphila and/or Akkermansia SGB9228 indicates dismal prognosis. This overrepresentation can result from intestinal wound healing induced by ATB or other noxious factors and is indicative of dismal prognosis despite ICI-treatment. According to a particular embodiment, the fecal microbial composition originates from a healthy individual or from a cancer patient who successfully responded to the ICI-based therapy.

As shown in the experimental part below, the inventors could restore responsiveness to PD-1 blockade in a model of avatar mice that were ATB-treated and then received FMT from patients who were doomed to failed therapy. They showed that the best beneficial effect was obtained in experiments where donor Akk was undetectable. According to yet another of its aspects, the present invention thus relates to the use of a bacterial composition comprising Akkermansia muciniphila or Akkermansia SGB9228, in the treatment of a cancer patient having no Akkermansia muciniphila and no Akkermansia SGB9228 in his/her intestinal microbiota, especially to improve the patient's chances of responding to an ICI-based treatment.

According to a particular embodiment of the bacterial composition of the invention, the Akkermansia bacteria are from the strain deposited at the Collection de souches de l'Unite des Rickettsies (CSUR) under the reference CSUR P2261. This strain, initially identified as Akkermansia muciniphila, was recently reclassified in the Akkermansia SGB9228 candidate species.

According to another particular embodiment of the bacterial composition of the invention, the Akkermansia bacteria are from the strain deposited at the Collection de souches de l'Unité des Rickettsies (CSUR) under the reference CSUR 4531.

The fecal microbial composition or the bacterial composition according to the invention can be particularly useful if they are administered before and/or the ICI-based therapy, particularly in combination with a treatment with an anti-PD1/PD-L1 Ab-based therapy or an anti-CTLA4 Ab-based therapy or a combination thereof.

According to a particular embodiment of the invention, the above-described microbial composition or bacterial composition is used in the treatment of a patient who suffers from non small cell lung cancer (NSCLC), especially from non-squamous NSCLC, or from kidney cancer or any cancer amenable to PD1/PDL-1 or CTLA4 blockade, as detailed above.

According to another particular embodiment of the invention, the above-described microbial composition or bacterial composition is used in the treatment of a patient who received an ICI-based therapy as first-line therapy or second-line therapy or beyond.

When performing the above methods, the relative abundances of Akkermansia muciniphila can be measured by quantitative PCR using the following primers:

| Name | Sequence | SEQ ID No. | Specificity |
|---|---|---|---|
| AkkermansiaSGB9226/9228_F | TTCGCAACGGACGAAGTGTA | 1 | A. muciniphila + |
| AkkermansiaSGB9226/9228_R | TCCGTATACGTGTCCCCGAT | 2 | A. SGB9228 |
| AkkermansiaSGB9226_F | CGGAGCCGAAAATACCCAGA | 3 | Akkermansia |
| AkkermansiaSGB9226_R | TCACCTTTGGCAAGTTCATCCT | 4 | muciniphila |
| AkkermansiaSGB9228_F | GGCTGAAAACACCCAGATGC | 5 | Akkermansia |
| AkkermansiaSGB9228_R | TGGCGAGTTCGTCCTTCAAC | 6 | SGB9228 |
| Akker_rpob_F | GCAACAGGGTCTTGGTGATT | 7 | Akkermansia |
| Akker_rpob_R | GCAGCTCATTGACCAGTTGA | 8 | muciniphila |
| p2261 forward | TGCGTATGGTGGGGATATCC | 9 | Akkermansia |
| p2261 reverse | CCTTCAGTCCGTTCTCCACT | 10 | strain P2261 |

Other characteristics of the invention will also become apparent in the course of the description which follows of the biological assays which have been performed in the framework of the invention and which provide it with the required experimental support, without limiting its scope.

### EXAMPLES

### Example 1: Intestinal Akkermansia muciniphila Predicts Clinical Response to PD1 Blockade in Advanced Non-Small Cell Lung Cancer Patients

### Methods

### Study Design and Treatment

Ethical issues. The ancillary studies have been designed according to an IRB approved-study (Oncobiotics* Sponsor Protocol N: Center for Security and Emerging Technology (CSET) 2017/2619, Agence nationale de sécurité du médicament et des produits de santé ID-RCB N: 2017-A02010-53 https://clinicaltrials.gov/ct2/show/NCT04567446). The trial was conducted in accordance with Good Clinical Practice guidelines and the provisions of the Declaration of Helsinki. All patients provided written informed consent. General Data Protection Regulation procedures and anonymization rules have been applied according to Oncobiome H2020 model system already in place in the ClinicoBiome, Gustave Roussy. All data and sample collection were performed in compliance with regulatory, ethical, and European GDPR requirements.

Patients eligibility. NCT04567446, a multicentric prospective observational study designed to evaluate the impact of the microbiome composition in the clinical outcome of advanced NSCLC patients treated with anti-PD-(L)1. We enrolled across 12 academic centers in France and two in Canada. Adult patients with pathologically confirmed advanced non-squamous or squamous NSCLC and an Eastern Cooperative Oncology Group (ECOG) performance-status score of 0-2, amenable to ICI as standard-of-care and compelling to provide a stool sample were eligible. Eligible patients received ICI following progression on platinum-based chemotherapy regimens, either with nivolumab or atezolizumab regardless of PD-L1 expression or with pembrolizumab if PD-L1≥1%. Given the subsequent approval of first-line ICI in the first-line setting during the study accrual period, patients who received pembrolizumab monotherapy or in combination with platinum-based chemotherapy, depending on PD-L1 expression were also included. Standard-of-care treatment was continued until disease progression, unacceptable adverse effects, or completion as per protocol (2 years of ICI). Full eligibility criteria are listed in the trial protocol (NCT04567446). Baseline characteristics including detailed listing of concurrent medications received the last two months prior to ICI initiation, and date of last follow-up were entered at each center in an electronic case report form.

Hypothesis. Sample size calculation was performed based on the primary end-point defined as investigator-assessed ORR from the hypothesis proposed in Routy et al (Routy, Le Chatelier, et al. 2018) that in a population with metagenomics detectable Akk (Akk⁺) in the gut microbiome, the response rate would be higher than in the population with undetectable Akk (Akk⁻). We considered that a meaningful clinical difference would correlate to an ORR incremental from 10% in the Akk⁻ to 20% in the Akk⁺ group. Given the superiority hypothesis, power was set at 80% with a two-sided alpha level of 5%, using EAST® program. Hence, we determined that at least 292 patients would be necessary to confirm our primary objective.

Study end-point and assessments. Computed tomography scans were performed at baseline and every 8-12 weeks for the first year and every 12-15 weeks thereafter until disease progression. Tumor response was assessed using the Response Evaluation Criteria in Solid Tumors version (RECIST) 1.1 (Eisenhauer et al. 2009). The primary end-point was investigator-assessed objective response rate (ORR) which was defined as the number and percentage of patients with a Best Overall Response (BOR) of confirmed complete response (CR) or partial response (PR). Best overall response (BOR) was defined as the best response designation, recorded between the date of first treatment dose and the date of the initial objectively documented tumor progression per RECIST v1.1 or the date of subsequent therapy, whichever occurs first. For patients without documented progression or subsequent therapy, all available response designations contributed to the BOR determination. Secondary end-points included overall survival (OS), and microbiome variables such as alpha and beta diversity and differential abundance analyses at the genus-level. Overall survival was defined as the time from trial inclusion until death from any cause. The follow-up of patients alive at the database lock was censored to the date of last record of contact.

Treatment modalities: the number of Pembrolizumab (every other 21 days) or Nivolumab (every other 15 days) or Atezolizumab (every other 21 days) injections received was 4+/−2 at 8-12 weeks and was 20+/−4 at 12 months.

### Human Stool Samples and Metagenomics Analyses

Fecal samples were prospectively collected (V1: pre-ICI, V2: before the second ICI injection, V3: at 3 months post-ICI and V4: at 6 months post-ICI) at each center following the International Human Microbiome Standards (IHMS) guidelines. Only the baseline V1 sample was considered for this analysis and for patients where such timely collection was not feasible, V2 samples were considered “baseline” as in (1). For metagenomic analysis, the stools were processed for total DNA extraction and sequencing with Ion Proton technology following MetaGenoPolis (INRA) France, as previously reported (Routy, Le Chatelier, et al. 2018; Li et al. 2014; Nielsen et al. 2014). Cleaning, filtering and classification of reads were performed with two different pipelines: MetaOMineR and MetaPhlAn 3 (Beghini et al. 2020, 3). In order to determine Akkermansia muciniphila presence/absence, we used a total of 463 genetic markers identified from four Akkermansia candidate species-level genome bins (SGBs) (SGB9223—38 markers, SGB9224—54 markers, SGB9226—171 markers and SGB9228—200 markers)(Karcher et al. 2021) in MetaPhlAn. As outlined in the Table 2, the type of strain of A. muciniphila (MucT) delineated as SGB9226, was the most prevalent species in our cohort (>80% of Akkermansia positive subset) and was therefore used for the calculation of the relative abundance of Akk in the main figures of this article.

TABLE 2
Prevalence of various substrains of Akkermansia in our cohort.

| | SGB | | | | |
|---|---|---|---|---|---|
| | 9226~MucT - | 9228 - | 9224 - | 9223 - | None - |
| | no. (%) | no. (%) | no. (%) | no. (%) | no. (%) |
| Prevalence of SGBs in | 131/338 (38.8) | 26/338 (7.7) | 5/338 (1.5) | 1/338 (0.3) | 175/338 (51.7) |
| all patients (n = 338) | | | | | |
| Prevalence of SGBs in | 131/163 (80.4) | 26/163 (15.9) | 5/163 (3.1) | 1/163 (0.6) | — |
| SGBs⁺ patients (n = 163) | | | | | |

SGBs: Akkermansia candidate species or species-level genome bins; MucT: A. muciniphila.

We found msp_0025 to correspond to SGB9226, and used its relative abundance values as a proxy for A. muciniphila in MetaOMineR. A full description of both DNA purification and metagenomic pipelines is available in Derosa et al (Lisa Derosa et al. 2020). Starting from abundance matrices, only taxa that were present in at least 2.5% of all samples were considered, and then raw data were normalized and standardized (Sci-Kit-learn version 0.20.3).

### Tumor RNA Sequencing:

Using previously published technique (Fumet et al. 2018) total RNA was extracted from formalin-fixed paraffin-embedded (FFPE) tumors from patients with advanced NSCLC included in the main analysis as well as from patients with limited stages (Table 3). Libraries were prepared from 12 μl of total RNA with the TruSeq Stranded Total RNA using Ribo-Zero (Illumina) following manufacturer instructions. BBMAP v38.87 was used to trim the sequencing adapters and filtered the low quality and too short reads. Kallisto software (Bray et al. 2016) was used for quantifying transcript abundance from RNA-seq data against GRCh38 cDNA reference transcriptome from the Ensembl database, release 101. Only protein-coding transcripts and genes were included in the downstream analysis. Transcript Per Million values have been used for downstream analysis. Mann-Whitney tests have been performed to compare gene expression according to Akkermansia groups. PERMANOVA test with Euclidian distance has been used to assess the difference between groups on the subset of differentially expressed genes.

TABLE 3
Patient characteristics for tumor biopsies used in RNA sequencing.

| | | Overall | | Akk− | | Akk+ | | |
|---|---|---|---|---|---|---|---|---|
| | | (n = 44) | | (n = 22) | | (n = 22) | | P-value |
| Stage - no. (%) | Limited | 17 | (39) | 9 | (41) | 8 | (36) | 1.00 |
| | Advanced | 27 | (61) | 13 | (59) | 14 | (64) | |
| Age (year) | Median (range) | 69 | (55-82) | 68 | (58-78) | 73 | (55-82) | |
| | <65 yr | 12 | (27) | 6 | (28) | 6 | (28) | 0.55 |
| | ≥65 to <75 yr | 19 | (43) | 11 | (50) | 8 | (36) | |
| | ≥75 yr | 13 | (30) | 5 | (22) | 8 | (36) | |
| Sex - no. (%) | Male | 22 | (50) | 9 | (41) | 13 | (59) | 0.37 |
| | Female | 22 | (50) | 13 | (59) | 9 | (41) | |
| ECOG performance | 0-1 | 39 | (89) | 20 | (91) | 19 | (86) | 1.00 |
| status - no. (%) | | | | | | | | |
| | 2 or more | 5 | (11) | 2 | (9) | 3 | (14) | |
| Smoking status - no. (%) | Never smoked | 2 | (5) | 0 | (0) | 2 | (9) | 0.48 |
| | Current/former | 42 | (95) | 22 | (100) | 20 | (91) | |
| | smoker | | | | | | | |
| Tumor histology - no. (%) | Squamous | 12 | (27) | 6 | (27) | 6 | (28) | 1.00 |
| | Non-squamous | 28 | (64) | 14 | (64) | 14 | (63) | |
| | Others | 4 | (9) | 2 | (9) | 2 | (9) | |
| PD-L1 status - no. (%) | <1% | 6 | (14) | 1 | (5) | 5 | (23) | 0.60 |
| | 1-49% | 6 | (14) | 4 | (18) | 2 | (9) | |
| | ≥50% | 21 | (48) | 10 | (45) | 11 | (50) | |
| | Unknown | 11 | (24) | 7 | (32) | 4 | (18) | |

Statistical analyses: Chi-Square or Fisher's exact tests.
All tests were two-sided, with no adjustments for multiple comparisons.

### Pre-Clinical Study Details:

Mice. All animal experiments were carried out in compliance with French and European laws and regulations. The local institutional animal ethics board (Ministère de la Recherche, de l'Ènseignement Superieur er de l'Innovation) approved all mice experiments (permission numbers: 2016-049-4646, 2018-020-510263031v3). Mice avatar studies have been approved by the regulatory animal facility local and national committees (Ministère de la Recherche, de l'Enseignement Supérieur et de l'Innovation) (Everimmune #13366-2018020510263031 v3, APAFIS #17530-20181 1413352738 v2 (March 2019-March 2024). APAFIS #21378-201907080848483459). Female C571Bl/6 and BALE/c were purchased from Harlan (France) and Janvier (France), respectively. Mice were used between 8 and 16 weeks of age housed in specific pathogen-free conditions (SPF). All mouse experiments were performed at the animal facility in Gustave Roussy Cancer Campus where animals were housed in SPF conditions.

Cell culture, reagents and tumor cell lines. MC38, MCA-205 and B16F10 (syngeneic from C57BL/6 mice), and 4T1 cell lines (syngeneic from BALB/c mice) were purchased from ATCC. 4T1, MCA-205 and MC38 cells were cultured in RPMI 1640 containing 10% FCS, 2 mM L-glutamine, 100 UI/ml penicillin/streptomycin, 1 mM sodium pyruvate and MEM non-essential amino. All reagents were purchased from Gibco-Invitrogen (Carlsbad, CA, USA). B16F10 and CT26 cells were cultured in DMEM containing containing 10% FCS, +100 UI/ml penicillin/streptomycin+non-essential amino acid. All cell lines were cultured at 37° C. with 5% CO2 and regularly tested to be free of Mycoplasma contamination.

Subcutaneous model of MCA-205, MC38 and B16F10 and 4T1. Syngeneic C57BL/6 mice were respectively implanted with 0.8×10⁶ MCA-205, 1.0×10⁶ MC38/CT26 or 3×10⁵ B16F10 cells subcutaneously. Syngeneic BALB/c mice were implanted with 3×10⁵ 4T1 cells subcutaneously. For tumor growth experiments, tumor-implanted mice were treated intraperitoneally (i.p.) when tumors reached 20 to 40 mm² in size with anti-PD-1 mAbs (250 pg/mouse; clone RMP1-14, lot 695318A1) or isotype control (clone 2A3, lot 686318F1). Mice were injected 4 times at 3-day intervals with anti-PD-1 mAbs. Tumor length and width were routinely monitored every 3 times per week by means of a caliper. All antibodies were purchased from BioXcell, NH, US.

Antibiotic treatments. Mice were treated with an antibiotic solution (ATB) containing ampicillin (1 mg/ml), streptomycin (5 mg/ml), and colistin (1 mg/ml) (Sigma-Aldrich) added in the drinking water of mice. Antibiotic activity was confirmed by cultivating fecal pellets resuspended in BHI+15% glycerol at 0.1 g/ml on COS (Columbia Agar with 5% Sheep Blood) plates for 48 h at 37° C. in aerobic and anaerobic conditions. In brief, in the context of fecal microbial transplantation experiments, mice received 3 days of ATB before undergoing fecal microbial transplantation the next day by oral gavage using animal feeding needles.

FMT experiments. Fecal microbiota transfer (FMT) was performed by thawing fecal material. Two hundred μL of the suspension was then transferred by oral gavage into ATB pre-treated recipient (as described above). In addition, another 100 μL was applied on the fur of each animal. Two weeks after FMT, tumor cells were injected subcutaneously and mice were treated with anti-PD-1 mAbs or isotype control as previously explained. We used MCA-205 fibrosarcomas because it is normally-in SPF eubiotic mice-sensitive to anti-PD-1 Ab and has been used as a reference mouse model in our previous avatar experiments reported in (Routy, Le Chatelier, et al. 2018) and (Lisa Derosa et al. 2020), both papers showing that results obtained with MCA-205 were recapitulated in orthotopic TC1 lung cancer or RENCA models, respectively. So, we can trust the biological relevance and suitability of this MCA-205 model system to probe FMT or taxonomic fecal composition in future experiments.

Murine meta-analysis (FIG. 6E-F). We conducted 29 individual experiments over 2 years comprising 6-8 groups (including 6 mice/group) where the growth kinetics of orthotopic MCA-205 sarcomas (and other tumors such as MC38 colon cancer (syngeneic from C57BL/6 mice), or 4T1 breast or CT26 colon tumors (syngeneic from BALB/c mice) or B16 (melanoma) were monitored in avatar mouse models (Routy, Gopalakrishnan, et al. 2018). These recipients were ATB-treated and then received fecal microbial transplants (FMT) from 26 different NSCLC patients (phenotype of “Responder, R “versus” Non Responder, NR” to PD1 blockade, and relative abundance of stool Akk, both described in Table 4). Then, the mice were implanted with syngeneic tumors and later subjected to immunotherapy with anti-PD-1 antibodies. FMT could confer sensitivity (when stools were Akk⁺) or resistance (when stools were Akk−) to anti-PD-1 Ab, when compared with animals in eubiosis reared in SPF conditions (without FMT). Next, we analyzed the benefit and compensatory effects of oral supplementation of Akkermansia p2261, according to stool Akk⁺/⁻ in the original FMT. Of note, exogenous Akkermansia p2261 was not detectable more than 30 hrs in the recipient intestines (as shown in qRT-PCR using Akkermansia p2261 specific probe sets). To better scrutinize whether the exogenous Akkermansia p2261 could shift the microbiome of the recipient tumor bearers differently in R versus NR mice, we concatanated all tumor models syngeneic of BALB/c mice (CT26, 4T1) and C57BL/6 mice (E16F10, MCA-205-MC38) that were treated by FMT from 29 different NSCLC patients and then treated with anti-PD-1 mAbs+/−Akkermansia p2261 prior to stool harvesting for 16S rRNA based-sequencing of fecal amplicons. We calculated the relative benefit of exogenous Akk by dividing the ratio of tumor size between anti-PD-1+Akkermansia p2261/anti-PD-1+ water) normalized on the ratio of tumor sizes between anti-PD-1/Ctl mAbs in SPF mice for each time points for the whole kinetics. The non-supervised hierarchical clustering of the ratios between the tumor growth kinetics with anti-PD-1 mAbs with or without exogenous Akkermansia p2261 normalized on the effect of PD-1 blockade in eubiotic conditions revealed that exogenous Akkermansia p2261 was effective in about 50% cases (heatmap in the FIG. 6E). To delineate which Akkermansia p2261-associated ecosystem was associated with responses (ratios>mean of the cohort), LEfSe was used to identify the taxonomic changes segregating R versus NR to exogenous Akkermansia p2261. These species were compared with the bacteria featuring in human stools described in FIG. 4 and FIG. 3A.

TABLE 4A
Patient clinical characteristics

| | | Overall | Akk^{low} | Akk^{high} | Akk⁻ | |
|---|---|---|---|---|---|---|
| | | (n = 338) | (n = 101) | (n = 30) | (n = 207) | P-value |
| Age (year) | Median (range) | 65 (24-92) | 66 (45-85) | 67 (44-83) | 64 (24-92) | |
| | <65 yr | 163 (48) | 44 (44) | 11 (37) | 108 (52) | 0.15* |
| | ≥65 to <75 yr | 118 (35) | 46 (45) | 11 (37) | 61 (29) | |
| | ≥75 yr | 57 (17) | 11 (11) | 8 (26) | 38 (18) | |
| Sex - no (%) | Male | 226 (67) | 66 (65) | 20 (67) | 140 (68) | 0.93 |
| | Female | 112 (33) | 35 (35) | 10 (33) | 67 (32) | |
| BMI, kg/cm² - | <25 | 204 (60) | 56 (55) | 18 (60) | 130 (63) | 0.54 |
| no (%) | [25-30[ | 94 (28) | 33 (33) | 7 (23) | 54 (26) | |
| | ≥30 | 36 (11) | 11 (11) | 5 (17) | 20 (10) | |
| | Unknown | 4 (1) | 1 (1) | 0 | 3 (1) | |
| ECOG | 0-1 | 268 (79) | 86 (85) | 25 (84) | 157 (75) | 0.05 |
| performance | 2 or more | 43 (13) | 6 (6) | 4 (13) | 33 (16) | |
| status - | Unknown | 27 (8) | 9 (9) | 1 (3) | 17 (8) | |
| no (%) | | | | | | |
| Smoking | Never smoked | 26 (8) | 11 (11) | 1 (3) | 14 (7) | 0.28 |
| status - | Current/former | 309 (91) | 89 (88) | 29 (97) | 191 (92) | |
| no. (%) | smoker | | | | | |
| | Unknown | 3 (1) | 1 (1) | 0 (0) | 2 (1) | |
| Tumor | Squamous | 78 (23) | 22 (22) | 12 (40) | 44 (21) | 0.07 |
| histology - | Non-squamous | 260 (77) | 79 (88) | 18 (60) | 163 (79) | |
| no. (%) | | | | | | |
| Tumor | IIA-IIIB | 37 (11) | 14 (14) | 1 (3) | 22 (11) | 0.26 |
| staging - | IV | 294 (87) | 85 (84) | 29 (97) | 180 (87) | |
| no. (%) | Unknown | 7 (2) | 2 (2) | 0 | 5 (2) | |
| TMB - | ≥10 mutations/ | 3 (1) | 1 (1) | 0 | 2 (1) | 0.41 |
| no. (%) | megabase | | | | | |
| | <10 mutations/ | 13 (4) | 5 (5) | 4 (13) | 4 (2) | |
| | megabase | | | | | |
| | Unknown | 322 (95) | 95 (94) | 26 (87) | 201 (97) | |
| PD-L1 status - | <1% | 64 (19) | 21 (21) | 10 (33) | 33 (16) | 0.26 |
| no. (%) | 1-49% | 53 (16) | 18 (18) | 4 (13) | 33 (16) | |
| | ≥50% | 118 (35) | 34 (36) | 8 (27) | 76 (36) | |
| | Unknown | 102 (30) | 28 (27) | 8 (27) | 66 (32) | |
| Therapy | Neoadjuvant | 3 (1) | 2 (2) | 0 | 1 (1) | 0.13 |
| line - | First line | 92 (27) | 28 (28) | 3 (10) | 61 (29) | |
| no. (%) | ≥Second line | 243 (72) | 71 (72) | 27 (90) | 145 (70) | |
| Therapy - | Immunotherapy | 327 (97) | 99 (98) | 30 (100) | 198 (96) | 0.31 |
| no. (%) | Immunotherapy | 11 (3) | 2 (2) | 0 (0) | 9 (4) | |
| | and Chemotherapy | | | | | |
| Previous | Chemotherapy | 239 (71) | 77 (76) | 24 (80) | 143 (69) | 0.84 |
| therapy - | Tyrosine kinase | 11 (3) | 3 (3) | 1 (3) | 7 (3) | |
| no. (%) | inhibitors | | | | | |
| | Others | 20 (6) | 5 (5) | 0 (0) | 15 (7) | |
| | Unknown | 4 (1) | 1 (1) | 2 (7) | 1 (0) | |
| Antibiotics - | Yes | 69 (20) | 15 (15) | 10 (33) | 44 (21) | 0.07 |
| no. (%) | No | 269 (80) | 86 (85) | 20 (67) | 163 (79) | |

BMI, Body Mass Index;
TMB, Tumor Mutational Burden.
Statistical analyses: Mann-Whitney*, Chi-Square or Fisher's exact tests.
All tests were two-sided, with no adjustments for multiple comparisons.

TABLE 4B
List of antibiotics (ATB) 60 days prior to ICI

| | | Overall | Akk^{low} | Akk ^{high} | Akk − | |
|---|---|---|---|---|---|---|
| | | (n = 69) | (n = 15) | (n = 10) | (n = 44) | P-value |
| Duration | ≤7 days | 23 (33) | 6 (43) | 4 (36) | 13 (30) | 0.80 |
| of ATB - | >7 days | 19 (28) | 2 (14) | 4 (36) | 13 (30) | |
| no. (%) | Unknown | 27 (39) | 6 (43) | 3 (27) | 18 (41) | |
| Type of ATB - | β-lactams ± | 48 (70) | 9 (64) | 11 (100) | 28 (64) | 0.95 |
| no. (%) | inhibitors | | | | | |
| | (±combination | | | | | |
| | with other ATB) | | | | | |
| | Quinolones | 9 (13) | 1 (7) | 1 (9) | 7 (16) | |
| | (±combination | | | | | |
| | with other ATB) | | | | | |
| | Macrolides | 5 (7) | 0 | 0 | 5 (11) | |
| | Sulfonamides | 8 (12) | 1 (7) | 0 | 4 (9) | |
| | Tetracyclines | 1 (1) | 0 | 0 | 1 (2) | |
| | Aminoglycosides | 1 (1) | 0 | 0 | 1 (2) | |
| | Unknown | 4 (6) | 1 (7) | 0 | 3 (7) | |
| ECOG | 0-1 | 50 (72) | 13 (87) | 8 (80) | 29 (66) | 0.11 |
| performance | 2> | 13 (19) | 0 | 2 (20) | 11 (25) | |
| status - | Unknown | 6 (9) | 2 (13) | 0 | 4 (9) | |
| no. (%) | | | | | | |
| Number of | 1 | 39 (57) | 11 (79) | 3 (27) | 25 (57) | 0.45 |
| ATB courses - | 2 | 15 (22) | 3 (21) | 5 (45) | 7 (16) | |
| no. (%) | 3 | 6 (9) | 0 | 1 (9) | 5 (11) | |
| | 4 | 2 (3) | 0 | 2 (18) | 0 (0) | |
| | Unknown | 6 (9) | 0 | 0 | 7 (16) | |
| Indication - | Prophylactic ATB | 3 (4) | 0 | 0 | 3 (7) | |
| no. (%) | Therapeutic ATB | 52 (75) | 14 (93) | 3 (30) | 35 (79) | 0.49 |
| | Unknown | 14 (21) | 1 (7) | 7 (70) | 6 (14) | |

ATB, Antibiotics.
Statistical analyses: Chi-Square or Fisher's exact tests.
All tests were two-sided, with no adjustments for multiple comparisons.

Gut colonization with Akkermansia CSUR p2261. Akkermansia CSUR p2261 was provided by the Institut hospitalo-universitaire Méditerranée Infection, Marseille, France. Akkermansia p2261 was grown on 5% sheep blood enriched Columbia agar (COS) plates in an anaerobic atmosphere created using 3 anaerobic generators (BioMerieux) at 37° C. for at least 72 h. Identification of the bacterium was performed using a Matrix-Assisted Laser Desorption/Ionization Time of Flight (MALDI-TOF) mass spectrometer (Microflex LT analyser, Bruker Daltonics, Germany). Colonization of ATB pre-treated mice was performed by oral gavage with 100 μl of suspension containing 1×10⁸ bacteria obtained from a suspensions of 10⁹ CFU/mL using a fluorescence spectrophotometer (Eppendorf) at an optical density of 600 nm in PBS. Five bacterial gavages were performed for each mouse: the first 24 h before the first injection of anti-PD-1 mAbs and, subsequently, four times on the same day of ICI.

Mouse fecal DNA extraction and microbiota characterization. Feces were harvested in each mouse and group for metagenomics between 7 and 14 days after start of immunotherapy. Samples were stored at −80° C. until processing. Preparation and sequencing of mouse fecal samples was performed at IHU Méditerranée Infection, Marseille, France. Briefly, DNA was extracted using two protocols. The first protocol consisted of physical and chemical lysis, using glass powder and proteinase K respectively, then processing using the Macherey-Nagel DNA Tissue extraction kit (Duren, Germany). The second protocol was identical to the first protocol, with the addition of glycoprotein lysis and deglycosylation steps. The resulting DNA was sequenced, targeting the V3-V4 regions of the 16S rRNA gene. Raw FASTQ files were analyzed with Mothur pipeline v.1.39.5 for quality check and filtering (sequencing errors, chimerae) on a Workstation DELL T7910 (Round Rock, Texas, United States). Raw reads were filtered and clustered into Operational Taxonomic Units (OTUs), followed by elimination of low-populated OTUs (till 5 reads) and by de novo OTU picking at 97% pair-wise identity using standardized parameters and SILVA rDNA Database v.1.19 for alignment. A prevalence threshold of 2.5% was implemented for statistical analyses on recognized OTUs, performed with Python v3.8.2. The most representative and abundant read within each OTU (as evidenced in the previous step with Mothur v.1.39.5) underwent a nucleotide Blast using the National Center for Biotechnology Information (NCBI) Blast software (ncbi-blast-2.9.0) and the latest NCBI 16S Microbial Database (ftp://ftp.ncbi.nlm.nih.gov/blast/db/). A matrix of bacterial relative abundances was built at each taxon level (phylum, class, order, family, genus, species) for subsequent multivariate statistical analyses.

### Statistical Analyses

In humans, data matrices were firstly normalized then standardized using QuantileTransformer and StandardScaler methods from Sci-Kit learn package v0.20.3. Normalization using the output_distribution=‘normal’ option transforms each variable to a strictly Gaussian-shaped distribution, whilst the standardization results in each normalized variable having a mean of zero and variance of one. These two steps of normalization followed by standardization ensure the proper comparison of variables with different dynamic ranges, such as bacterial relative abundances. For microbiota analysis, measurements of α diversity (within sample diversity) such as Richness and Shannon index, were calculated at species level using the SciKit-learn package v.0.4.1. Exploratory analysis of β-diversity (between sample diversity) was calculated using the Bray-Curtis measure of dissimilarity and represented in Principal Coordinate Analyses (PCoA), along with methods to compare groups of multivariate sample units (analysis of similarities—ANOSIM, permutational multivariate analysis of variance—PERMANOVA) to assess significance in data points clustering. ANOSIM and PERMANOVA were automatically calculated after 999 permutations, as implemented in SciKit-learn package v0.4.1. We implemented Partial Least Square Discriminant Analysis (PLS-DA) and the subsequent Variable Importance Plot (VIP) as a supervised analysis wherein the VIP values (order of magnitude) are used to identify the most discriminant bacterial species. All the analyses were performed within a Python v3.8.2 environment. Univariate differential abundance analysis was performed via linear discriminant analysis of effect size (LEfSe)(Segata et al. 2011). We added further support of differentially abundant species using two different multivariate differential abundance methods; ANCOM-BC (Lin et Peddada 2020) and MaAsLin2 (Mallick et al. 2020), that included covariates such as age, sex, BMI, cohort and sequencing batch. France's Data Protection Article 8 legislation (Commission Nationale Informatique et Libertés [CNIL]) prohibits the analysis of the racial and ethnic origins. Raw sequencing counts were estimated from species-level MetaPhlAn 3 relative abundances by multiplying these values by the total number of reads for each sample and these were used in ANCOM-BC (v.1.0.1) with default parameters, a library size cutoff of 500 reads and no structural zero detection. Masslin2 (v.1.4.0) was run using Logit transformed relative abundances that were normalized with total-sum-scaling (TSS) and using the variable of interest as a fixed effect.

Survival curves were estimated using the Kaplan-Meier method and compared with the log-rank test (Mantel-Cox method) in a univariate analysis. Multivariate analyses were performed using Cox regression models to determine HRs and 95% confidence intervals (Cis) for OS adjusting for other clinicopathologic features. The proportionality hazard assumption was checked testing the trend of the Schoenfeld residuals with the cox.zph R function. When the test was statistically significant for a variable, its interaction with time was introduced in the model and tested using the tt (time transformation) function with different functional forms (linear, exponential, logarithmic, and penalized spline). The optimal cutoff for each bacterial species to define different prognosis groups was obtained with grid search algorithm based on the multivariate Cox model to take into account the potential confounding factors (age, sexe, . . . ). The grid was defined for each species by the percentiles of the distribution of the non-zero prevalence values. The cutoff corresponding to the model with the better Akaike information criterion (AIC, lower is better) was selected as the optimal cutoff.

All tests were two-sided and statistical significance was set at a p-value<0.05. Statistical analyses were conducted using the GraphPad Prism 7 and R software (http://www.R-project.org/).

In mice, all tumor growth curves were analyzed using software developed in Kroemer's laboratory: https://kroemerlab.shinyapps.io/TumGrowth/. Between-group comparisons of mice, global comparison were performed using Kruskall-Wallis test, post-hoc multiple comparisons using Dunn's test. Finally, natural tumor growth data deriving from mice experiments (6 mice per experiment) were averaged for each timepoint (T0 to T8), then longitudinally normalized on the first timepoint, in order to have a common starting value of 1. All averaged and normalized tumor values were then expressed with Fold Ratios (FR, FIG. 9E), and underwent base 2 logaritmization in order to enhance augmentation (star on grey color code (the darker the greater) or diminution (grey color code (the darker the greater) of the tumor growth. A Hierarchical Clusterization Analysis (HCA) based on Bray-Curtis distance was implemented on longitudinal FR data in order to define a branch of responder (R) to Akkermansia p2261 (light grey cluster), or a group of non-responder (NR) to Akkermansia p2261 (dark grey cluster). All reported p-values underwent Benjamini-Hochberg two-stages False Discovery Rate (FDR) at 10%.

### Results

Association Between Akkermansia muciniphila and Clinical Outcome

From December 2015 to November 2019, a total of 493 patients were screened for enrollment in this study and 338 patients met inclusion criteria, providing at least one baseline (V1 and/or V2) fecal sample for profiling (FIG. 4). Using shotgun metagenomic sequencing, we determined that Akk was detectable in 131 (39%) and absent in 207 (61%) patients using MetaPhlAn profiling expanded to identify the main Akk species-level genome bin (SGB9226 spp (Karcher et al. 2021), Table 5). Baseline characteristics were well balanced between patients with detectable Akk (Akk⁺) and undetectable Akk (Akk⁻) groups with respect to sex, ECOG performance status, smoking history, tumor histology, body mass index (BMI), PD-L1 expression, and line of therapy (Table 5A). However, there was a non-statistically significant trend for older age in the Akk⁺ group (66 years versus 64 years, p=0.08). A total of 69 patients (20%) received antibiotics (ATB) 60 days prior to ICI initiation, with similar frequencies (p=0.68) and regimen in Akk⁺ and Akk⁻ groups (Table 5A). Among ATB classes, beta-lactams were the most commonly prescribed in both groups (Table 5B, 80% in Akk⁺ versus 64% in Akk⁻, p=0.33).

TABLE 5A
Patient clinical characteristics

| | | Overall | | Akk⁺ | | Akk⁻ | | |
|---|---|---|---|---|---|---|---|---|
| | | (n = 338) | | (n = 131) | | (n = 207) | | P-value |
| Age (year) | Median (range) | 65 | (24-92) | 66 | (44-85) | 64 | (24-92) | 0.08* |
| | <65 yr | 163 | (48) | 55 | (42) | 108 | (52) | |
| | ≥65 to <75 yr | 118 | (35) | 57 | (43) | 61 | (29) | |
| | ≥75 yr | 57 | (17) | 19 | (15) | 38 | (18) | |
| Sex - no (%) | Male | 226 | (67) | 86 | (66) | 140 | (68) | 0.72 |
| | Female | 112 | (33) | 45 | (34) | 67 | (32) | |
| BMI, kg/cm² - no | <25 | 204 | (60) | 74 | (56) | 130 | (63) | 0.66 |
| (%) | [25-30[ | 94 | (28) | 40 | (31) | 54 | (26) | |
| | ≥30 | 36 | (11) | 16 | (12) | 20 | (10) | |
| | Unknown | 4 | (1) | 1 | (1) | 3 | (1) | |
| ECOG performance | 0-1 | 268 | (79) | 111 | (85) | 157 | (76) | 0.08 |
| status - no (%) | 2 or more | 43 | (13) | 10 | (8) | 33 | (16) | |
| | Unknown | 27 | (8) | 10 | (8) | 17 | (8) | |
| Smoking status - | Never smoked | 26 | (8) | 12 | (9) | 14 | (7) | 0.73 |
| no. (%) | Current/former | 309 | (91) | 118 | (90) | 191 | (92) | |
| | smoker | | | | | | | |
| | Unknown | 3 | (1) | 1 | (1) | 2 | (1) | |
| Tumor histology - | Squamous | 78 | (23) | 34 | (26) | 44 | (21) | 0.35 |
| no. (%) | Non-squamous | 260 | (77) | 97 | (74) | 163 | (79) | |
| Tumor staging - | IIA-IIIB | 37 | (11) | 15 | (11) | 22 | (11) | 0.97 |
| no. (%) | IV | 294 | (87) | 114 | (87) | 180 | (87) | |
| | Unknown | 7 | (2) | 2 | (2) | 5 | (2) | |
| TMB - no. (%) | ≥10 mutations/ | 3 | (1) | 1 | (1) | 2 | (1) | 0.25 |
| | megabase | | | | | | | |
| | <10 mutations/ | 13 | (4) | 9 | (7) | 4 | (2) | |
| | megabase | | | | | | | |
| | Unknown | 322 | (95) | 121 | (92) | 201 | (97) | |
| PD-L1 status - no. | <1% | 64 | (19) | 31 | (24) | 33 | (16) | 0.41 |
| (%) | 1-49% | 53 | (16) | 22 | (17) | 33 | (16) | |
| | ≥50% | 118 | (35) | 42 | (32) | 76 | (36) | |
| | Unknown | 102 | (30) | 36 | (27) | 66 | (32) | |
| Therapy line - no. | Neoadjuvant | 3 | (1) | 2 | (2) | 1 | (1) | 0.33 |
| (%) | First line | 92 | (27) | 31 | (24) | 61 | (29) | |
| | ≥Second line | 243 | (72) | 98 | (75) | 145 | (70) | |
| Therapy - no. (%) | Immunotherapy | 327 | (97) | 129 | (98) | 198 | (96) | 0.21 |
| | Immunotherapy | 11 | (3) | 2 | (2) | 9 | (4) | |
| | and Chemotherapy | | | | | | | |
| Previous therapy - | Chemotherapy | 239 | (71) | 96 | (73) | 143 | (69) | 0.77 |
| no. (%) | Tyrosine kinase | 11 | (3) | 4 | (3) | 7 | (3) | |
| | inhibitors | | | | | | | |
| | Others | 20 | (6) | 6 | (5) | 15 | (7) | |
| | Unknown | 3 | (1) | 2 | (2) | 1 | (0) | |
| Antibiotics - no. | Yes | 69 | (20) | 25 | (19) | 44 | (21) | 0.68 |
| (%) | No | 269 | (80) | 106 | (81) | 163 | (79) | |

BMI, Body Mass Index; TMB, Tumor Mutational Burden.
Statistical analyses: Mann-Whitney*, Chi-Square or Fisher's exact tests are two-sided, with no adjustments for multiple comparisons.

TABLE 5B
List of antibiotics (ATB) 60 days prior to ICB

| | | Overall | | Akk⁺ | | Akk⁻ | | |
|---|---|---|---|---|---|---|---|---|
| | | (n = 69) | | (n = 25) | | (n = 44) | | P-value |
| Duration of ATB - | ≤7 days | 23 | (33) | 10 | (40) | 13 | (30) | 0.73 |
| no. (%) | >7 days | 19 | (28) | 6 | (24) | 13 | (30) | |
| | Unknown | 27 | (39) | 9 | (36) | 18 | (41) | |
| Type of ATB - no. | β-lactams ± inhibitors (± | 48 | (70) | 20 | (80) | 28 | (64) | 0.33 |
| (%) | combination with other | | | | | | | |
| | ATB) | | | | | | | |
| | Quinolones (± | 9 | (13) | 2 | (8) | 7 | (16) | |
| | combination with other | | | | | | | |
| | ATB) | | | | | | | |
| | Macrolides | 5 | (7) | 0 | (0) | 5 | (11) | |
| | Sulfonamides | 8 | (12) | 4 | (16) | 4 | (9) | |
| | Tetracyclines | 1 | (1) | 0 | (0) | 1 | (2) | |
| | Aminoglycosides | 1 | (1) | 0 | (0) | 1 | (2) | |
| | Unknown | 4 | (6) | 1 | (4) | 3 | (7) | |
| ECOG performance | 0-1 | 50 | (72) | 21 | (84) | 29 | (66) | 0.08 |
| status - no. (%) | 2> | 13 | (19) | 2 | (8) | 11 | (25) | |
| | Unknown | 6 | (9) | 2 | (8) | 4 | (9) | |
| Number of ATB | 1 | 39 | (57) | 14 | (56) | 25 | (57) | 0.38 |
| courses - no. (%) | 2 | 15 | (22) | 8 | (32) | 7 | (16) | |
| | 3 | 6 | (9) | 1 | (4) | 5 | (11) | |
| | 4 | 2 | (3) | 2 | (8) | 0 | (0) | |
| | Unknown | 6 | (9) | 0 | (0) | 7 | (16) | |
| Indication - no. | Prophylactic ATB | 3 | (4) | 0 | | 3 | (7) | 0.23 |
| (%) | Therapeutic ATB | 52 | (75) | 17 | (68) | 35 | (79) | |
| | Unknown | 14 | (21) | 8 | (32) | 6 | (14) | |

ATB, Antibiotics.
Statistical analyses: Chi-Square or Fisher's exact tests.
All tests were two-sided, with no adjustments for multiple comparisons.

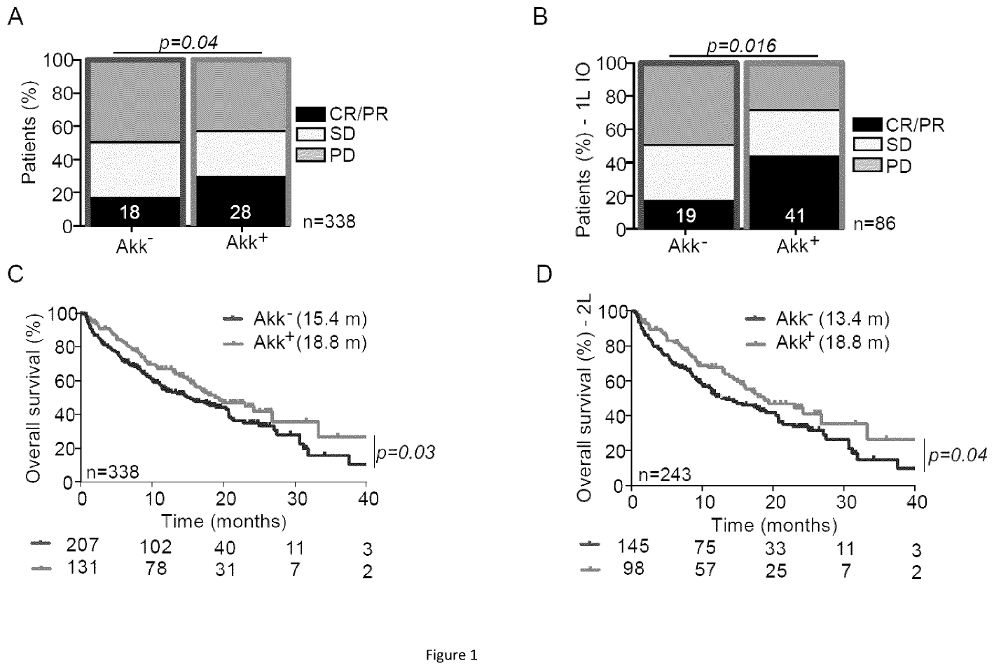

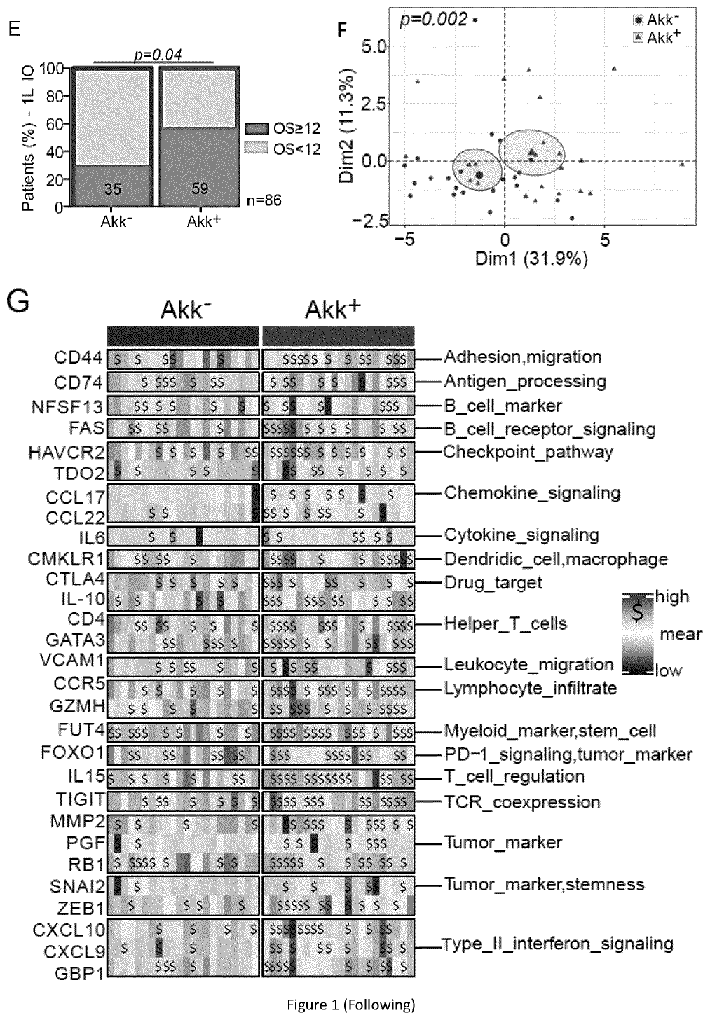

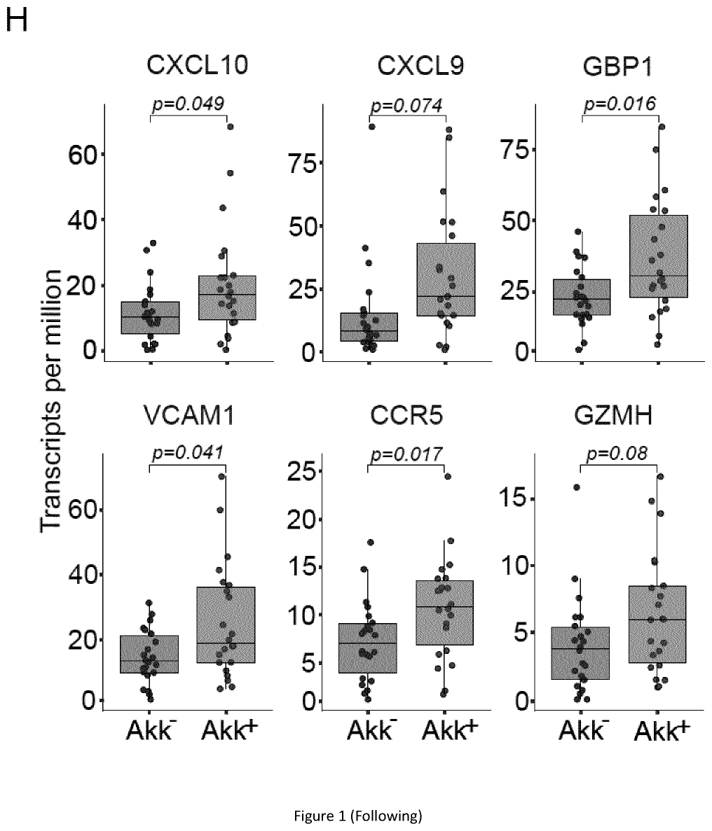

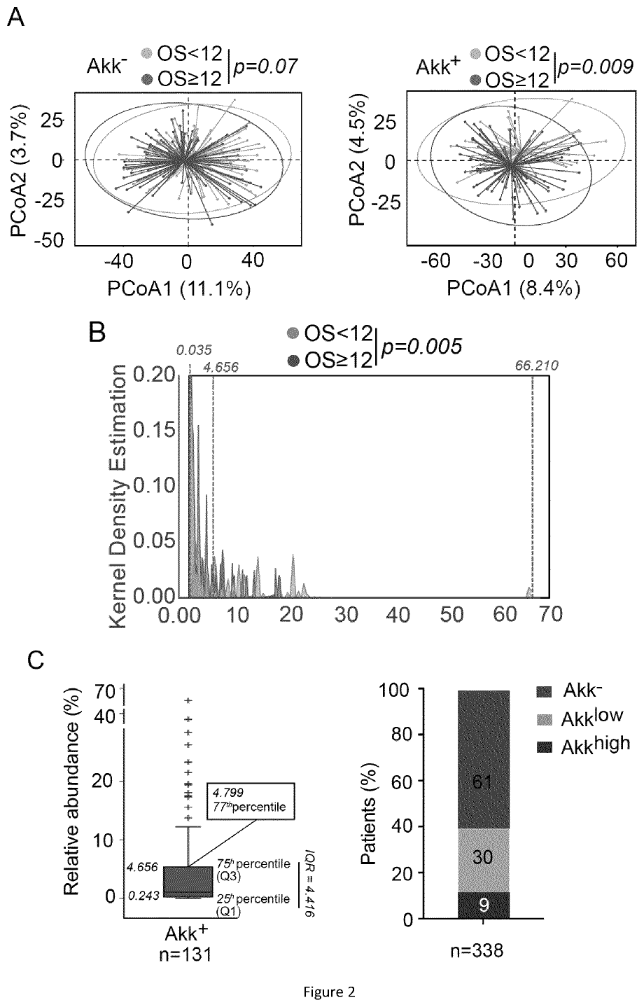

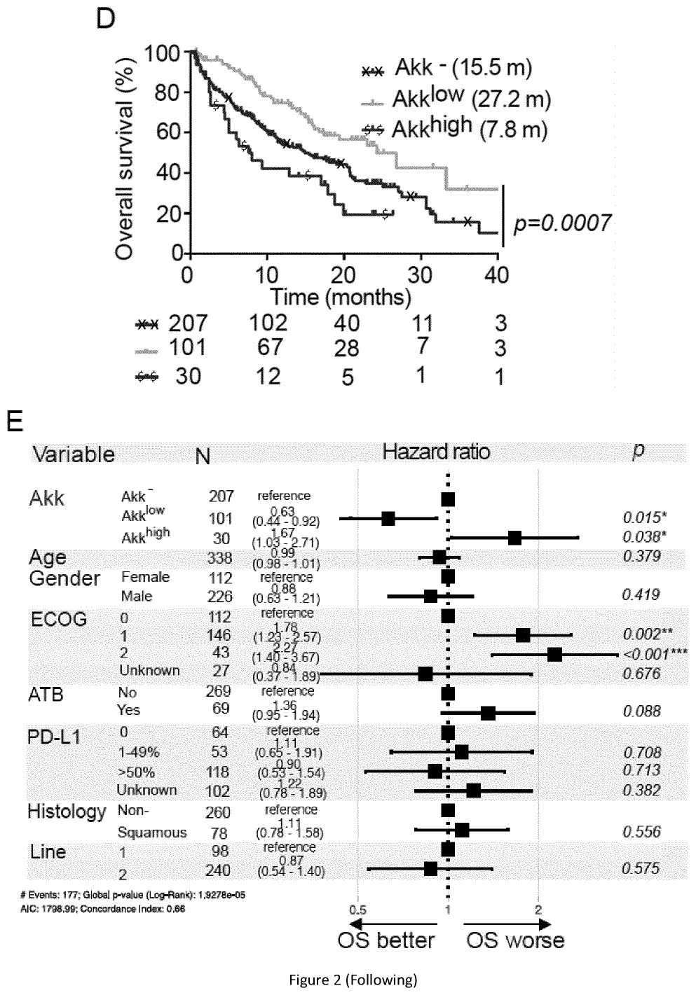

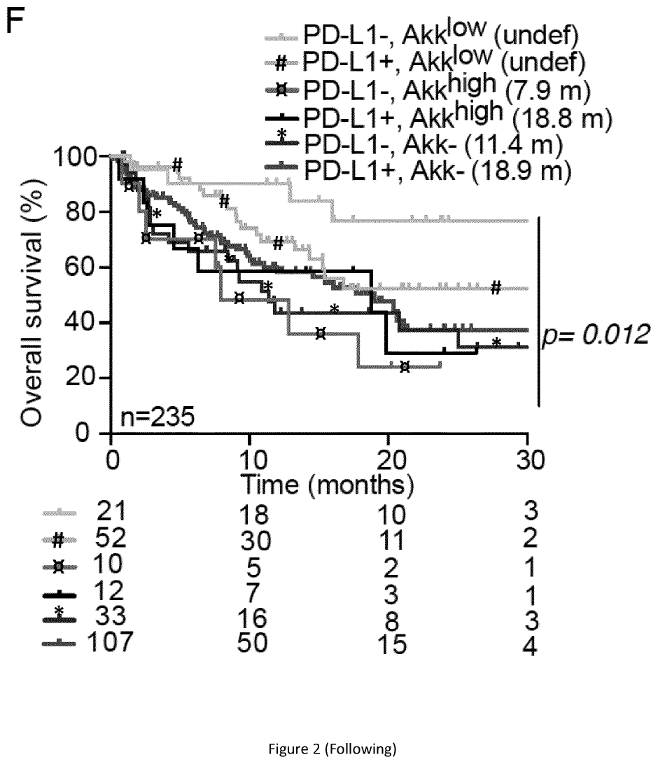

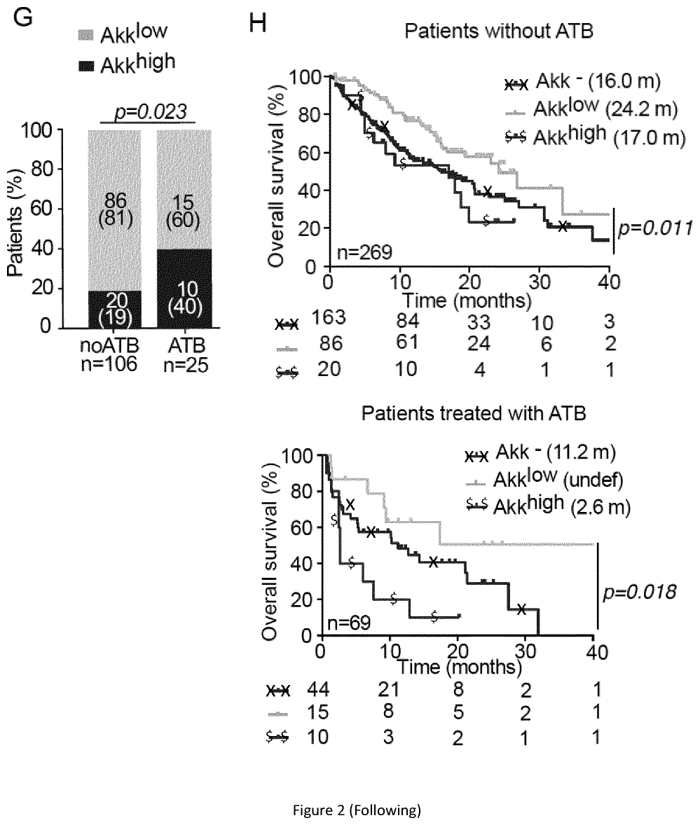

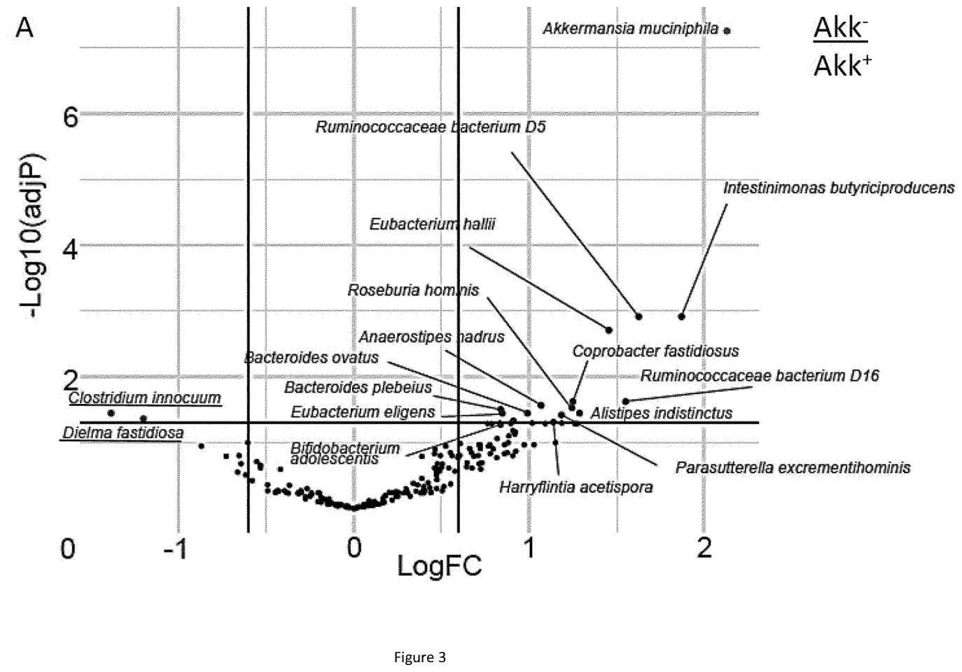

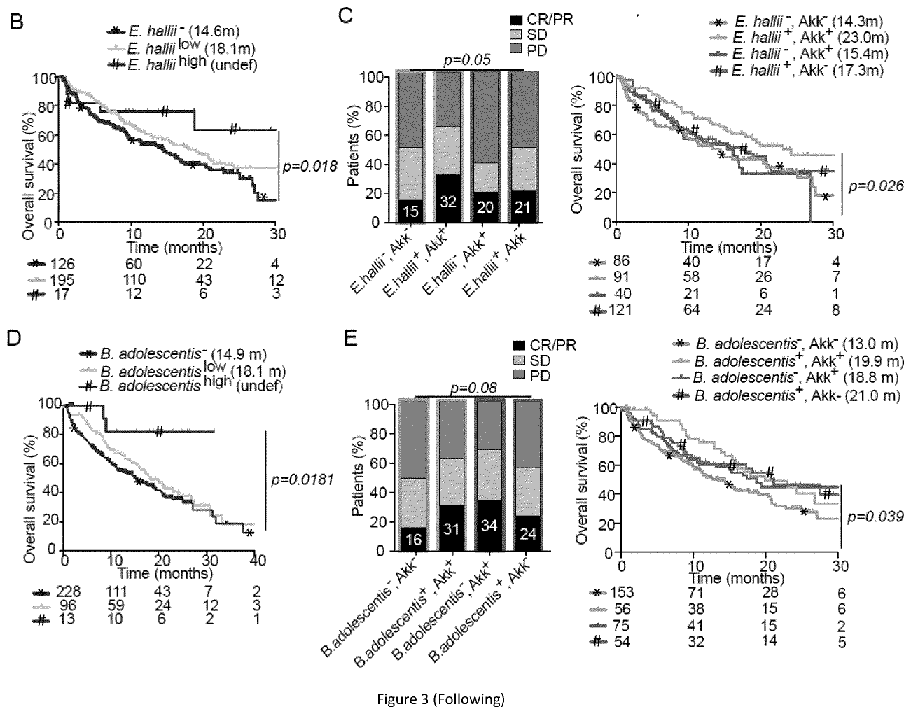

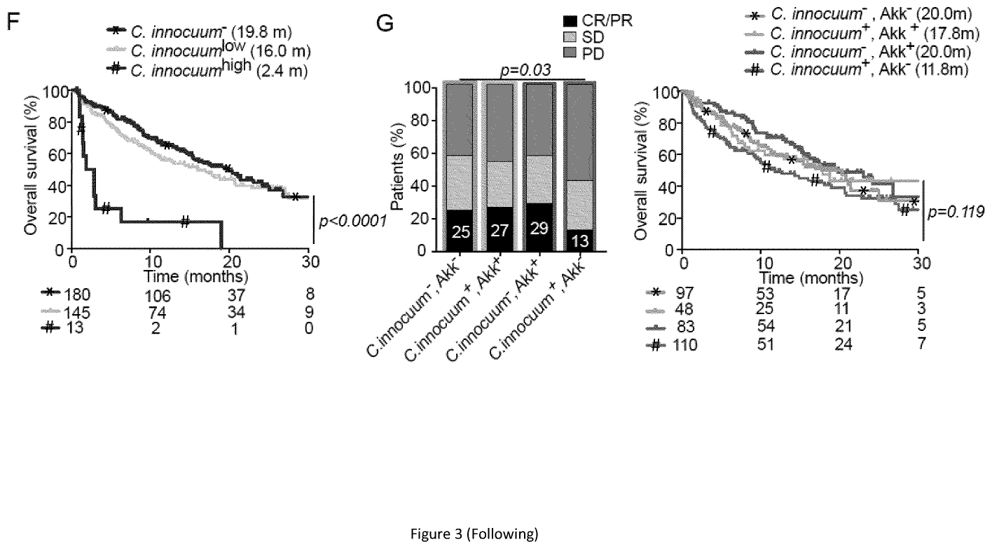

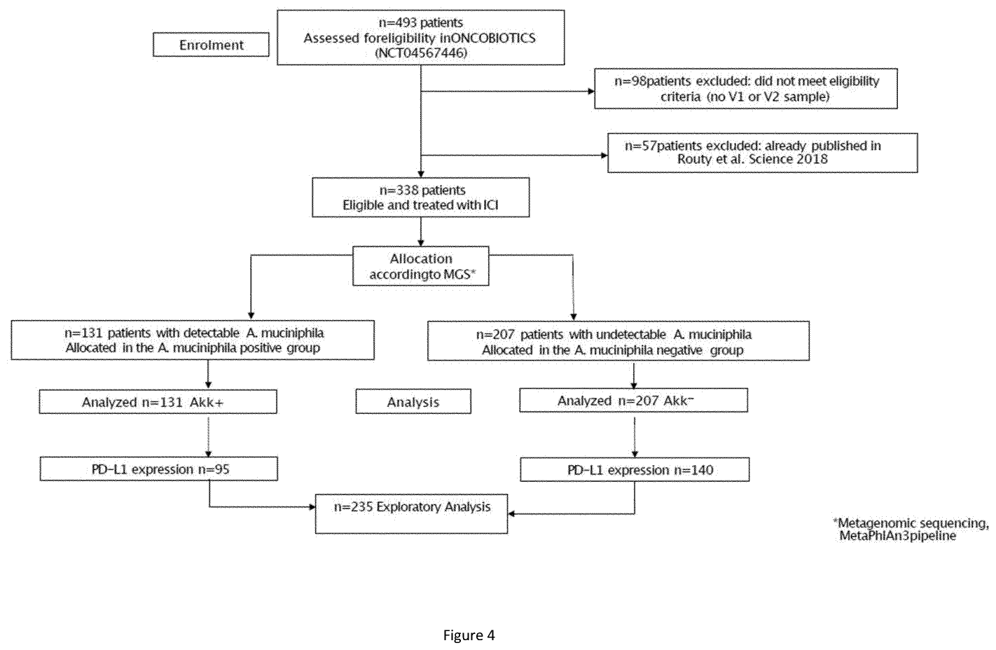

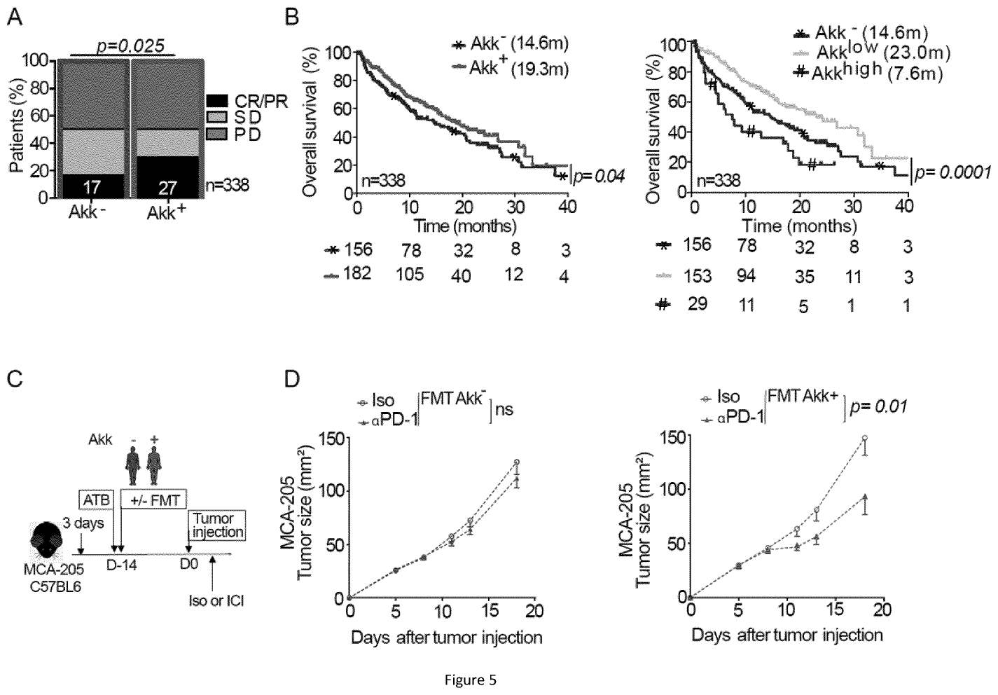

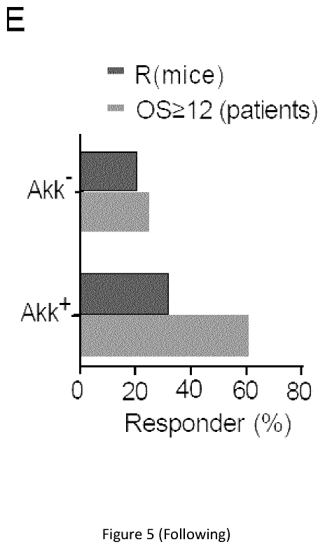

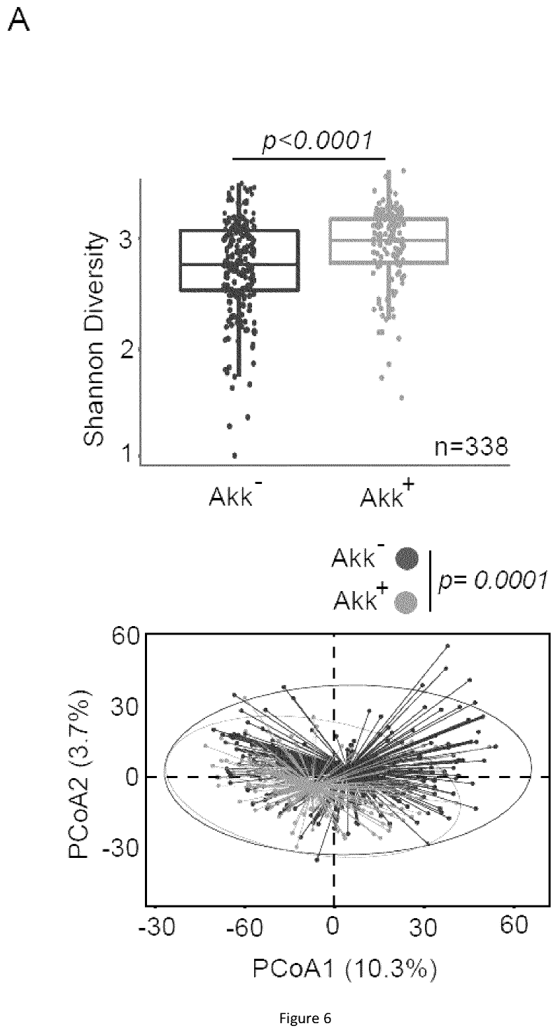

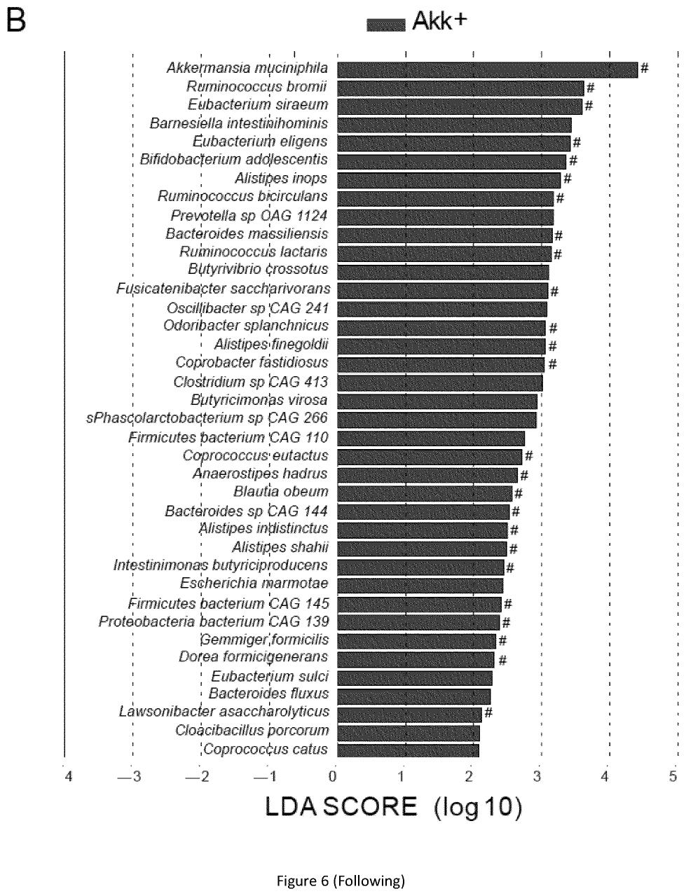

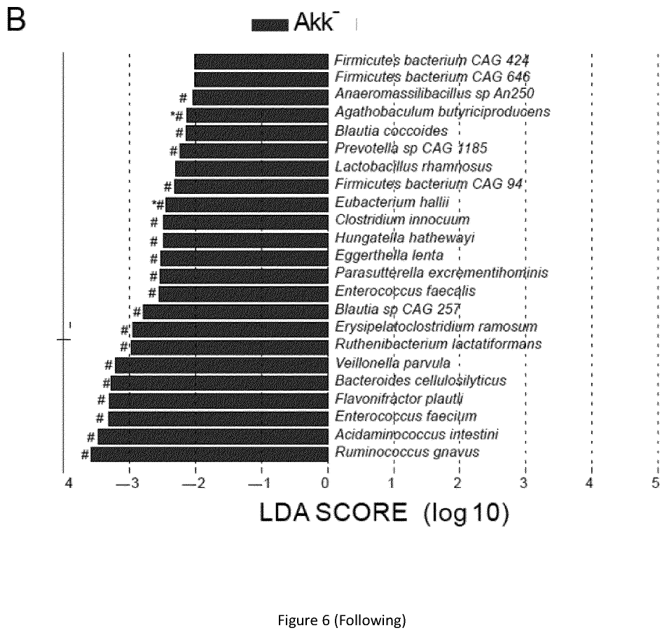

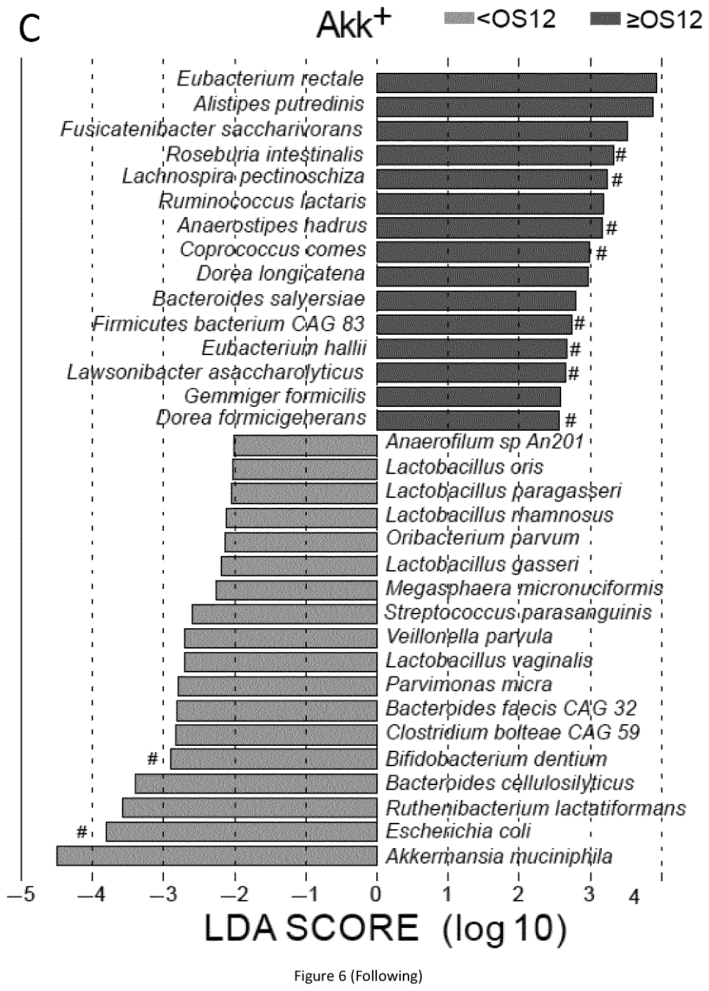

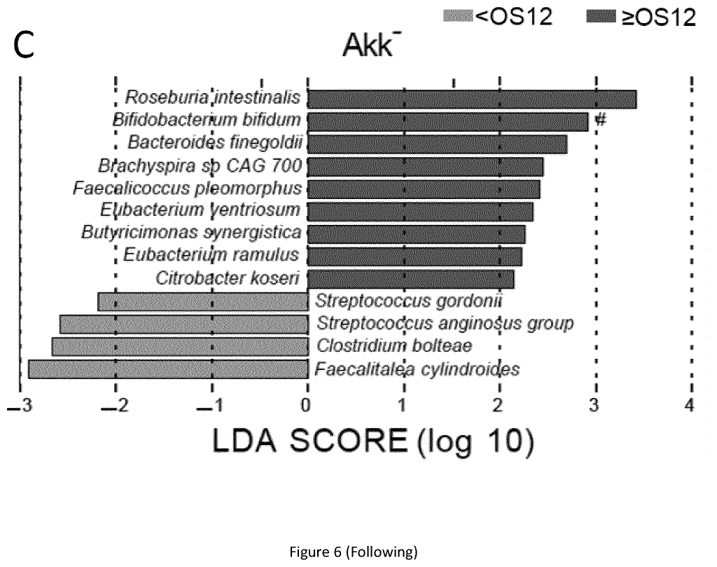

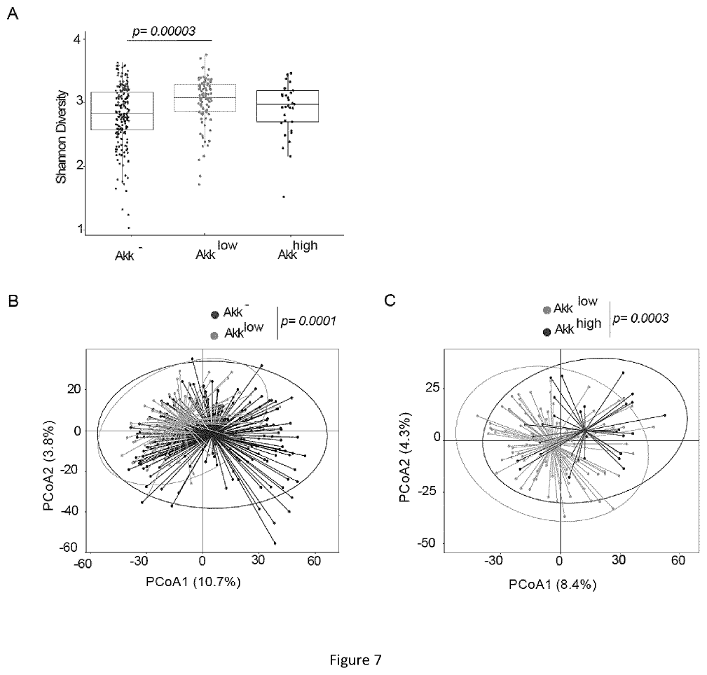

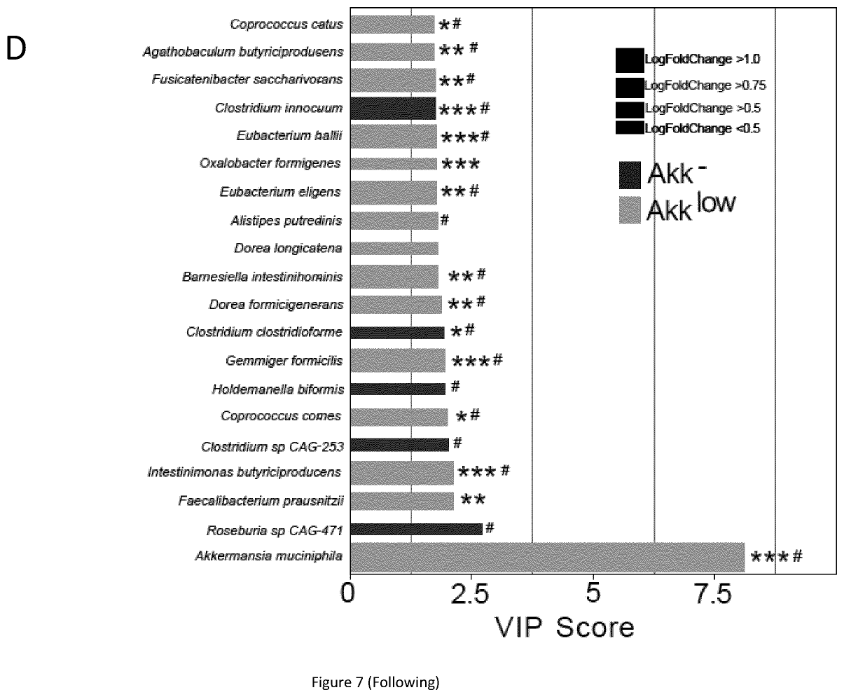

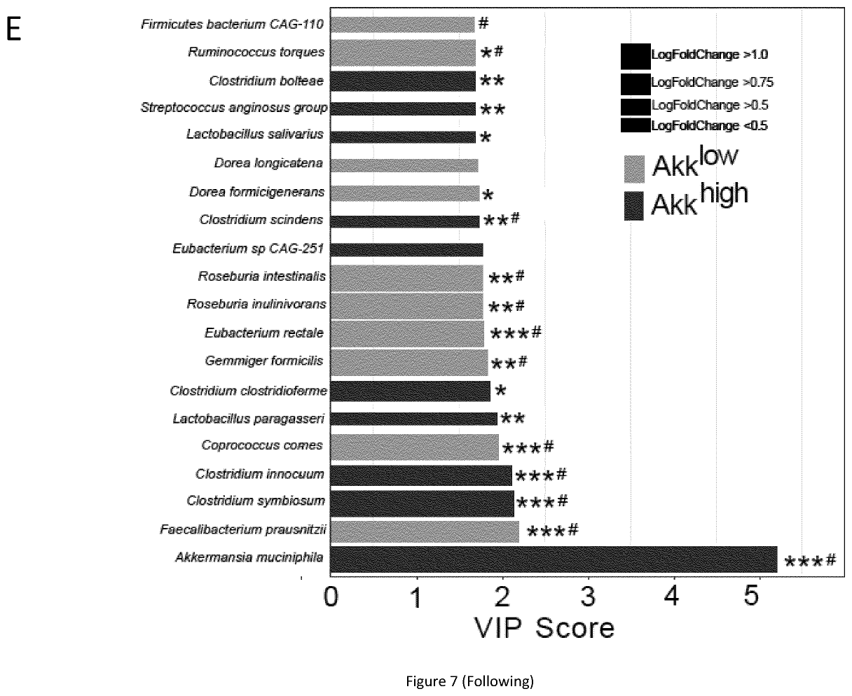

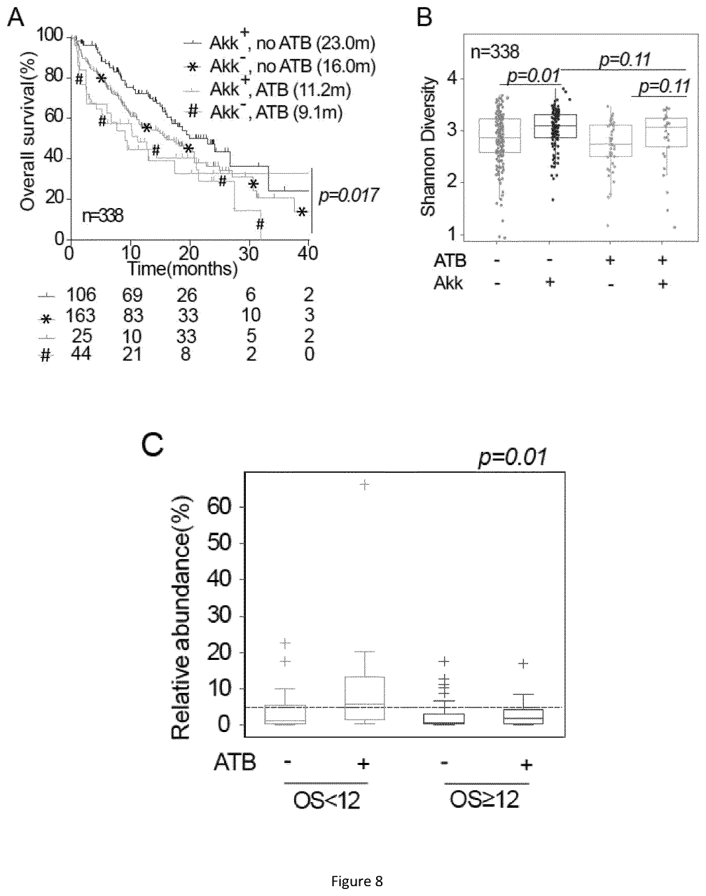

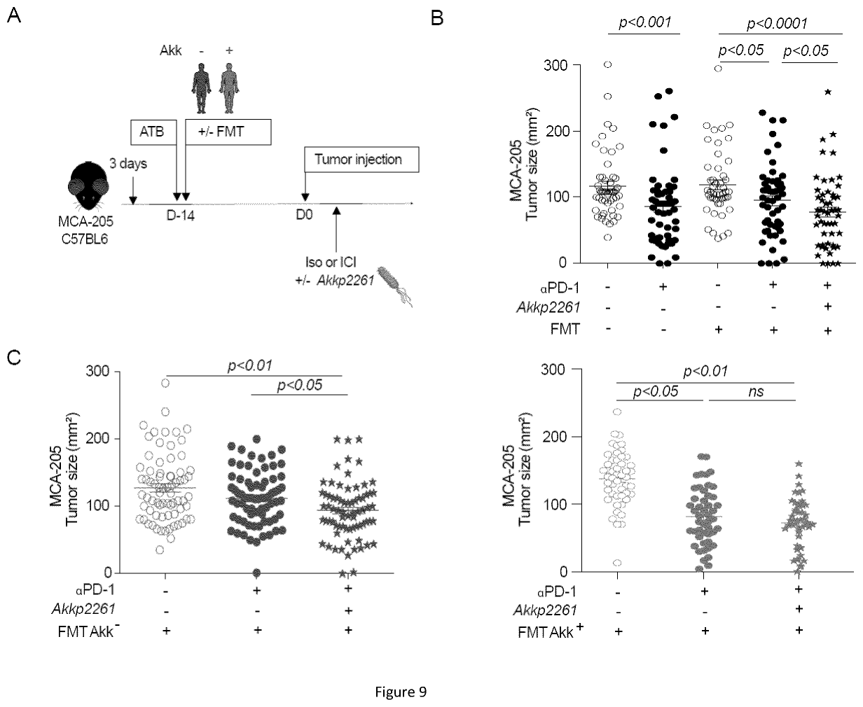

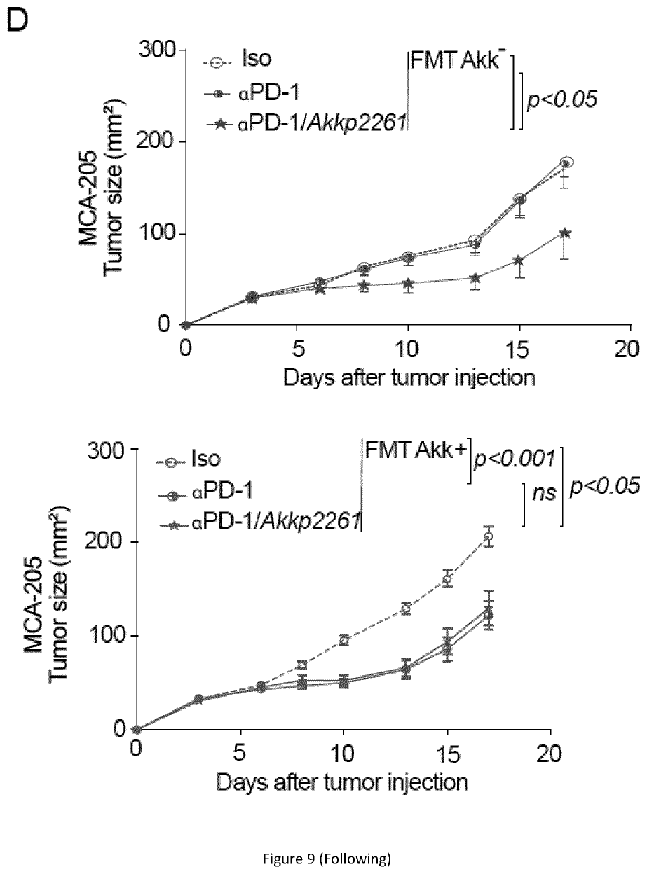

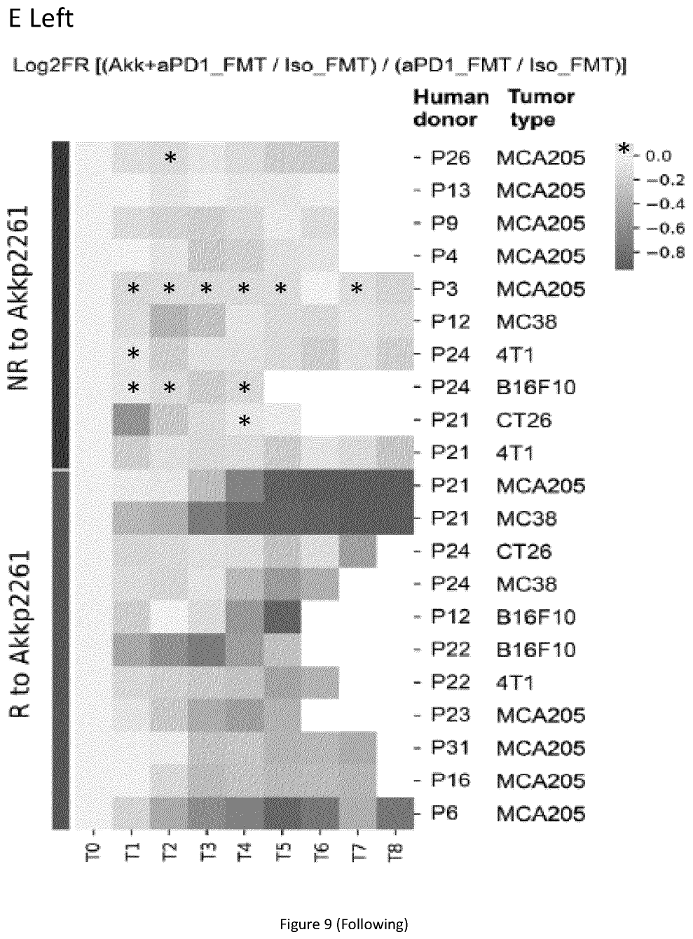

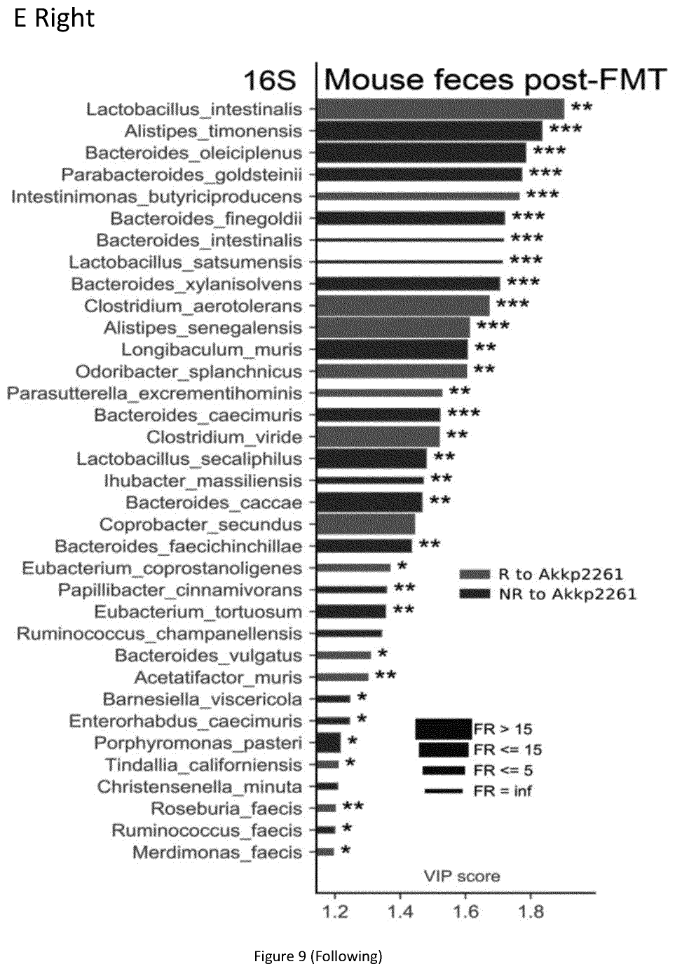

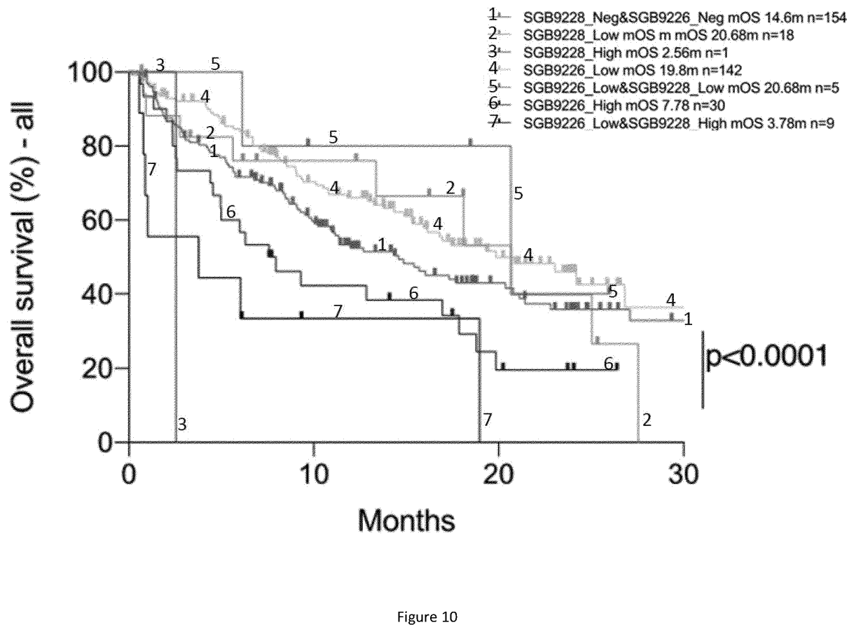

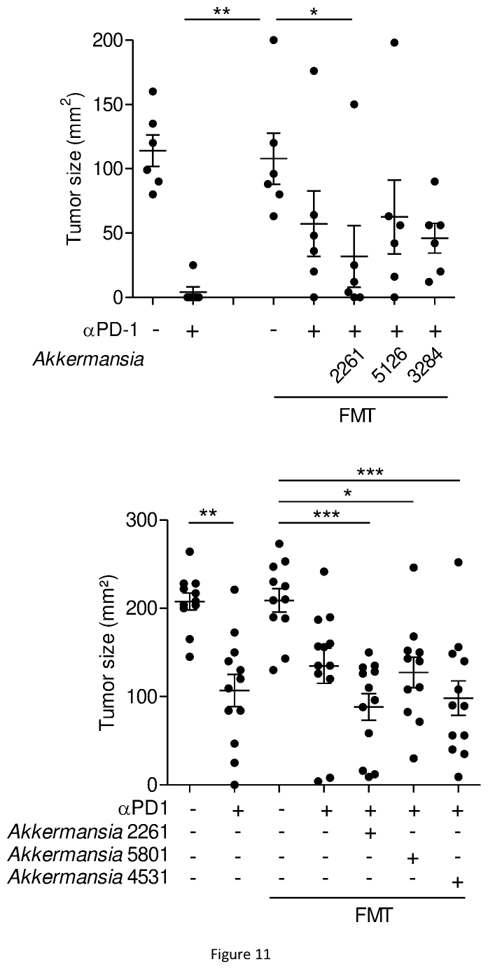

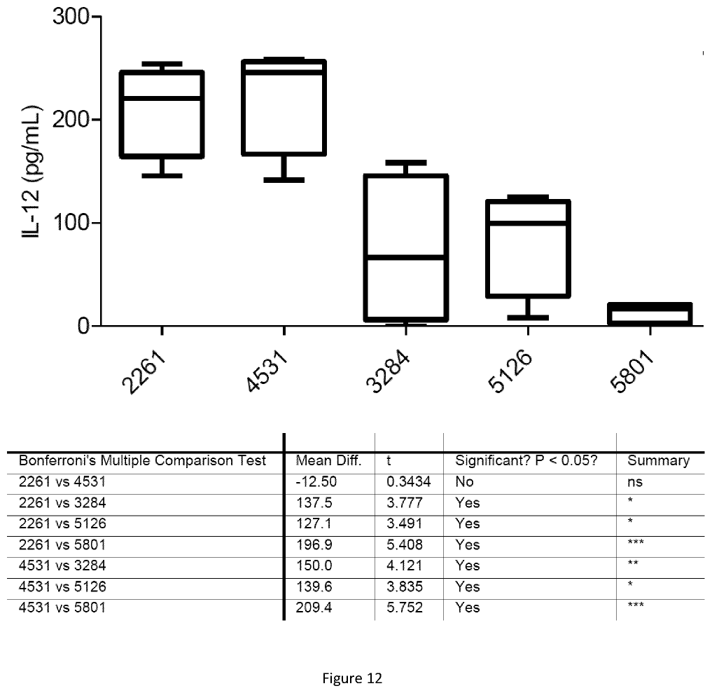

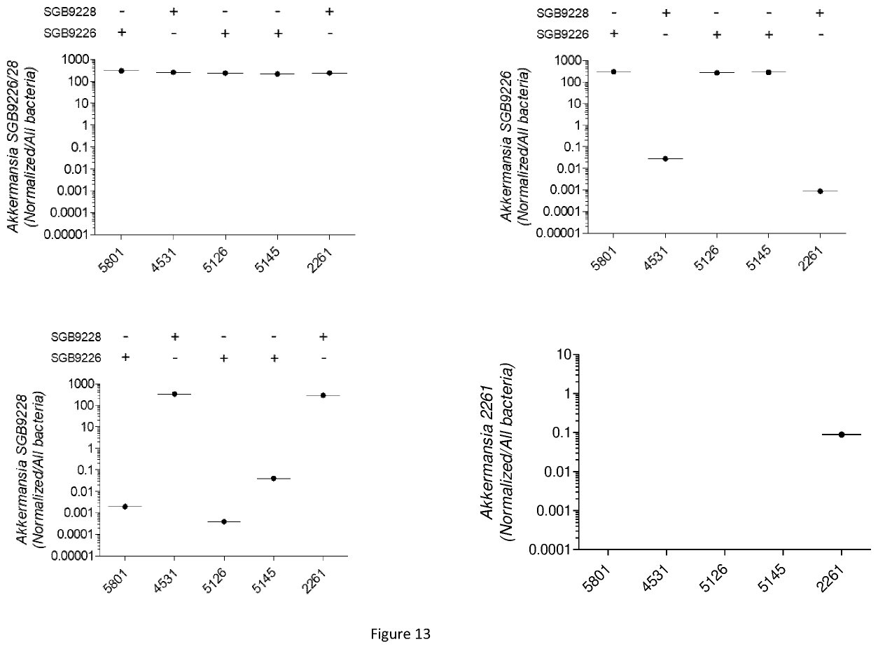

When considering Akk⁺ versus Akk⁻ groups in our cohort, objective response rates (ORR) were 28% and 18% respectively (FIG. 1A, p=0.04). Partial responses (PR), stable disease (SD) and progressive disease (PD) rates were 28%, 28% and 44%, respectively, for the Akk⁺ group versus 18%, 31% and 50% for the Akk⁻ group. When considering the subgroup of patients who received immunotherapy alone as front-line treatment (1L IO, n=86), ORR were 41% and 19% in the Akk⁺ versus Akk⁻ respectively (FIG. 1B, p=0.016). Of note, for the whole cohort of patients (irrespective of the line of treatment), the median overall survival (mOS) was 18.8 months for the Akk⁺ group compared to 15.4 months in the Akk⁻ group (HR=0.72; (95% CI:0.73-1.62); p=0.03, FIG. 1C, Table 5A). The median OS of ≥2L NSCLC patients was 18.8 months for the Akk⁺ group compared to 13.4 months in the Akk group (HR=0.70; (95% CI:0.50-0.98); p=0.04, FIG. 1D, Table 5A). For the 1 L IO patients, 59% of Akk⁺ patients were still alive after 12 months (OS≥12) while only 35% of Akk⁻ individuals were long term survivors (FIG. 1E, p=0.04). We draw the same conclusions when using the MetaOMineR pipeline (Le Chatelier et al. 2013) (FIG. 5A-B left panel).

To establish cause-effect relationships between the presence of Akk (and its ecosystem) with response to ICI, we retrospectively performed a preclinical meta-analysis gathering 29 experiments where the tumor growth kinetics were followed up in avatar mouse models (Routy, Gopalakrishnan, et al. 2018). These recipients were ATB-treated and then received fecal microbial transplants (FMT) from 26 different NSCLC patients (Table 6). Then, mice were implanted with syngeneic orthotopic MCA-205 sarcomas (representative tumor model for sensitivity to anti-PD-1 antibodies as previously described (Routy, Le Chatelier, et al. 2018; Lisa Derosa et al. 2020)) and later subjected to PD-1 blockade (FIG. 5C-E). As observed in our clinical cohort, resistance to therapy in mice was associated with the absence of detectable Akk in the FMT experiments (FIG. 5C-0).

Therefore, in this second independent and prospective study on 338 advanced NSCLC patients, we validated in humans using two different metagenomics pipelines as well as in mice that the presence of Akk is associated with higher ORR and longer OS in patients with NSCLC receiving ICI.

TABLE 6
Patient characteristics for mouse tumor models

| Human donors | | | | | Mice |
|---|---|---|---|---|---|
| ID | Type of cancer | A. muciniphila SGB 9226 | ORR | OS12 | Type of cancer |
| P1 | Lung | 0 | NR | <12 m | MCA205 |
| P2 | Lung | 4.14373 | NR | ≥12 m | MCA205 |
| P3 | Lung | 5.29109 | R | <12 m | MCA205 |
| P4 | Lung | 0 | NR | <12 m | MCA205 |
| P5 | Lung | 0 | NR | ≥12 m | MCA205 |
| P6 | Lung | 0 | NR | ≥12 m | MCA205 |
| P8 | Lung | 0 | NR | <12 m | MCA205 |
| P9 | Lung | 0 | NR | <12 m | MCA205 |
| P10 | Lung | 3.09375 | NR | ≥12 m | MCA205 |
| P11 | Lung | 0 | NR | <12 m | MCA205 |
| P13 | Lung | 0 | NR | <12 m | MCA205 |
| P14 | Lung | 0 | NR | <12 m | MCA205 |
| P15 | Lung | 0 | NR | <12 m | MCA205 |
| P16 | Lung | 0.46892 | NR | ≥12 m | MCA205 |
| P17 | Lung | 0.24693 | NR | ≥12 m | MCA205 |
| P18 | Lung | 0.72853 | NR | ≥12 m | MCA205 |
| P19 | Lung | 0 | NR | <12 m | MCA205 |
| P20 | Lung | 0 | NR | ≥12 m | MCA205 |
| P21 | Lung | 2.18628 | NR | <12 m | CT26, MC38, 4T1 |
| P22 | Lung | 0 | R | ≥12 m | B16F10, 4T1 |
| P23 | Lung | 0 | NR | <12 m | MCA205 |
| P24 | Lung | 0 | NR | <12 m | MC38, B16F10, 4T1 |
| P25 | Lung | 0 | NR | ≥12 m | MCA205 |
| P26 | Lung | 0 | NR | <12 m | MCA205, 4T1 |
| P27 | Lung | 0 | NR | <12 m | MCA205 |
| P29 | Lung | 7.32764 | R | ≥12 m | MCA205 |
| P30 | Lung | 0 | NR | <12 m | MCA205 |
| P31 | Lung | 13.30026 | NR | <12 m | MCA205 |
| P32 | Lung | 22.53562 | NR | <12 m | MCA205 |

Table illustrating information presented in FIG. 9.

### Akk, the Intestinal Ecosystem and the Tumor Microenvironment

Given known correlations between compositional differences in the gut microbiota and tumor immune landscape (Routy, Le Chatelier, et al. 2018; Gopalakrishnan et al. 2018), that can vary across histological types (Meng et al. 2019), we addressed the interactions between stool Akk detection and tumour histology (squamous versus non-squamous NSCLC). The presence of Akk in stools at diagnosis had no influence on histology (non-squamous vs squamous NSCLC (for multivariate analysis, p=0.556 in Table 5A).

In order to uncover intratumoral transcriptomic differences driven by Akk, we performed tumor RNA sequencing in a subset of patients with available tumor biopsies harvested from Akk⁺ (n=22) and Akk⁻ (n=22) patients upon diagnosis of locally advanced or metastatic NSCLC (Table 3). The supervised analysis of significant gene expression differences between Akk⁺ and Akk⁻ groups within a panel of 395 immune-related genes of the Oncomine Immune Response Research Assay (Hwang et al. 2020) revealed a set of differentially expressed genes associated with response to PD-1 blockade in lung cancer (FIG. 1F) such as CD4⁺ T helper cells with activation (CD4, CD74, Vcam-1, associated with adhesion and trans-migration of T lymphocytes within tumor nests (Nakajima et al. s. d.), granzyme H serine protease Gzmh associated with cytolytic activity) and exhaustion (Ctla4, Fas, Tigit, Havcr2) markers and the IFN fingerprint (Ccr5, Cxcl9, Cxcl10, Tdo2, and IFN-inducible guanylate binding protein 1) (FIG. 1G-H). These results support the possibility that Akk could promote the elicitation or recirculation of Th1 cells into the tumor microenvironment, as previously shown in mouse models (Routy, Le Chatelier, et al. 2018; Vetizou et al. 2015).

Next, we examined compositional taxonomic differences in the gut microbiota in Akk⁺ versus Akk⁻ patients. We found a significant increase of the Shannon diversity index (FIG. 6A upper panel, p<0.0001) in Akk⁺ compared to Akk⁻ patients, as well as differences in the overall microbial community composition between both groups (FIG. 6A lower panel, p=0.0001). Using LEfSe (Linear discriminant analysis Effect Size) analyses to investigate differences in species relative abundance between the 2 groups, we found Ruminococcacae (Ruminococcus bromii, R. bicirculans, R. lactaris), Lachnospiraceae (Eubacterium siraeum, E. eligens) family members and Alistipes spp. (A. inops, A. finegoldii, A. indistinctus, A. shahii) to be enriched in Akk⁺ stools as previously reported, (Hakozaki et al. 2020) while feces from Akk⁻ patients were overabundant in Veillonella parvula, Actinomyces and genus Clostridium (C. innocuum, Hungatella hathewayi) (FIG. 6B) as already described in the lower airway microbiome of patients with NSCLC with poor prognosis (Tsay et al. 2020) as well as patients with kidney cancer resistant to ICI (Lisa Derosa et al. 2020).

Furthermore, within the Akk⁺ group, we found significant differences in the overall microbial composition between patients with OS 12 versus OS<12 months, but this difference was not observed in Akk patients (FIG. 2A, p=0.009 versus p=0.07). LEfSe analysis within the Akk⁺ group exhibiting OS 12 months versus those with OS<12 months unveiled increased relative abundance of Lachnospiraceae family members (Dorea formicigenerans, D. longicatena, Eubacterium rectale, E. hallii, R. intestinalis, Coprococcus comes) in patients with OS 12. Conversely, species belonging to the Gammaproteobacteria (E. coli), Clostridia class (R. lactatiformans) or Bacilli class (such as Lactobacillus gasseri, L. paragasseri, L. oris, L. vaginalis, Streptococcus parasanguinis) and Veillonella parvula were dominant in patients with OS<12 (FIG. 6C).

Taken together, these results indicate that the presence of Akk is associated with important, potentially prognosis-relevant shifts in the intestinal microbiota and tumor microenvironment of NSCLC patients.

### Stratification of Clinical Outcome Based on the Relative Abundance of Akk

We unexpectedly found an over-representation of Akk in patients with OS<12 months within the Akk⁺ group, suggesting that the relative abundance of Akk may influence prognosis more than its absolute presence or absence. We next examined an ordinal rather than a categorical (Akk⁺ versus Akk⁻) variable to analyze the clinical significance of Akk. Indeed, the relative abundance of Akk within the Akk⁺ population ranged from 0.035% up to 66.210%. Using a Kernel density estimation of the relative abundance of Akk positioning patients with OS≥12 or OS<12, we noticed that patients harboring Akk^{high}, at a relative abundance >75^{th} percentile (4.656%), did cluster within the OS<12 months (FIG. 2B-C left panel, p=0.005). To confirm this observation adjusting for the other risk factors, we used a supervised approach to statistically define an optimal cutoff for Akk to discriminate two hypothetical groups of Akk⁺ patients with different prognosis. We utilized a grid search algorithm based on the multivariable Cox model (adjusted for age, sex, ECOG performance status, ATB, histology, PD-L1 and line of treatment), with the Akaike information criterion as a performance index to determine this finest cutoff that corresponded to 4.799 (the 77^{th} percentile of this cohort) (FIG. 2C left panel). Among the whole cohort of 338 patients, we observed that only 9% of patients (23% of Akk⁺ subgroup) fell into the category of “relative overabundance” of Akk>4.799 apostrophed “Akk^{high}” henceforth (FIG. 2C right panel).

Moreover, we found a significant increase in Shannon diversity in Akk^{low} compared to Akk⁻ or Akk^{high} specimens (FIG. 7A, p=0.00003), with the overall microbial community showing clear separation between Akk⁻ versus Akk^{low} as well as between Akk^{low} versus Akk^{high} patients (FIG. 7B-C, p=0.0001 and p=0.0003, respectively). Variable importance plot (VIP) discriminant analysis of taxonomic stool composition revealed that the ecosystem abnormally enriched in Akk (Akk^{high}) was overrepresented by species of the genus Clostridium (Clostridium symbosium, C. innocuum, C. scindens, C. boltae, C. clostridioforme) at the expense of healthy commensals (F. prausntzii, C. comes, E. rectale, D. formicigenerans, D. longicatena, R. torques, FIG. 7D-E).

Kaplan Meier survival curves using the trichotomic stratification according to Akk relative abundance diverged (logrank test p=0.0007, FIG. 2D), with a significantly longer median OS for Akk^{low} patients compared to Akk^{high} (27.2 months versus 7.8 months, Cox adjusted HR:0.38; 95% CI: 0.22-0.65, p=0.0005) and Akk⁻ patients (27.2 months versus 15.5 months, adjusted HR:0.63; 95% CI:0.44-0.91, p=0.0150). The HR for the other risk factors considered in the multivariate Cox regression analysis are presented in FIG. 2E and Table 4. Not surprisingly, ECOG performance status 1 was another independent prognostic factor for this cohort (Desai et al, 2016). The proportionality hazard assumption was questionable only for the ATB (Schoenfeld residuals trend test p=0.016), but no statistically significant interaction with time could be identified. In addition, ATB exposure was associated with shorter OS (in univariable analysis, p=0.009 (Table 4B, Table 7), and multivariate analysis, p=0.088 (Table 7).

We also analyzed the interaction between Akk and PD-L1 in 235 advanced NSCLC patients with an available tumor expression of tumor PD-L1 (Table 5A, FIG. 4). We split the cohort into six groups according to PD-L1 expression and Akk fecal detection. The multivariate Cox regression analysis indicated that Akk dictated NSCLC prognosis, more than PD-L1 did (p=0.012 in 235 patients, FIG. 2F).

Overall, we conclude that considering the trichotomic stratification of patients into Akk⁻, Akk^{low} or Akk^{high} individuals may be a more accurate independent prognostic factor of overall survival than the dichotomic (Akk⁻ versus Akk⁺) division (likelihood ratio test of multivariable Cox models: p=0.0009). The presence of “normal levels” of Akk in the gut (Akk^{low}) may be considered as a surrogate of host intestinal fitness. Akk overruled PD-L1 as a predictive biomarker of response to ICI in NSCLC patients.

TABLE 7
Uni-Multi-variate analyses for OS

| | | | Univariate | | Multivariate | |
|---|---|---|---|---|---|---|
| | | N | HR (CI 95%) | P-value | HR (CI 95%) | P-value |
| A. muciniphila | No | 207 | Reference | | Reference | |
| | Low | 101 | 0.56 (0.39-0.8) | 0.002 | 0.63 (0.44-0.92) | 0.015 |
| | High | 30 | 1.58 (1.00-2.5) | 0.049 | 1.67 (1.03-2.71) | 0.038 |
| Age | (continuous) | 338 | 1.00 (0.98-1.0) | 0.656 | 0.99 (0.98-1.01) | 0.379 |
| Sex | Female | 112 | Reference | | Reference | |
| | Male | 226 | 0.88 (0.65-1.2) | 0.437 | 0.88 (0.63-1.21) | 0.419 |
| ECOG | 0 | 122 | Reference | | Reference | |
| Performance | 1 | 146 | 2.00 (1.41-2.9) | 0.000114 | 1.78 (1.23-2.57) | 0.002 |
| status | >2 | 43 | 2.49 (1.59-3.9) | 7.43e−05 | 2.27 (1.40-3.67) | 0.0005 |
| | Unknown | 27 | 0.87 (0.39-1.9) | 0.7 | 0.84 (0.37-1.89) | 0.676 |
| ATB | No | 269 | Reference | | Reference | |
| | Yes | 69 | 1.6 (1.1-2.3) | 0.009 | 1.36 (0.95-1.94) | 0.088 |
| Histology | Non- | 260 | Reference | | Reference | |
| | Squamous | | | | | |
| | Squamous | 78 | 1.1 (0.81-1.6) | 0.467 | 1.11 (0.78-1.58) | 0.556 |
| PD-L1 | <1% | 64 | Reference | | Reference | |
| | 1-49% | 53 | 1.10 (0.65-1.9) | 0.733 | 1.11 (0.65-1.91) | 0.708 |
| | ≥50% | 118 | 0.99 (0.63-1.5) | 0.957 | 0.90 (0.53-1.54) | 0.713 |
| | Unknown | 102 | 1.40 (0.92-2.1) | 0.117 | 1.22 (0.78-1.89) | 0.382 |
| Immunotherapy | 1 | 98 | Reference | | Reference | |
| Line | 2 | 240 | 1.1 (0.8-1.6) | 0.485 | 0.87 (0.54-1.4) | 0.575 |

ATB, Antibiotics.
P-values were calculated using the Wald test including each covariate separately in a Cox Proportional Hazards Regression Model.
Significant P-value were marked in bold.

### The Impact of ATB Use on Akk Relative Abundance and Survival Outcome

Akk^{high} levels as well as ATB exposure were considered standalone variables associated with shorter OS in NSCLC patients treated with ICI. Given these observations, we first combined the dichotomic classification of patients with respect to Akk (Akk⁻ versus Akk⁺) with their history of prior antibiotic exposure to segregate patients into four groups. The Akk⁺ group without ATB exposure showed the strongest clinical benefit (median OS of 23.0 months) compared to the three other groups (FIG. 8A). The next favorable prognostic group fell into Akk without ATB exposure, with a median OS of 16.0 months. In contrast, exposure to ATB showed the shortest OS (mOS around 9 months, FIG. 8A, p=0.017). Accordingly, ATB tended to reduce the alpha diversity of the Akk⁺ group (FIG. 8B-C). Moreover, ATB exposure enriched the Akk⁺ group in Gammaproteobacteria (E. coli), Clostridia class (Clostridium bolteae, Ruthenibacterium lactatiformens), and H2S producing bacteria (Bilophila wadsworthia) (not show), as already described (Lisa Derosa et al. 2020) at the expense of health-associated bacteria (C. aerofaciens, D. longicatena, D. formicigenerans, Eubacterium sp. CAG 38)(Routy, Gopalakrishnan, et al. 2018; Benevides et al. 2017) also over-represented in the Akk⁺ group who did not take ATB (not shown).

In an attempt to establish an association between ATB use and the relative overabundance of Akk, we compared the percentages of Akk^{high} stools in NSCLC patients with ATB use versus those that were ATB-free (FIG. 2G) and performed a Kaplan Meier OS curve using the trichotomic classification (Akk⁻, Akk^{low} versus Akk^{high}) in two subgroups according to ATB exposure before the first administration of ICI (FIG. 2H). We found a drastic increase of the proportion of Akk^{high} patients among ATB users versus non users (40% versus 19%, p=0.023, FIG. 2G). We confirmed that the overabundance of stool Akk at diagnosis in patients exposed to ATB was associated with lower overall survival despite ICI therapy (FIG. 8, FIG. 2H bottom panel). In ATB-free subjects, Akk^{high} patients exhibited a reduced benefit to ICI compared with Akk^{low} individuals but not worse than the Akk^{neg} subgroup (FIG. 2H top panel).

Altogether, these results demonstrate that ATB exposure is a negative predictor of survival to ICI, associated with overabundance of Akk (Akk^{high}) and the relative dominance of Clostridium spp (C. bolteae, Lachnoclostridium).

### Stratification of Outcome on Other Components of Akk-Associated Ecosystem

We used various statistical methods such as Linear discriminant analysis effect size (FIG. 6), Volcano plot from ANOVA model (FIG. 3A), and MaAsLin multivariate statistical framework (Table 8) to compare taxonomic compositional variations between Akk⁺ versus Akk⁻ stools in order to identify 16 bacterial species, among which 14 were positively and 2 were negatively associated with Akk fecal prevalence independently of other confounding factors such as age, gender, BMI, lines of therapy (FIG. 3A, Table 8). Next, we performed Cox logistic regression multivariate analyses to determine the impact of each bacterium on patient clinical outcome using a trichotomic distribution of their respective relative abundances (according to a cut-off defined above). Apart from Akk, very few bacteria (except Eubacterium hallii, Bifidobacterium adolescentis, Parasutterella excrementihominis, Intestinimonas butyriciproducens, or C. innocuum already described to mediate immunomodulatory functions (Elkrief et al, 2019; Seo et al, 2015; Atarashi et al, 2011) were also associated with prolonged (FIG. 3E, FIG. 3D) or reduced survival following ICI therapy (FIG. 3F, Table 8) with a “linear dose” response in contrast to what was observed for Akk. There was an added value to consider the coordinated presence of both, Akk as well as E. hallii or B. adolescentis, to accurately predict ORR and/or mOS in this cohort of 338 NSCLC patients (FIG. 3C,E). Hence, these findings suggest that Akk and its collateral commensalism may participate in the ICI-mediated clinical benefit of patients.

TABLE 8
A. muciniphila-associated intestinal commensalism potentially relevant to predict overall survival.

| | | | | | PKM | |
|---|---|---|---|---|---|---|
| MaAsLin2 | | | | | trichotomic | PKM Binary |
| adjP | LogFC | CI. UP | CI. L | Taxa | distribution | with Akk |
| 5.578e−08 | 2.1311 | 2.4577 | 1.80457 | Akkermansia_{—}muciniphila | | |
| 0.0012 | 1.8706 | 2.2980 | 1.44327 | Intestinimonas_{—}butyriciproducens | 0.19 | Not done |
| 0.0012 | 1.6276 | 2.0011 | 1.2542 | Ruminococcaceae_bacterium_D5 | 0.1217 | Not done |
| 0.0019 | 1.4563 | 1.8048 | 1.1078 | Eubacterium_{—}hallii | 0.0180 | 0.0265 |
| 0.0237 | 1.2512 | 1.6163 | 0.8860 | Roseburia_{—}hominis | Not done | Not done |
| 0.0237 | 1.5531 | 2.0039 | 1.1023 | Ruminococcaceae_bacterium_D16 | Not done | Not done |
| 0.0295 | 1.2471 | 1.6226 | 0.8716 | Coprobacter_{—}fastidiosus | 0.0344 | 0.1290 |
| 0.0311 | 0.8399 | 1.0967 | 0.5830 | Bacteroides_{—}plebeius | Not done | Not done |
| 0.0343 | 1.0605 | 1.3916 | 0.7294 | Anaerostipes_{—}hadrus | 0.0059 | 0.06 |
| 0.0356 | 0.9916 | 1.3102 | 0.6731 | Bacteroides_{—}ovatus | Not done | Not done |
| 0.0356 | 1.2895 | 1.7081 | 0.8710 | Alistipes_{—}indistinctus | 0.23 | Not done |
| 0.0356 | 0.8481 | 1.1229 | 0.5734 | Eubacterium_{—}eligens | Not done | Not done |
| 0.0356 | −1.3844 | −0.9363 | −1.8324 | Clostridium_{—}innocuum | <0.0001 | 0.1190 |
| 0.0379 | 1.1870 | 1.5777 | 0.79632 | Parasutterella_{—}excrementihominis | 0.0063 | Not done |
| 0.0432 | −1.1987 | −0.7960 | −1.6014 | Dielma_{—}fastidiosa | Not done | Not done |
| 0.0466 | 0.9117 | 1.2226 | 0.6008 | Bifidobacterium_{—}adolescentis | 0.0181 | 0.0391 |
| 0.0487 | 1.1409 | 1.5344 | 0.7474 | Harryflintia_{—}acetispora | Not done | Not done |

adjP, Adjusted P-value using MaAslin2 multivariate analysis; LogFC, Log Fold Change; CI, Confidence Interval; UP, upper; L, Lower; PKM, P-value Kaplan-Meier.
Table illustrating information presented in FIG. 3.

### Akk Rescued Response to PD-1 Blockade by Shifting the Microbiome

Trying to establish a link between Akk and/or its collateral ecosystem and patient clinical outcome, we turned to our microbiota humanized avatar tumor bearing mouse models described above. As depicted and commented above (FIG. 5C-E), mouse resistance to anti-PD-1 antibodies was associated with FMT bereft of Akk. Next, we analyzed the compensatory effects brought up by oral supplementation with an immunogenic strain of Akkermansia called Akkermansia p2261 (Routy, Le Chatelier, et al. 2018) in both groups of animals, receiving Akk⁺ versus Akk⁻ FMT. Mice reared in specific pathogen-free conditions (SPF), indicated as FMT—in FIG. 9A-1B) and inoculated with the same tumor cell line were used as controls of “murine eubiosis”. Indeed, we could restore responsiveness to PD-1 blockade, only in the setting of Akk⁻ FMT (FIG. 9B-C). Next, the objective was to analyze whether oral supplementation with Akkermansia p2261 would change the host microbiome of the recipient mice and whether the remodeling of the host ecosystem would correlate with preclinical benefit to exogenous Akkermansia p2261. For this purpose, we concatenated all tumor models syngeneic of BALB/c (CT26, 4T1) and C57BL/6 (B16F10, MCA-205 MC38) mice that were first transferred with stools from 29 individual NSCLC patients (Tablex S3) and then treated with anti-PD-1 antibodies (FIG. 9A) and collected recipient feces pre- and post-oral feeding with Akkermansia p2261. The non-supervised hierarchical clustering of the ratios between the tumor growth kinetics with anti-PD-1 antibodies with or without exogenous Akkermansia p2261 normalized on the effect of PD-1 blockade in eubiotic conditions (SPF versus FMT) revealed that exogenous Akkermansia p2261 was effective in about 50% cases (FIG. 9D, FIG. 9E left panel). We detected taxonomic differences in the recipient microbiota in responders (R) versus non responders (NR) to exogenous Akkermansia p2261 (FIG. 9E right panel). R mice harbored a relative overrepresentation of metagenomics species annotated in FIG. 3A in humans harboring Akk⁺ stools namely Intestinimonas butyriciproducens, Parasuterrella excrementihominis compared with NR mice, that in contrast, tended to be enriched in Bacteroides spp (FIG. 9E right panel).

Altogether, avatar mice transferred with Akk⁻ human fecal material exhibited a phenotype of tumor resistance to PD-1 blockade but were rescued by Akkermansia p2261 when Akkermansia p2261 could shift the microbiome towards the favorable Akk associated collateral ecosystem.

### Discussion

Here, we report the results of a prospective, multicentric study based on the profiling of the gut microbiota of patients with advanced NSCLC treated with PD-1 blockade. The relative abundance of Akk was associated with clinical benefit, defined by an increase in ORR and survival, taking into accounting the main microbiota-relevant confounding factors (age, gender, BMI, lines of therapy). The prognostic significance of this gut bacterium was validated by multivariate analyses and interaction studies indicating that Akk is markedly associated to the prognosis of advanced NSCLC treated with ICI, independently from age, gender, ECOG PS, ATB use and PD-L1. The intestinal residence of Akk was a proxy of richness of the gut ecosystem, as shown by the association of Akk at a relative abundance within the 77^{th} percentile (Akk^{low}<4.799%), with stool alpha diversity (Shannon diversity index). These results expand on previous observations that have been made in smaller cohorts of patients with NSCLC (Routy, Le Chatelier, et al. 2018; Hakozaki et al. 2020) and provide evidence that gut microbiome diversity and composition, specifically the relative abundance of Akk, offer relevant information to predict survival of patients with NSCLC amenable to ICI.

Our study is the largest metagenomics prospective analysis that attempted to validate Akk as a new prognostic factor for ICI. Our study meet the pre-specified criterium of statistical significance which was set at 10% ORR increase between Akk⁻ and Akk⁺ patients when considering all (mostly 2L) 338 NSCLC patients (from 18.2% to 28.2% in ORR, FIG. 1A). Moreover, we also observed a >10% ORR increase between Akk⁻ and Akk⁺ (from 19% to 41% ORR, FIG. 1B) with a mOS advantage in 1L NSCLC patients (FIG. 1E). Moreover, our study linked the gut microbiota composition to the tumor microenvironmental landscape, highlighting increased transcription of gene products of the adaptive immunity and the IFN fingerprint. Finally, we validated in mice that Akk⁻ stools conferred resistance to PD-1 blockade.

In addition to prospectively validating the hypothesis in a larger and homogeneous cohort, we report that Akk was associated not only with increased alpha diversity but also with a distinct bacterial community associated with a health or immunogenic status represented by Ruminococcacae (Faecalibacterium prausnitzii, R. lactaris) and Lachnospiraceae (Dorea formicigenerans & D. longicatena, Eubacterium rectale & E. hallii, Roseburia faecis & R. intestinalis) family members as well as Bifidobacterium adolescentis, Intestinimonas butyricyproducens and others. These findings reconcile the results across several works, geographical distributions and sequencing technologies since Faecalibacterium, Ruminococcus and Bifidobacterium were previously reported to be enriched in North-American, Japanese and South Korean patients with melanoma and NSCLC cancers and have favorable outcome (Routy, Gopalakrishnan, et al. 2018; Gopalakrishnan et al. 2018; Hakozaki et al. 2020), (Lee et al. 2021). Moreover, even in avatar gut humanized mouse models, responders to exogenous Akkermansia p2261 shifted their microbiome towards an over-representation of some of the above mentioned species belonging to the Akk⁺ ecosystem.

Confirming the clinical significance of bacterial diversity and commensals associated with responses, our results validate the growing body of evidence linking ATB use and poor clinical outcome (Elkrief et al. 2019). In addition to depleting favorable genera associated with survival (such as Ruminococcus) (Hakozaki et al. 2020), ATB use tends to deviate the gut microbiome composition towards harmful bacteria previously associated with proinflammatory or immunoregulatory pathways (such as E. coli, and Clostridium bolteae) (Seo et al. 2015), supporting previous findings in renal cell carcinomas amenable to ICI (Lisa Derosa et al. 2020). Surprisingly, in addition to acting as an independent negative prognostic factor, ATB promoted the overabundance of Akk (Akk^{high}) above the 77^{th} percentile level associated with poor prognosis. Indeed, ATB use doubled the proportion of individuals presenting a stool Akk^{high} phenotype. This phenotypic trait of overabundance of Akk>4.799 was associated with a dominance of the Clostridium species (C. bolteae, C. innocuum, C. asparagiforme, C. scindens, C. symbosium) belonging to clusters IV and XIVa of the genus Clostridium, known to maintain IL-10 producing Treg in colonic lamina propria (Atarashi et al. 2011).

Aside from ATB use, overabundance of Akk>4.799 (Akk^{high}) was associated with a shorter overall survival than “normal” relative abundance of Akk<4.799, possibly reflecting an underlying pathophysiological disorder of the intestinal barrier in these advanced cancer patients. High relative proportions or subdominance of A. muciniphila in the ecosystem has been associated with pathophysiological failures (such as anorexia nervosa (Ruusunen et al. 2019), GVHD (Shono et al. 2016), Aging (van der Lugt et al. 2019), dysmetabolism (Depommier et al. 2019b), HIV infection (Ouyang et al. 2020), pathobionts (Huck et al. 2020) or liver injury (Wu et al. 2017)). Hence, in the context of gut injury by, or conducive to, ATB use, Akk might constitute a biomarker of ongoing but imperfect intestinal repair. Of note, we failed to observe a similar trichotomic distribution correlating with opposite clinical outcome investigating other bacteria (FIG. 3).

Hence, we conclude that Akk relative abundance could represent a reliable biomarker of favorable or dismal prognosis for patients receiving immunotherapy with PD-1 blockade. It may be of utmost importance to risk-stratified I-O patients based on shot-gun metagenomics (rather than by 16S rRNA) sequencing to precisely quantify the relative abundance of Akk in addition to ATB use, and PD-L1 expression in prospective trials including NSCLC patients and designed to discover optimal biomarkers.

Therapeutic strategies modulating the microbiome such as FMT or commensals are currently being evaluated to boost ICI responses or circumvent primary resistance to ICI, though without patient stratification based on their degree of dysbiosis (Baruch et al. 2020). Here, we provide preclinical data suggesting that Akk could therapeutically bypass the resistance to anti-PD-1 blockade conferred by FMT bereft of endogenous Akk (FIG. 9 and (Routy, Gopalakrishnan, et al. 2018; Routy, Le Chatelier, et al. 2018)). The oral supplementation with exogenous Akkermansia (Akkermansia p2261) was particularly effective when it shifted the recipient microbiome towards a healthy status (FIG. 9D-E). A recent preclinical study indicates that the anticancer effects of recombinant interleukin-2 could be improved by Akk in a Toll-like receptor-2 dependent fashion (Shi et al. 2020). In patients, food supplementation with Akk is safe and reduces insulinoresistance and dyslipidemia in the context of pre-diabetes (Depommier et al. 2019a). Altogether, we surmise that therapeutic supplementation with a lyophilized encapsulated Akkermansia would benefit the subgroups of patients not exposed to ATB, and devoid of endogenous Akk. In contrast, complex polymicrobial consortia or fecal microbial transplantation may be best suited for patients with prior ATB exposure.

Hence, our study provides a strong rationale for the development of diagnostic tools assessing gut dysbiosis for routine oncological management, as well as framework for the design of microbiota-centered interventions to circumvent primary resistance to ICI in patients with NSCLC.

### Example 2: Intestinal Akkermansia SGB9228 Predicts Clinical Response to PD1 Blockade in Advanced Non-Small Cell Lung Cancer Patients and Boosts the Efficacy of PD-1 Blockade in Preclinical Models

### Background:

Recent works published by several groups have suggested the presence of several clades (Becken et al., mBio 2021) or species-level (Karcher et al., Genome Biol. 2021; Guo et al., BMC Genomics 2017) of Akkermansia. Indeed, and as suggested by Karcher et al., a total of five Akkermansia candidate species including Akkermansia muciniphila exist in the human, mouse, and non-human primate gut microbiomes, four of them remaining under-investigated and uncharacterized (Akkermansia SGB9223, SGB9224, SGB9227, and SGB9228). Of note, these strains displayed high similarity by 16S rRNA gene sequences, with 16S rRNA gene sequences of strains in different candidate species never diverging by more than 2%. Akkermansia candidate species differed strongly in their prevalence across hosts. A. muciniphila is by far the most prevalent candidate species across all hosts, being detected in 34% of adult humans and reaching a maximum prevalence of 54% in laboratory-held mice. The other candidate species were detected at lower prevalence (<25%) across all hosts.

### Relevance of Akkermansia SGB9228 Species in Driving the Clinical Efficacy of PD1-Blocklade in Cancer Patients:

We were able to extract genomic markers and profile 4 out of these 5 SGBs in our cohort and therefore better recapitulate Akkermansia genomic diversity as follows. As outlined in Example 1 (Table 2, showing the prevalence of various subspecies of Akkermansia in the cohort of 338 NSCLC cancer patients), SGB9226 (Akkermansia muciniphila MucT) is the most prevalent species in our cohort (>80% of Akkermansia positive subset).

Of note, the prevalence reported for the clade SGB9226 (proxy for A. muciniphila) and SGB9228 does not differ from the one reported by Karcher et al. in the general human population, demonstrating that the presence of a particular clade is not involved in the pathological process of NSCLC (Karcher et al., Genome Biol. 2021). We finally compared the predictive value of the presence of SGB9226 versus SGB9228 clade to drive the clinical efficacy of ICI in NSCLC cancer patients. Considering that the trichotomic stratification of patients into A. muciniphila⁻, A. muciniphila^{low} or A. muciniphila^{high} individuals may be a more accurate independent prognostic factor of overall survival than the dichotomic (A. muciniphila⁻ versus A. muciniphila⁺) division, the same analysis was conducted using SGB9228. As shown in FIG. 10, Akkermansia SGB9228 behave the same way as Akkermansia muciniphila, although the number of patients SGB9228+ included in this analysis was significantly lower. Altogether, the presence of “normal levels” of A. muciniphila in the gut (A. muciniphila^{low}) or Akkermansia SGB9228^{low} may be considered as a surrogate of host intestinal fitness (FIG. 10).

Akkermansia p2261 is a Strain of Akkermansia Belonging to Akkermansia SGB9228:

Accordingly, a deep characterization of Akkermansia p2261 has been performed to elucidate the phylogroup and respective phenotypic traits of Akkermansia p2261. Whole-genome phylogeny of the metagenome-assembled genomes was performed, and it was determined that Akkermansia p2261 belongs to the species Akkermansia SGB9228, which is distinct from Akkermansia muciniphila.

Akkermansia muciniphila type strain genome provided by the American Type Culture Collection (ATCC) was compared to Akkermansia p2261 genome. The completeness of assembled isolates was evaluated using CheckM's lineage_wf function (Parks et al., Genome Res. 2015). The assembled contigs were then processed and Parsnp was used as the core genome aligner to align the core genome of multiple microbial genomes. The average nucleotide identity (ANIm) between isolates was assessed using MUMmer (Kurtz et al., Genome Biol. 2004). As demonstrated in Table 9, although these strains displayed high similarity by 16S rRNA gene sequences, genome-wide average estimated nucleotide identities between Akkermansia p2261 (genome G1284) and Akkermansia muciniphila ATCC below 90%, demonstrating that Akkermansia strain p2261 and Akkermansia muciniphila are genetically distinct.

TABLE 9
Average identity via MUMer. Average Identity indicates the nucleotide
identity percentage between Akkermansia muciniphipla ATCC BAA 835 type
strain genome and the G1284 reference (Akkermansia p2261). A higher
average identity indicates greater similarity between genomes.

| Reference Genome | Query Genome | AverageIdentity |
|---|---|---|
| G1284_PE1MN1_spades | Akkermansia_muciniphila_ATCC_BAA_835 | 88.7 |

Akkermansia SGB9228 strain p2261 and 4531 safely boost the efficacy of PD-1 blockade in preclinical models: A deep evaluation of the capacity of strains of Akkermansia (including strain 3284, 5126, 5801, 4531 and 2261) to safety boost the preclinical efficacy of immune checkpoint blockers was performed. The capacity of Akkermansia strains to safely ameliorate the efficacy of PD1 blockade in MCA205 tumor-bearing mice transplanted with the fecal material of a NR NSCLC cancer patient was investigated (FIG. 11). Akkermansia strains 2261 and 4531 were potent in boosting the antitumoral efficacy of PD-1 blockade.

We also monitored the secretion of IL-12 cytokine by bone marrow derived dendritic cells (BMDCs) that were previously pulsed with different strains of Akkermansia (including strain 3284, 5126, 5801, 4531 and p2261) (FIG. 12). Akkermansia strains 2261 and 4531 were potent in boosting the secretion of IL-12 by BMDCs.

### Design of Specific Primers Recognizing Akkermansia Species:

Primers have been designed for the specific identification of Akkermansia muciniphila (SGB9226) and Akkermansia SGB9228 species (FIG. 13). The primers were designed with Primer-BLAST (Ye et al., BMC Bioinformatics, 2012) and the specificity was checked using representative genomes deposited on RefSeq.

The relative abundances of Akkermansia muciniphila and/or Akkermansia SGB9228 can be measured by quantitative PCR using the following primers:

TABLE 10
primers specific for Akkermansia muciniphila and/or Akkermansia SGB9228

| Name | Sequence | SEQ ID No. | Specificity |
|---|---|---|---|
| AkkermansiaSGB9226/9228_F | TTCGCAACGGACGAAGTGTA | 1 | A. muciniphila + |
| AkkermansiaSGB9226/9228_R | TCCGTATACGTGTCCCCGAT | 2 | A. SGB9228 |
| AkkermansiaSGB9226_F | CGGAGCCGAAAATACCCAGA | 3 | Akkermansia |
| AkkermansiaSGB9226_R | TCACCTTTGGCAAGTTCATCCT | 4 | muciniphila |
| AkkermansiaSGB9228_F | GGCTGAAAACACCCAGATGC | 5 | Akkermansia |
| AkkermansiaSGB9228_R | TGGCGAGTTCGTCCTTCAAC | 6 | SGB9228 |
| Akker_rpob_F | GCAACAGGGTCTTGGTGATT | 7 | Akkermansia |
| Akker_rpob_R | GCAGCTCATTGACCAGTTGA | 8 | muciniphila |
| p2261 forward | TGCGTATGGTGGGGATATCC | 9 | Akkermansia |
| p2261 reverse | CCTTCAGTCCGTTCTCCACT | 10 | strain P2261 |

### REFERENCES

Atarashi, Koji, Takeshi Tanoue, Tatsuichiro Shima, Akemi Imaoka, Tomomi Kuwahara, Yoshika Momose, Genhong Cheng, et al. 2011. «Induction of Colonic Regulatory T Cells by Indigenous Clostridium Species». Science (New York, N. Y.) 331 (6015): 337-41. https://doi.org/10.1126/science.1198469.
Bárcena, Clea, Rafael Valdés-Mas, Pablo Mayoral, Cecilia Garabaya, Sylvère Durand, Francisco Rodríguez, Maria Teresa Fernández-García, et al. 2019. «Healthspan and Lifespan Extension by Fecal Microbiota Transplantation into Progeroid Mice». Nature Medicine 25 (8): 1234-42. https://doi.org/10.1038/s41591-019-0504-5.
Baruch, Erez N., Ilan Youngster, Guy Ben-Betzalel, Rona Ortenberg, Adi Lahat, Lior Katz, Katerina Adler, et al. 2020. «Fecal Microbiota Transplant Promotes Response in Immunotherapy-Refractory Melanoma Patients». Science, décembre. https://doi.org/10.1126/science.abb5920.
Becken, B. et al. Genotypic and Phenotypic Diversity among Human Isolates of Akkermansia muciniphila. mBio 12, e00478-21 (2021).
Beghini, Francesco, Lauren J. McIver, Aitor Blanco-Míguez, Leonard Dubois, Francesco Asnicar, Sagun Maharjan, Ana Mailyan, et al. 2020. «Integrating Taxonomic,
Functional, and Strain-Level Profiling of Diverse Microbial Communities with BioBakery 3». BioRxiv, novembre, 2020.11.19.388223. https://doi.org/10.1101/2020.11.19.388223.
Benevides, Leandro, Sriti Burman, Rebeca Martin, Véronique Robert, Muriel Thomas, Sylvie Miquel, Florian Chain, et al. 2017. «New Insights into the Diversity of the Genus Faecalibacterium». Frontiers in Microbiology 8: 1790. https://doi.org/10.3389/fmicb.2017.01790.
Blacher, Eran, Stavros Bashiardes, Hagit Shapiro, Daphna Rothschild, Uria Mor, Mally Dori-Bachash, Christian Kleimeyer, et al. 2019. «Potential Roles of Gut Microbiome and Metabolites in Modulating ALS in Mice». Nature 572 (7770): 474-80. https://doi.org/10.1038/s41586-019-1443-5.
Borghaei, Hossein, Luis Paz-Ares, Leora Horn, David R. Spigel, Martin Steins, Neal E. Ready, Laura Q. Chow, et al. 2015. «Nivolumab versus Docetaxel in Advanced Nonsquamous Non-Small-Cell Lung Cancer». New England Journal of Medicine 373 (17): 1627-39. https://doi.org/10.1056/NEJMoa1507643.
Brahmer, Julie, Karen L. Reckamp, Paul Baas, Lucio Crinò, Wilfried E. E. Eberhardt, Elena Poddubskaya, Scott Antonia, et al. 2015. «Nivolumab versus Docetaxel in Advanced Squamous-Cell Non-Small-Cell Lung Cancer». New England Journal of Medicine 373 (2): 123-35. https://doi.org/10.1056/NEJMoa1504627.
Bray, Nicolas L., Harold Pimentel, Páll Melsted, et Lior Pachter. 2016. «Near-Optimal Probabilistic RNA-Seq Quantification». Nature Biotechnology 34 (5): 525-27. https://doi.org/10.1038/nbt.3519.
Cani, Patrice D., et Willem M. de Vos. 2017. «Next-Generation Beneficial Microbes: The Case of Akkermansia Muciniphila». Frontiers in Microbiology 8. https://doi.org/10.3389/fmicb.2017.01765.
Chen, Daniel S., et Ira Mellman. 2017. «Elements of Cancer Immunity and the Cancer-Immune Set Point». Nature 541 (7637): 321-30. https://doi.org/10.1038/nature21349.
Daisley, Brendan A., Ryan M. Chanyi, Kamilah Abdur-Rashid, Kait F. Al, Shaeley Gibbons, John A. Chmiel, Hannah Wilcox, et al. 2020. «Abiraterone Acetate Preferentially Enriches for the Gut Commensal Akkermansia Muciniphila in Castrate-Resistant Prostate Cancer Patients». Nature Communications 11 (1): 4822. https://doi.org/10.1038/s41467-020-18649-5.
Depommier, Clara, Amandine Everard, Céline Druart, Hubert Plovier, Matthias Van Hul, Sara Vieira-Silva, Gwen Falony, et al. 2019a. «Supplementation with Akkermansia Muciniphila in Overweight and Obese Human Volunteers: A Proof-of-Concept Exploratory Study». Nature Medicine 25 (7): 1096-1103. https://doi.org/10.1038/s41591-019-0495-2.—2019b. «Supplementation with Akkermansia Muciniphila in Overweight and Obese Human Volunteers: A Proof-of-Concept Exploratory Study». Nature Medicine 25 (7): 1096-1103. https://doi.org/10.1038/s41591-019-0495-2.
Derosa, L., M. D. Hellmann, M. Spaziano, D. Halpenny, M. Fidelle, H. Rizvi, N. Long, et al. 2018. «Negative Association of Antibiotics on Clinical Activity of Immune Checkpoint Inhibitors in Patients with Advanced Renal Cell and Non-Small-Cell Lung Cancer». Annals of Oncology: Official Journal of the European Society for Medical Oncology 29 (6): 1437-44. https://doi.org/i0.1093/annonc/mdy103.
Derosa, Lisa, Bertrand Routy, Antoine Desilets, Romain Daillère, Safae Terrisse, Guido Kroemer, et Laurence Zitvogel. 2021. «Microbiota-Centered Interventions: The Next Breakthrough in Immuno-Oncology?» Cancer Discovery 11 (10): 2396-2412. https://doi.org/10.1158/2159-8290.CD-21-0236.
Derosa, Lisa, Bertrand Routy, Marine Fidelle, Valerio Iebba, Laurie Alla, Edoardo Pasolli, Nicola Segata, et al. 2020. «Gut Bacteria Composition Drives Primary Resistance to Cancer Immunotherapy in Renal Cell Carcinoma Patients». European Urology 78 (2): 195-206. https://doi.org/10.1016/j.eururo.2020.04.044.
Derosa, Lisa, et Laurence Zitvogel. 2020. «Antibiotics Impair Immunotherapy for Urothelial Cancer». Nature Reviews. Urology 17 (11): 605-6. https://doi.org/i0.1038/s41585-020-0373-1.
Desai, Mahesh S., Anna M. Seekatz, Nicole M. Koropatkin, Nobuhiko Kamada, Christina A. Hickey, Mathis Wolter, Nicholas A. Pudlo, et al. 2016. «A Dietary Fiber-Deprived Gut Microbiota Degrades the Colonic Mucus Barrier and Enhances Pathogen Susceptibility». Cell 167 (5): 1339-1353.e21. https://doi.org/10.1016/j.cell.2016.10.043.
Eisenhauer, E. A., P. Therasse, J. Bogaerts, L. H. Schwartz, D. Sargent, R. Ford, J. Dancey, et al. 2009. «New Response Evaluation Criteria in Solid Tumours: Revised RECIST Guideline (Version 1.1)». European Journal of Cancer (Oxford, England: 1990) 45 (2): 228-47. https://doi.org/10.1016/j.ejca.2008.10.026.
Elkrief, A., L. Derosa, G. Kroemer, L. Zitvogel, et B. Routy. 2019. (The Negative Impact of Antibiotics on Outcomes in Cancer Patients Treated with Immunotherapy: A New Independent Prognostic Factor?» Annals of Oncology: Official Journal of the European Society for Medical Oncology 30 (10): 1572-79. https://doi.org/10.1093/annonc/mdz206.
Ferrara, Roberto, Laura Mezquita, Matthieu Texier, Jihene Lahmar, Clarisse Audigier-Valette, Laurent Tessonnier, Julien Mazieres, et al. 2018. «Hyperprogressive Disease in Patients With Advanced Non-Small Cell Lung Cancer Treated With PD-1/PD-L1 Inhibitors or With Single-Agent Chemotherapy». JAMA Oncology 4 (11): 1543-52. https://doi.org/10.1001/jamaoncol.2018.3676.
Fumet, Jean-David, Corentin Richard, Fanny Ledys, Quentin Klopfenstein, Philippe Joubert, Bertrand Routy, Caroline Truntzer, et al. 2018. «Prognostic and Predictive Role of CD8 and PD-L1 Determination in Lung Tumor Tissue of Patients under Anti-PD-1 Therapy». British Journal of Cancer 119 (8): 950-60. https://doi.org/10.1038/s41416-018-0220-9.
Gadgeel, Shirish, Delvys Rodríguez-Abreu, Giovanna Speranza, Emilio Esteban, Enriqueta Felip, Manuel Dómine, Rina Hui, et al. 2020. «Updated Analysis From KEYNOTE-189: Pembrolizumab or Placebo Plus Pemetrexed and Platinum for Previously Untreated Metastatic Nonsquamous Non-Small-Cell Lung Cancer». Journal of Clinical Oncology: Official Journal of the American Society of Clinical Oncology 38 (14): 1505-17. https://doi.org/10.1200/JCO.19.03136.
Gandhi, L., D. Rodríguez-Abreu, S. Gadgeel, E. Esteban, E. Felip, F. De Angelis, M. Domine, et al. 2018. «Pembrolizumab plus Chemotherapy in Metastatic Non-Small-Cell Lung Cancer». New England Journal of Medicine 378 (22): 2078-92. https://doi.org/10.1056/NEJMoa1801005.
Gopalakrishnan, V., C. N. Spencer, L. Nezi, A. Reuben, M. C. Andrews, T. V. Karpinets, P. A. Prieto, et al. 2018. «Gut Microbiome Modulates Response to Anti-PD-1 Immunotherapy in Melanoma Patients». Science (New York, N.Y.) 359 (6371): 97-103. https://doi.org/10.1126/science.aan4236.
Guo, X. et al. Genome sequencing of 39 Akkermansia muciniphila isolates reveals its population structure, genomic and functional diversity, and global distribution in mammalian gut microbiotas. BMC Genomics 18, 800 (2017).
Hakozaki, Taiki, Corentin Richard, Arielle Elkrief, Yukio Hosomi, Myriam Benlaïfaoui, Iris Mimpen, Safae Terrisse, et al. 2020. «The Gut Microbiome Associates with Immune Checkpoint Inhibition Outcomes in Patients with Advanced Non-Small Cell Lung Cancer». Cancer Immunology Research 8 (10): 1243-50. https://doi.org/10.1158/2326-6066.CIR-20-0196.
Herbst, Roy S., Paul Baas, Dong-Wan Kim, Enriqueta Felip, José L. Pérez-Gracia, Ji-Youn Han, Julian Molina, et al. 2016. «Pembrolizumab versus Docetaxel for Previously Treated, PD-L1-Positive, Advanced Non-Small-Cell Lung Cancer (KEYNOTE-010): A Randomised Controlled Trial». The Lancet 387 (10027): 1540-50. https://doi.org/10.1016/S0140-6736(15)01281-7.
Huck, Olivier, Hannah Mulhall, George Rubin, Zev Kizelnik, Radha Iyer, John D. Perpich, Nasreen Haque, Patrice D. Cani, Willem M. de Vos, et Salomon Amar. 2020. ((Akkermansia Muciniphila Reduces Porphyromonas Gingivalis-Induced Inflammation and Periodontal Bone Destruction». Journal of Clinical Periodontology 47 (2): 202-12. https://doi.org/10.1111/jcpe.13214.
Hwang, Sohyun, Ah-Young Kwon, Ju-Yeon Jeong, Sewha Kim, Haeyoun Kang, Joonsuk Park, Joo-Hang Kim, Ok Jin Han, Sun Min Lim, et Hee Jung An. 2020. «Immune Gene Signatures for Predicting Durable Clinical Benefit of Anti-PD-1 Immunotherapy in Patients with Non-Small Cell Lung Cancer». Scientific Reports 10 (1): 643. https://doi.org/10.1038/s41598-019-57218-9.
Jin, Yueping, Hui Dong, Liliang Xia, Yi Yang, Yongqiang Zhu, Yan Shen, Huajun Zheng, Chengcheng Yao, Ying Wang, et Shun Lu. 2019. «The Diversity of Gut Microbiome Is Associated With Favorable Responses to Anti-Programmed Death 1 Immunotherapy in Chinese Patients With NSCLC». Journal of Thoracic Oncology: Official Publication of the International Association for the Study of Lung Cancer 14 (8): 1378-89. https://doi.org/10.1016/j.jtho.2019.04.007.
Karcher, Nicolai, Eleonora Nigro, Michal Punc̆ochár̆, Aitor Blanco-Míguez, Matteo Ciciani, Paolo Manghi, Moreno Zolfo, et al. 2021. «Genomic diversity and ecology of human-associated Akkermansia species in the gut microbiome revealed by extensive metagenomic assembly». Genome Biology 22 (1): 209. https://doi.org/10.1186/s13059-021-02427-7.
Koyama, Shohei, Esra A. Akbay, Yvonne Y. Li, Grit S. Herter-Sprie, Kevin A. Buczkowski, William G. Richards, Leena Gandhi, et al. 2016. «Adaptive Resistance to Therapeutic PD-1 Blockade Is Associated with Upregulation of Alternative Immune Checkpoints». Nature Communications 7 (février): 10501. https://doi.org/10.1038/ncomms10501.
Kurtz, S. et al. Versatile and open software for comparing large genomes. Genome Biol 5, R12 (2004).
Le Chatelier, Emmanuelle, Trine Nielsen, Junjie Qin, Edi Prifti, Falk Hildebrand, Gwen Falony, Mathieu Almeida, et al. 2013. «Richness of Human Gut Microbiome Correlates with Metabolic Markers». Nature 500 (7464): 541-46. https://doi.org/10.1038/nature12506.
Lee, Se-Hoon, Sung-Yup Cho, Youngmin Yoon, Changho Park, Jinyoung Sohn, Jin-Ju Jeong, Bu-Nam Jeon, et al. 2021. «Bifidobacterium Bifidum Strains Synergize with Immune Checkpoint Inhibitors to Reduce Tumour Burden in Mice». Nature Microbiology 6 (3): 277-88. https://doi.org/10.1038/s41564-020-00831-6.
Li, Junhua, Huijue Jia, Xianghang Cai, Huanzi Zhong, Qiang Feng, Shinichi Sunagawa, Manimozhiyan Arumugam, et al. 2014. «An Integrated Catalog of Reference Genes in the Human Gut Microbiome». Nature Biotechnology 32 (8): 834-41. https://doi.org/10.1038/nbt.2942.
Lin, Huang, et Shyamal Das Peddada. 2020. «Analysis of Compositions of Microbiomes with Bias Correction». Nature Communications 11 (1): 3514. https://doi.org/i0.1038/s41467-020-17041-7.
Lugt, Benthe van der, Adriaan A. van Beek, Steven Aalvink, Ben Meijer, Bruno Sovran, Wilbert P. Vermeij, Renata M. C. Brandt, et al. 2019. «Akkermansia muciniphila ameliorates the age-related decline in colonic mucus thickness and attenuates immune activation in accelerated aging Ercc1-/Δ7 mice». Immunity & Ageing 16 (1): 6. https://doi.org/10.1186/s12979-019-0145-z.
Mallick, Rishav, Judith A. Hubbard, Eric O. Lindsey, Kyle E. Bradley, James D. P. Moore, Aktarul Ahsan, A. K. M. Khorshed Alam, et Emma M. Hill. 2020. «Subduction Initiation and the Rise of the Shillong Plateau». Earth and Planetary Science Letters 543 (août): 116351. https://doi.org/10.1016/j.epsl.2020.116351.
Matson, Vyara, Jessica Fessler, Riyue Bao, Tara Chongsuwat, Yuanyuan Zha, Maria-Luisa Alegre, Jason J. Luke, et Thomas F. Gajewski. 2018. «The Commensal Microbiome Is Associated with Anti-PD-1 Efficacy in Metastatic Melanoma Patients». Science (New York, N. Y.) 359 (6371): 104-8. https://doi. org/10.1126/science.aao3290.
Meng, Xiangjiao, Yongsheng Gao, Lian Yang, Haiyan Jing, Feifei Teng, Zhaoqin Huang, et Ligang Xing. 2019. «Immune Microenvironment Differences Between Squamous and Non-Squamous Non-Small-Cell Lung Cancer and Their Influence on the Prognosis». Clinical Lung Cancer 20 (1): 48-58. https://doi.org/10.1016/j.cllc.2018.09.012.
Nakajima, Kosei, Yoshinori Ino, Rie Yamazaki-Itoh, Chie Naito, Mari Shimasaki, Mami Takahashi, Minoru Esaki, et al. s. d. «IAP inhibitor, Embelin increases VCAM-1 levels on the endothelium, producing lymphocytic infiltration and antitumor immunity». Oncoimmunology 9 (1). Consulté le 12 décembre 2020. https://doi.org/10.1080/2162402X.2020.1838812.
Nielsen, H. Bjørn, Mathieu Almeida, Agnieszka Sierakowska Juncker, Simon Rasmussen, Junhua Li, Shinichi Sunagawa, Damian R. Plichta, et al. 2014. «Identification and Assembly of Genomes and Genetic Elements in Complex Metagenomic Samples without Using Reference Genomes». Nature Biotechnology 32 (8): 822-28. https://doi.org/i0.1038/nbt.2939.
Ouyang, Jing, John Lin, Stephane Isnard, Brandon Fombuena, Xiaorong Peng, André Marette, Bertrand Routy, Meriem Messaoudene, Yaokai Chen, et Jean-Pierre Routy. 2020. «The Bacterium Akkermansia Muciniphila: A Sentinel for Gut Permeability and Its Relevance to HIV-Related Inflammation». Frontiers in Immunology 11: 645. https://doi.org/10.3389/fimmu.2020.00645.
Parks, D. H., Imelfort, M., Skennerton, C. T., Hugenholtz, P. & Tyson, G. W. CheckM: assessing the quality of microbial genomes recovered from isolates, single cells, and metagenomes. Genome Res 25, 1043-1055 (2015).
Paz-Ares, Luis, Alexander Luft, David Vicente, Ali Tafreshi, Mahmut Gümüş, Julien Mazières, Barbara Hermes, et al. 2018. «Pembrolizumab plus Chemotherapy for Squamous Non-Small-Cell Lung Cancer». New England Journal of Medicine 379 (21): 2040-51. https://doi.org/10.1056/NEJMoa1810865.
Reck, Martin, Delvys Rodríguez-Abreu, Andrew G. Robinson, Rina Hui, Tibor Csöszi, Andrea Fülöp, Maya Gottfried, et al. 2016. «Pembrolizumab versus Chemotherapy for PD-L1-Positive Non-Small-Cell Lung Cancer». New England Journal of Medicine 375 (19): 1823-33. https://doi.org/10.1056/NEJMoa1606774.
Riaz, Nadeem, Jonathan J. Havel, Vladimir Makarov, Alexis Desrichard, Walter J. Urba, Jennifer S. Sims, F. Stephen Hodi, et al. 2017. «Tumor and Microenvironment Evolution during Immunotherapy with Nivolumab». Cell 171 (4): 934-949.e16. https://doi.org/10.1016/j.cell.2017.09.028.
Rizvi, Naiyer A., Matthew D. Hellmann, Alexandra Snyder, Pia Kvistborg, Vladimir Makarov, Jonathan J. Havel, William Lee, et al. 2015. «Cancer Immunology. Mutational Landscape Determines Sensitivity to PD-1 Blockade in Non-Small Cell Lung Cancer». Science (New York, N.Y.) 348 (6230): 124-28. https://doi.org/10.1126/science.aaa1348.
Romano, Stefano, George M. Savva, Janis R. Bedarf, Ian G. Charles, Falk Hildebrand, et Arjan Narbad. 2021. «Meta-Analysis of the Parkinson's Disease Gut Microbiome Suggests Alterations Linked to Intestinal Inflammation». Npj Parkinson's Disease 7 (1): 1-13. https://doi.org/10.1038/s41531-021-00156-z.
Routy, Bertrand, Vancheswaran Gopalakrishnan, Romain Daillère, Laurence Zitvogel, Jennifer A. Wargo, et Guido Kroemer. 2018. «The Gut Microbiota Influences Anticancer Immunosurveillance and General Health». Nature Reviews. Clinical Oncology 15 (6): 382-96. https://doi.org/i0.1038/s41571-018-0006-2.
Routy, Bertrand, Emmanuelle Le Chatelier, Lisa Derosa, Connie P. M. Duong, Maryam Tidjani Alou, Romain Daillère, Aurélie Fluckiger, et al. 2018. «Gut Microbiome Influences Efficacy of PD-1-Based Immunotherapy against Epithelial Tumors». Science (New York, N. Y.) 359 (6371): 91-97. https://doi. org/10.1126/science.aan3706.
Ruusunen, Anu, Tetyana Rocks, Felice Jacka, et Amy Loughman. 2019. «The gut microbiome in anorexia nervosa: relevance for nutritional rehabilitation». Psychopharmacology 236 (5): 1545-58. https://doi.org/10.1007/s00213-018-5159-2.
Santoro, Aurelia, Rita Ostan, Marco Candela, Elena Biagi, Patrizia Brigidi, Miriam Capri, et Claudio Franceschi. 2018. «Gut Microbiota Changes in the Extreme Decades of Human Life: A Focus on Centenarians». Cellular and Molecular Life Sciences: CMLS 75 (1): 129-48. https://doi.org/10.1007/s00018-017-2674-y.
Segata, Nicola, Jacques Izard, Levi Waldron, Dirk Gevers, Larisa Miropolsky, Wendy S. Garrett, et Curtis Huttenhower. 2011. «Metagenomic Biomarker Discovery and Explanation». Genome Biology 12 (6): R60. https://doi.org/10.1186/gb-2011-12-6-r60.
Seo, Sang Woo, Donghyuk Kim, Richard Szubin, et Bernhard O. Palsson. 2015. «Genome-Wide Reconstruction of OxyR and SoxRS Transcriptional Regulatory Networks under Oxidative Stress in Escherichia Coli K-12 MG1655». Cell Reports 12 (8): 1289-99. https://doi.org/10.1016/j.celrep.2015.07.043.
Shi, Linlin, Jianyong Sheng, Guozhong Chen, Peng Zhu, Changping Shi, Bei Li, Chaiwoo Park, et al. 2020. «Combining IL-2-Based Immunotherapy with Commensal Probiotics Produces Enhanced Antitumor Immune Response and Tumor Clearance». Journal for Immunotherapy of Cancer 8 (2). https://doi.org/10.1136/jitc-2020-000973.
Shono, Yusuke, Melissa D. Docampo, Jonathan U. Peled, Suelen M. Perobelli, Enrico Velardi, Jennifer J. Tsai, Ann E. Slingerland, et al. 2016. «Increased GVHD-Related Mortality with Broad-Spectrum Antibiotic Use after Allogeneic Hematopoietic Stem Cell Transplantation in Human Patients and Mice». Science Translational Medicine 8 (339): 339ra71. https://doi.org/10.1126/scitranslmed.aaf2311.
Smyth, Mark J., Shin Foong Ngiow, Antoni Ribas, et Michele W. L. Teng. 2016. «Combination Cancer Immunotherapies Tailored to the Tumour Microenvironment». Nature Reviews. Clinical Oncology 13 (3): 143-58. https://doi.org/10.1038/nrclinonc.2015.209.
Spranger, Stefani, Riyue Bao, et Thomas F. Gajewski. 2015. «Melanoma-Intrinsic β-Catenin Signalling Prevents Anti-Tumour Immunity». Nature 523 (7559): 231-35. https://doi.org/i0.1038/nature14404.
Tsay, Jun-Chieh J., Benjamin G. Wu, Imran Sulaiman, Katherine Gershner, Rosemary Schluger, Yonghua Li, Ting-An Yie, et al. 2020. «Lower Airway Dysbiosis Affects Lung Cancer Progression». Cancer Discovery, novembre. https://doi.org/10.1158/2159-8290.CD-20-0263.
Vétizou, Marie, Jonathan M. Pitt, Romain Daillère, Patricia Lepage, Nadine Waldschmitt, Caroline Flament, Sylvie Rusakiewicz, et al. 2015. «Anticancer Immunotherapy by CTLA-4 Blockade Relies on the Gut Microbiota». Science (New York, N.Y.) 350 (6264): 1079-84. https://doi.org/10.1126/science.aad1329.
Wu, Wenrui, Longxian Lv, Ding Shi, Jianzhong Ye, Daiqiong Fang, Feifei Guo, Yating Li, Xingkang He, et Lanjuan Li. 2017. «Protective Effect of Akkermansia muciniphila against Immune-Mediated Liver Injury in a Mouse Model». Frontiers in Microbiology 8: 1804. https://doi.org/10.3389/fmicb.2017.01804.
Ye, J. et al. Primer-BLAST: a tool to design target-specific primers for polymerase chain reaction. BMC Bioinformatics 13, 134 (2012).
Young, Arabella, Shin Foong Ngiow, Deborah S. Barkauskas, Erin Sult, Carl Hay, Stephen J. Blake, Qihui Huang, et al. 2016. «Co-Inhibition of CD73 and A2AR Adenosine Signaling Improves Anti-Tumor Immune Responses». Cancer Cell 30 (3): 391-403. https://doi.org/10.1016/j.ccell.2016.06.025.
Zhou, Qi, Yanfeng Zhang, Xiaoxia Wang, Ruiyue Yang, Xiaoquan Zhu, Ying Zhang, Chen Chen, Huiping Yuan, Ze Yang, et Liang Sun. 2020. «Gut bacteria Akkermansia is associated with reduced risk of obesity: evidence from the American Gut Project». Nutrition & Metabolism 17 (1): 90. https://doi.org/10.1186/s12986-020-00516-1.

1. An in vitro theranostic method of determining if a cancer patient is likely to be a good responder to an immune checkpoint inhibitor (ICI)-based therapy, comprising measuring, in a sample from said patient, the relative abundance of bacteria of the Akkermansia genus, wherein the presence of bacteria of the Akkermansia genus below a predetermined threshold is indicative that the patient is likely to be a good responder to the ICI-based therapy.

2. The method of claim 1, wherein the presence of bacteria of the Akkermansia genus above the predetermined threshold is indicative of dismal prognosis despite ICI-based therapy.

3. The method of claim 1, for determining if a cancer patient needs a bacterial compensation before administration of an ICI-based therapy, comprising measuring, in a sample from said patient, the relative abundance of bacteria of the Akkermansia genus, wherein:
(i) If no Akkermansia is present in the sample, the patient needs a bacterial compensation with at least bacteria of the Akkermansia genus before ICI administration,
(ii) if bacteria of the Akkermansia genus are present in the sample below a predetermined threshold, the patient does not need any bacterial compensation before ICI administration; and
(iii) if bacteria of the Akkermansia genus are present in the sample above a predetermined threshold, especially if this overrepresentation is consecutive to antibiotics exposure and/or associated with an overrespresentation of species belonging to the Gammaproteobacteria class and/or to the Desulfovibrionaceae family, the patient needs a bacterial compensation with a polymicrobial consortium and/or through fecal microbial transplant (FMT) from a healthy individual or from a cancer patient who successfully responded to the ICI-based therapy.

4. A method for determining if an individual has an intestinal microbiota dysbiosis, comprising measuring, in a sample from said individual, the relative abundance of bacteria of the Akkermansia genus, wherein the presence of bacteria of the Akkermansia genus below a predetermined threshold is indicative that there is no intestinal microbiota dysbiosis.

5. The method of claim 1, wherein the relative abundance of Akkermansia muciniphila and/or Akkermansia SGB9228 is measured and compared to at least one predetermined threshold.

6. The method of claim 1, wherein the predetermined threshold corresponds to a relative abundance between 1 and 10%.

7. The method of claim 1, wherein the predetermined threshold corresponds to a relative abundance between 3 and 6.5%.

8. The method of claim 1, wherein the ICI-based therapy is an anti-PD1/PD-L1/PD-L2 Ab-based therapy.

9. The method of claim 1, wherein the patient suffers from non small cell lung cancer (NSCLC) or kidney cancer.

10. The method of claim 1, wherein the patient suffers from non-squamous NSCLC.

11. The method of claim 1, wherein the ICI-based therapy is administered as first-line therapy or second-line therapy.

12. A method according to claim 1, wherein the sample from said patient or individual is a feces sample, a sample from the colon or ileal luminal content of said patient or individual or a mucosal biopsy from said patient or individual.

13. A composition for use in combination with an ICI-based therapy comprising:
a) a fecal microbial composition for use in the treatment of a cancer patient having an overrepresentation of Akkermansia in his/her intestinal microbiota, or
b) a bacterial composition comprising bacteria of the Akkermansia genus, for use in the treatment of a cancer patient having no Akkermansia in his/her intestinal microbiota.

14. (canceled)

15. The fecal microbial composition or the bacterial composition of claim 13, wherein said bacteria of the Akkermansia genus are Akkermansia SGB9228.

16. The fecal microbial composition or the bacterial composition of claim 13, wherein the patient suffers from non small cell lung cancer (NSCLC) or kidney cancer.

17. The fecal microbial composition or the bacterial composition of claim 13, wherein the patient suffers from non-squamous NSCLC.


SEQUENCE LISTING (10 sequences)

SEQ ID NO: 1 - DNA, 20 bp, Artificial Sequence
  AkkermansiaSGB9226/9228_F primer
ttcgcaacggacgaagtgta

SEQ ID NO: 2 - DNA, 20 bp, Artificial Sequence
  AkkermansiaSGB9226/9228_R primer
tccgtatacgtgtccccgat

SEQ ID NO: 3 - DNA, 20 bp, Artificial Sequence
  AkkermansiaSGB9226_F primer
cggagccgaaaatacccaga

SEQ ID NO: 4 - DNA, 22 bp, Artificial Sequence
  AkkermansiaSGB9226_R primer
tcacctttggcaagttcatcct

SEQ ID NO: 5 - DNA, 20 bp, Artificial Sequence
  AkkermansiaSGB9228_F primer
ggctgaaaacacccagatgc

SEQ ID NO: 6 - DNA, 20 bp, Artificial Sequence
  AkkermansiaSGB9228_R primer
tggcgagttcgtccttcaac

SEQ ID NO: 7 - DNA, 20 bp, Artificial Sequence
  Akker_rpob_F primer
gcaacagggtcttggtgatt

SEQ ID NO: 8 - DNA, 20 bp, Artificial Sequence
  Akker_rpob_R primer
gcagctcattgaccagttga

SEQ ID NO: 9 - DNA, 20 bp, Artificial Sequence
  p2261 forward primer
tgcgtatggtggggatatcc

SEQ ID NO: 10 - DNA, 20 bp, Artificial Sequence
  p2261 reverse primer
ccttcagtccgttctccact